(19) United States
(12) Patent Application Publication
(10) Pub. No.: US 2025/0002607 A1
(43) Pub. Date: Jan. 2, 2025

(54) BISPECIFIC ANTIBODIES

(71) Applicant: Novo Nordisk A/S, Bagsvaerd (DK)

(72) Inventors: Prafull S. Gandhi, Ballerup (DK); Jens Breinholt, Dyssegaard (DK); Henrik Østergaard, Ølsted (DK)

(21) Appl. No.: 18/827,611

(22) Filed: Sep. 6, 2024

Related U.S. Application Data
(63) Continuation of application No. 17/699,460, filed on Mar. 21, 2022, now Pat. No. 12,122,847.
Application No. 17/699,460 is a division of application No. 17/501,548, filed on Oct. 14, 2021, now Pat. No. 11,325,983.
Application No. 17/501,548 is a continuation of application No. PCT/EP2020/060579, filed on Apr. 15, 2020.

(30) Foreign Application Priority Data
Apr. 17, 2019 (EP) 19169704.4
Dec. 5, 2019 (EP) 19213867.5

(51) Int. Cl. C07K 16/36; A61K 39/00; A61P 7/04; C07K 16/28

(52) U.S. Cl. CPC C07K 16/36; A61P 7/04; C07K 16/2803; A61K 39/00; A61K 2039/505; C07K 2317/31; C07K 2317/56; C07K 2317/565

(57) ABSTRACT

The invention relates to a bispecific antibody comprising a first antigen-binding site capable of binding Factor VII(a) and a second antigen-binding site capable of binding TLT-1, pharmaceutical formulations comprising such bispecific antibodies and uses thereof.

### CROSS-REFERENCE

This application is a continuation of U.S. application Ser. No. 17/699,460, filed Mar. 21, 2022, which is a division of U.S. application Ser. No. 17/501,548, filed Oct. 14, 2021, now issued as U.S. Pat. No. 11,325,983 on May 10, 2022, which is a continuation of International Application No. PCT/EP2020/060579, filed Apr. 15, 2020, which claims the benefit of and priority to European Application No. 19169704.4, filed Apr. 17, 2019, and European Application No. 19213867.5, filed on Dec. 5, 2019, the contents of each of which are hereby incorporated by reference in their entirety for all purposes.

### SEQUENCE LISTING

The contents of the electronic sequence listing (HMT-001USC1_SL.xml; Size: 1,173,504 bytes; and Date of Creation: Jul. 22, 2024) is herein incorporated by reference in its entirety.

### TECHNICAL FIELD OF THE INVENTION

The present invention pertains to bispecific antibodies exhibiting improved pharmaceutical properties, compositions comprising such antibodies and uses of such antibodies and compositions, such as pharmaceutical and therapeutic uses.

### BACKGROUND OF THE INVENTION

The process of blood coagulation involves several proteins that act together following vascular injury to generate a blood clot that prevents severe loss of body fluids and/or pathogenic invasion. The cascade of events leading to the formation of a blood clot can be initiated through two pathways known as the intrinsic (contact) and extrinsic (tissue factor) pathways. Each pathway consists of a series of zymogen activation steps where a newly activated enzyme catalyzes the activation of the next zymogen in the series until prothrombin is converted to thrombin. Thrombin coverts fibrinogen to fibrin meshwork and activates platelets to form a platelet plug, together resulting in the formation of a stable blood clot. Initiation of the extrinsic clotting pathway is mediated by formation of the complex between membrane bound tissue factor (TF), which is exposed as a result of injury to a vessel wall, and low levels of circulating factor VIIa (FVIIa). The FVIIa:TF complex initiates the coagulation cascade by activating small quantities of coagulation factor IX (FIX) and factor X (FX). During the initial phase, the small concentration of FXa produces minute amounts of thrombin that can activate factor XI and cofactors VIII and V. During the propagation phase, the procoagulant complexes are assembled and they significantly enhance the generation of FXa and thrombin by the tenase (FIXa, FVIIIa, Ca²⁺, phospholipids) and prothrombinase (FXa, FVa, Ca²⁺, phospholipids) complexes respectively.

In people with haemophilia A and B (HA and HB, respectively) various steps of the coagulation cascade are rendered dysfunctional due to the absence or insufficient presence of functional FVIII and FIX, respectively. This leads to an impaired and insufficient blood coagulation and potentially life-threatening bleeding or damage to internal organs, such as the joints.

Recombinant FVIIa (rFVIIa) has been widely used as a bypassing agent for on-demand (OD) treatment of bleeds in people with haemophilia (A and B) with inhibitors (HwI). rFVIIa has a short systemic half-life of 2-3 hrs when administered intravenously (IV) and a low bioavailability when administered subcutaneously (SC). The short systemic half-life of rFVIIa is believed to be due to involvement of several mechanisms including inhibition by plasma inhibitors antithrombin III (AT) (Agersø H et al. (2010) J. Thromb. Haemost. 9:333-8.) and alpha-2-macroglobulin (α2M) as well as renal clearance. The short systemic half-life and low SC bioavailability of rFVIIa makes it challenging to utilize rFVIIa for prophylactic treatment. Additionally, low intrinsic activity of rFVIIa requires administration of higher rFVIIa doses.

Consequently, there is a need for improved compounds that can support prophylactic treatment with less frequent dosing and SC administration.

Roche recently launched a bispecific antibody, emicizumab, that is indicated for routine prophylaxis dosed once-weekly through SC administration for people with HA and HA with inhibitors (HAwI). Nonetheless, development of safe, efficacious molecules with alternative mechanism of action remains an area of key interest in order to improve and supplement the standard of care for people with Haemophilia.

### SUMMARY OF INVENTION

The present invention relates to bispecific antibodies exhibiting improved pharmaceutical properties, particularly it relates to bispecific antibodies that could be used in the treatment of subjects with congenital and/or acquired coagulopathies, for example in the treatment of people with hemophilia A or B with or without inhibitors. Further, the present invention particularly pertains to bispecific antibodies that are capable of binding coagulation Factor VII (FVII(a)) and TREM-like Transcript 1 (TLT-1).

In one aspect, the bispecific antibodies of the invention comprise (i) a first antigen binding site that binds FVII(a), and (ii) a second antigen binding site that binds TLT-1.

In an aspect of the invention, the bispecific antibody prolongs the active circulatory half-life of endogenous FVIIa without loss in endogenous FVIIa activity and stimulate endogenous FVIIa activity by localizing it selectively to activated platelets.

In an embodiment of the invention, the bispecific antibody comprises a Fc region. The Fc region mediates recycling of the bispecific antibody prolonging its half-life in circulation.

In an embodiment of the invention, the first antigen-binding site competes for binding to FVII(a) in a competitive ELISA assay with any one of the anti-FVII(a) antibodies comprising a light chain variable domain (VL) and a heavy chain variable domain (VH) as indicated below:
mAb0522 (VL: SEQ ID NO 846 and VH: SEQ ID NO: 850),
Fab0883 (VL: SEQ ID NO 814 and VH: SEQ ID NO: 818),
mAb0005 (VL: SEQ ID NO 750 and VH: SEQ ID NO: 754),
mAb0004 (VL: SEQ ID NO 14 and VH: SEQ ID NO: 18),
mAb0013 (VL: SEQ ID NO 46 and VH: SEQ ID NO: 50),
mAb0018 (VL: SEQ ID NO 62 and VH: SEQ ID NO: 66),
mAb0544 (VL: SEQ ID NO 694 and VH: SEQ ID NO: 698),
mAb0552 (VL: SEQ ID NO 702 and VH: SEQ ID NO: 706),
mAb0001 (VL: SEQ ID NO 710 and VH: SEQ ID NO: 714),
mAb0007 (VL: SEQ ID NO 718 and VH: SEQ ID NO: 722),
mAb0578 (VL: SEQ ID NO 726 and VH: SEQ ID NO: 730),
mAb0701 (VL: SEQ ID NO 734 and VH: SEQ ID NO: 738), and
mAb0587 (VL: SEQ ID NO 742 and VH: SEQ ID NO: 746).

In a further embodiment of the invention, the first antigen-binding site of the bispecific antibody is capable of binding an epitope comprising amino acid residues H115, T130, V131, and R392 of FVII(a) (SEQ ID NO: 1).

In an aspect of the invention, the bispecific antibodies are formulated in pharmaceutical formulations comprising a bispecific antibody of the invention and a pharmaceutically acceptable carrier.

In an aspect of the invention, the bispecific antibodies are parenterally administered, such as intravenously, intramuscularly or subcutaneously. In an aspect of the invention the bispecific antibodies allow for the prophylactic treatment of subjects with a congenital and/or acquired coagulopathy, such as haemophilia A, haemophilia B, haemophilia A with inhibitors or haemophilia B with inhibitors. Hence, the bispecific antibodies are designed to provide haemostatic coverage against bleeds. In an aspect of the invention the bispecific antibodies are designed such as to be suitable for once weekly, once monthly or less frequent administration.

Medical treatment with the bispecific antibodies of the invention may offer a number of advantages, such as longer duration between injections, more convenient administration and potentially improved haemostatic protection between injections. Hence, the bispecific antibodies described herein may have a substantial impact upon the quality of life of individuals with haemophilia A or B, with or without inhibitors.

### BRIEF DESCRIPTION OF SEQUENCE LISTING

Table 1 is an overview of antibodies and their corresponding SEQ ID NOs for corresponding VL and VL domain sequences. The type of heavy chain constant domains is as defined in Table 2a (“M” denotes a murine IgG1 constant domain).
Table 2a is an overview of different formats used for recombinant expression of bivalent antibodies, monovalent (OA) antibodies and bispecific antibodies. Listed are SEQ ID NOs corresponding to first heavy chain (HC-1) and second heavy chain (HC-2, or for OA antibodies the truncated heavy chain (trHC)). The light chain constant domain was in all cases human kappa corresponding to SEQ ID NO: 12.
Table 2b is an overview of murine anti-FVII(a) antibodies of the current invention. Correspondence between clone name, abbreviation for fully murine (hybridoma-derived except for mAb0765, which was recombinantly expressed) antibodies and corresponding murine/human chimeric variants (murine variable domains, human IgG4 S228P constant domains).
Table 2c is an overview of antibodies in the murine and humanized 11F2 lineage
Table 2d is an overview of antibodies of the anti-TLT-1 mAb0012 lineage

TABLE 1

| | | VL | VH | Constant |
|---|---|---|---|---|
| Antibody | Target | SEQ ID NO | SEQ ID NO | domain |
| mAb0004 | FVII(a) | 14 | 18 | M |
| mAb0048 | FVII(a) | 22 | 26 | A |
| Fab0076 | FVII(a) | 30 | 34 | G |
| mAb0077(OA) | FVII(a) | 38 | 42 | E |
| mAb0013 | FVII(a) | 46 | 50 | M |
| mAb0099 | FVII(a) | 54 | 58 | F |
| mAb0018 | FVII(a) | 62 | 66 | M |
| mAb0108 | FVII(a) | 70 | 74 | A |
| mAb0109 | FVII(a) | 78 | 82 | A |
| mAb0110 | FVII(a) | 86 | 90 | A |
| mAb0111 | FVII(a) | 94 | 98 | A |
| mAb0112 | FVII(a) | 102 | 106 | A |
| mAb0113 | FVII(a) | 110 | 114 | A |
| mAb0114 | FVII(a) | 118 | 122 | A |
| mAb0115 | FVII(a) | 126 | 130 | A |
| mAb0116 | FVII(a) | 134 | 138 | A |
| mAb0117 | FVII(a) | 142 | 146 | A |
| mAb0118 | FVII(a) | 150 | 154 | A |
| mAb0119 | FVII(a) | 158 | 162 | A |
| mAb0120 | FVII(a) | 166 | 170 | A |
| mAb0121 | FVII(a) | 174 | 178 | A |
| mAb0122 | FVII(a) | 182 | 186 | A |
| mAb0123 | FVII(a) | 190 | 194 | A |
| mAb0124 | FVII(a) | 198 | 202 | A |
| mAb0125 | FVII(a) | 206 | 210 | A |
| mAb0126 | FVII(a) | 214 | 218 | A |
| mAb0127 | FVII(a) | 222 | 226 | A |
| mAb0128 | FVII(a) | 230 | 234 | A |
| mAb0129 | FVII(a) | 238 | 242 | A |
| mAb0130 | FVII(a) | 246 | 250 | A |
| mAb0137(OA) | FVII(a) | 254 | 258 | E |
| mAb0138(OA) | FVII(a) | 262 | 266 | E |
| mAb0139(OA) | FVII(a) | 270 | 274 | E |
| mAb0140(OA) | FVII(a) | 278 | 282 | E |
| mAb0141(OA) | FVII(a) | 286 | 290 | E |
| mAb0142(OA) | FVII(a) | 294 | 298 | E |
| mAb0143(OA) | FVII(a) | 302 | 306 | E |
| mAb0153 | FVII(a) | 310 | 314 | A |
| mAb0154 | FVII(a) | 318 | 322 | A |
| mAb0155 | FVII(a) | 326 | 330 | A |
| mAb0156 | FVII(a) | 334 | 338 | A |
| mAb0157 | FVII(a) | 342 | 346 | A |
| mAb0158 | FVII(a) | 350 | 354 | A |
| mAb0159 | FVII(a) | 358 | 362 | A |
| mAb0160 | FVII(a) | 366 | 370 | A |
| mAb0161 | FVII(a) | 374 | 378 | A |
| mAb0162 | FVII(a) | 382 | 386 | A |
| mAb0163 | FVII(a) | 390 | 394 | A |
| mAb0164 | FVII(a) | 398 | 402 | A |
| mAb0165 | FVII(a) | 406 | 410 | A |
| mAb0166 | FVII(a) | 414 | 418 | A |
| mAb0167 | FVII(a) | 422 | 426 | A |
| mAb0168 | FVII(a) | 430 | 434 | A |
| mAb0169 | FVII(a) | 438 | 442 | A |
| mAb0170 | FVII(a) | 446 | 450 | A |
| mAb0171 | FVII(a) | 454 | 458 | A |
| mAb0172 | FVII(a) | 462 | 466 | A |
| mAb0173 | FVII(a) | 470 | 474 | A |
| mAb0174 | FVII(a) | 478 | 482 | A |
| mAb0175 | FVII(a) | 486 | 490 | A |
| mAb0176 | FVII(a) | 494 | 498 | A |
| mAb0177 | FVII(a) | 502 | 506 | A |
| mAb0178 | FVII(a) | 510 | 514 | A |
| mAb0203 | FVII(a) | 518 | 522 | A |
| mAb0228 | FVII(a) | 526 | 530 | A |
| mAb0253 | FVII(a) | 534 | 538 | A |
| mAb0278 | FVII(a) | 542 | 546 | A |
| mAb0303 | FVII(a) | 550 | 554 | A |
| mAb0328 | FVII(a) | 558 | 562 | A |
| mAb0353 | FVII(a) | 566 | 570 | A |
| mAb0378 | FVII(a) | 574 | 578 | A |
| mAb0403 | FVII(a) | 582 | 586 | A |
| mAb0428 | FVII(a) | 590 | 594 | A |
| mAb0453 | FVII(a) | 598 | 602 | A |
| mAb0478 | FVII(a) | 606 | 610 | A |
| mAb0503 | FVII(a) | 614 | 618 | A |
| mAb0528 | FVII(a) | 622 | 626 | A |
| mAb0553 | FVII(a) | 630 | 634 | A |
| mAb0578 | FVII(a) | 638 | 642 | A |
| mAb0603 | FVII(a) | 646 | 650 | A |
| mAb0705(OA) | FVII(a) | 654 | 658 | F |
| mAb0706(OA) | FVII(a) | 662 | 666 | F |
| mAb0707(OA) | FVII(a) | 670 | 674 | F |
| mAb0709(OA) | FVII(a) | 678 | 682 | F |
| mAb0710(OA) | FVII(a) | 686 | 690 | F |
| mAb0544 | FVII(a) | 694 | 698 | M |
| mAb0552 | FVII(a) | 702 | 706 | M |
| mAb0001 | FVII(a) | 710 | 714 | M |
| mAb0007 | FVII(a) | 718 | 722 | M |
| mAb0578 | FVII(a) | 726 | 730 | M |
| mAb0701 | FVII(a) | 734 | 738 | M |
| mAb0587 | FVII(a) | 742 | 746 | M |
| mAb0005 | FVII(a) | 750 | 754 | M |
| mAb0842(OA) | FVII(a) | 758 | 762 | F |
| mAb0864 | FVII(a) | 766 | 770 | B |
| mAb0865 | FVII(a) | 774 | 778 | B |
| mAb0872 | FVII(a) | 782 | 786 | B |
| mAb0873 | FVII(a) | 790 | 794 | B |
| mAb0874 | FVII(a) | 798 | 802 | B |
| mAb0875 | FVII(a) | 806 | 810 | B |
| Fab0883 | FVII(a) | 814 | 818 | G |
| mAb7250 | FVII(a) | 822 | 826 | M |
| mAb0350 | FVII(a) | 830 | 834 | D |
| mAb0351 | FVII(a) | 838 | 842 | C |
| mAb0522 | FVII(a) | 846 | 850 | K |
| mAb0524 | TLT-1 | 854 | 858 | J |
| mAb0012 | TLT-1 | 862 | 866 | A |
| mAb0023 | TLT-1 | 870 | 874 | A |
| mAb0051 | TLT-1 | 878 | 882 | A |
| mAb0061 | TLT-1 | 886 | 890 | A |
| mAb0062 | TLT-1 | 894 | 898 | A |
| mAb0082 | TLT-1 | 902 | 906 | A |
| mAb1038 | TLT-1 | 910 | 914 | A |
| mAb1047 | TLT-1 | 918 | 922 | A |
| mAb1049 | TLT-1 | 926 | 930 | A |
| mAb1076 | TLT-1 | 934 | 938 | A |

TABLE 2a

| | | | HC-2 |
|---|---|---|---|
| | | HC-1 | (or trHC) |
| Constant | | SEQ | SEQ |
| domain type | Description | ID NO: | ID NO: |
| A | Bivalent antibody | 4 | 4 |
| B | Bivalent antibody (Duobody) | 5 | 5 |
| C | Bivalent antibody (YTE) | 6 | 6 |
| D | Bivalent antibody | 7 | 7 |
| | (Duobody and YTE) | | |
| E | OA antibody (Duobody) | 4 | 8 |
| F | OA antibody (Knob-in-hole) | 9 | 10 |
| G | Fab fragment | 11 | NA |
| H | Bispecific antibody (Duobody) | 5 | 4 |
| I | Bispecific antibody | 7 | 6 |
| | (Duobody and YTE) | | |
| J | Bivalent antibody (delta-Lys) | 942 | 942 |
| K | Bivalent antibody | 943 | 943 |
| | (Duobody, delta-Lys) | | |
| L | Bispecific antibody | 943 | 942 |
| | (Duobody, delta-Lys) | | |

TABLE 2b

| Clone | Murine | Chimera |
|---|---|---|
| 11F2 | mAb0005/mAb0765 | mAb0048 |
| 11F34 | mAb0007 | mAb0760 |
| 11F9 | mAb0544 | mAb0756 |
| 11F18 | mAb0552 | mAb0757 |
| 8F19 | mAb0018 | mAb0100 |
| 11F19 | mAb0004 | mAb0047 |
| 11F26 | mAb0001 | mAb0759 |
| 12F19 | mAb0578 | mAb0762 |
| 12F39 | mAb0587 | mAb0764 |
| 12F25 | mab0013 | mAb0096 |
| 17F227 | mAb0701 | mAb0763 |

TABLE 2c

| Antibody | Variable domain | Constant domain |
|---|---|---|
| mAb0005 | Murine VL/VH | M |
| mAb0048 | Murine VL/VH | A |
| Fab0076 | Murine VL/VH | G |
| mAb0077(OA) | Murine VL/VH | E |
| mAb0705(OA) | Humanized VL/VH | F |
| Fab0883 | Humanized VL/VH | G |
| mAb0865 | Humanized VL/VH | B |
| mAb0522 | Humanized VL/VH | K |

TABLE 2d

| Antibody | Variable domain | Constant domain |
|---|---|---|
| mAb0012 | Murine VL/VH | M |
| mAb0082 | Murine VL C41A/VH T61A | A |
| mAb1076 | Humanized VL/VH | A |
| mAb0524 | Humanized VL/VH | J |

In the present invention recombinantly produced antibodies were all expressed in a human IgG4 background, and all contained the standard hinge-stabilizing substitution S228P (EU numbering). In some variants the C-terminal lysine of the heavy chain (K447, which is rapidly cleaved off in vivo; see Cai et al. Biotechnol. Bioeng. 2011 vol. 108, pp 404-412) was omitted (referred to as delta-lys). Additional substitutions were according to Table 2a introduced in the heavy chain constant domain to secure desired chain-pairing in bispecific and monovalent antibodies (Duobody mutations F405L R409K (Labrijn et al. PNAS 2013, vol. 110, pp. 5145-5150) and knob-in-hole mutations (see Carter et al. J. Imm. Methods 2001, vol. 248, pp. 7-15) T366W (knob) and T366S L368A Y407V (hole)), or to prolong the in vivo half-life (YTE mutations, M252Y S254T T256E (Dall'Acqua et al. J. Biol. Chem. 2006, vol. 18, pp. 23514-23524)). All recombinantly produced antibodies of the present invention has the human kappa light chain constant domain (SEQ ID NO: 12).

### DETAILED DESCRIPTION OF THE INVENTION

The present invention relates to the design and use of antibody compositions exhibiting improved pharmaceutical properties. Particularly it pertains to bispecific antibodies that are capable of binding coagulation FVII(a) and TLT-1.

### Antibodies

The term “antibody” herein refers to a protein, derived from an immunoglobulin sequence, which is capable of binding to an antigen or a portion thereof. The term antibody includes, but is not limited to, full length antibodies of any class (or isotype), that is, IgA, IgD, IgE, IgG, IgM and/or IgY.

Of particular interest for therapeutic antibodies is the IgG sub-class which in humans is divided into four sub-classes IgG1, IgG2, IgG3 and IgG4, based on the sequence of their heavy chain constant regions. The light chains can be divided into two types, kappa and lambda chains, based on differences in their sequence composition. IgG molecules are composed of two heavy chains, interlinked by two or more disulfide bonds, and two light chains, each attached to a heavy chain by a disulfide bond. An IgG heavy chain may comprise a heavy chain variable domain (V_{H}) and up to three heavy chain constant (C_{H}) domains: C_{H}1, C_{H}2 and C_{H}3. A light chain may comprise a light chain variable domain (V_{L}) and a light chain constant domain (C_{L}). V_{H} and V_{L} regions can be further subdivided into regions of hypervariability, termed complementarity determining regions (CDRs), interspersed with regions that are more conserved, termed framework regions (FR). V_{H} and V_{L} domains are typically composed of three CDRs and four FRs, arranged from amino-terminus to carboxy-terminus in the following order: FR1, CDR1, FR2, CDR2, FR3, CDR3, FR4. The heavy and light chain variable domains containing the hypervariable regions (CDRs) form a structure that is capable of interacting with an antigen, whilst the constant region of an antibody may mediate binding of the antibody to Fc receptors and the first component, C1q, of the C1 complex of the classical complement system.

The term “antigen binding site” or “binding portion” refers to the part of the antibody that enables antigen binding.

The term “antigen-binding fragment” of an antibody refers to a fragment of an antibody that retains the ability to bind to its cognate antigen, such as FVII(a), TLT-1 or another target molecule, as described herein. Examples of antigen-binding fragments include (but is not limited to) Fab, Fab′, Fab₂, Fab′₂, Fv, single-chain Fv (scFv) or a single V_{H} or V_{L} domain.

The term “one-armed antibody” as used herein, refers to a particular type of monovalent antibody fragment composed of an antibody heavy chain, a truncated heavy chain lacking the Fab region, and a single light chain.

The term “monospecific” antibody as used herein, refers to an antibody which is capable of binding to one particular epitope (including but not limited to bivalent antibodies).

The terms “bispecific antibody” and “biAb” herein refers to an antibody which is capable of binding to two different antigens, such as FVII(a) and TLT-1, or two different epitopes on the same antigen.

Bispecific antibodies of the invention are derived from antibodies or antigen-binding fragment thereof. Bispecific antibodies of the inventions may be fusions or conjugates of antibodies and antigen binding fragments of antibodies, such as e.g. Fab, Fab′, Fab2, Fab′2 or scFv. Bispecific antibodies of the invention may also be fusions or conjugates of antibody fragments. A wide range of molecular formats for bispecific antibodies derived from antibodies and antibody fragments are known in the art, see e.g. (Spiess et al.: Molecular Immunology 67, (2015), pp. 95-106) and (Brinkmann and Kontermann: MABS, 9 (2017), pp 182-212).

Bispecific antibodies may be made in a variety of ways described in the art see e.g. (Spiess et al.: Molecular Immunology 67, (2015), pp. 95-106) and (Brinkmann and Kontermann: MABS, 9 (2017), pp 182-212). For example, desired heavy chain pairing can be achieved by engineering the dimerisation interface of the Fc region to promote heterodimerisation. One example hereof is the so-called knob-in-hole mutations where sterically bulky side chains (knobs) are introduced in one Fc matched by sterically small side chains (holes) on the opposite Fc thereby creating steric complementarity promoting heterodimerisation. Other methods for engineered heterodimerisation Fc interfaces are electrostatic complementarity, fusion to non-IgG heterodimerisation domains or utilising the natural Fab-arm exchange phenomenon of human IgG4 to conduct heterodimerisation in vitro. Examples of heterodimerised bispecific antibodies are well described in the literature, e.g. (Klein C, et al.; MAbs. 2012 4, pp 653-663). Special attention has to be paid to the light chains in heterodimeric antibodies. Correct pairing of LCs and HCs can be accomplished by the use of a common light chain. Engineering of the LC/HC interface can be used to promote heterodimerisation or light chain cross-over engineering as in CrossMabs. In vitro re-assembly under mildly reducing conditions of antibodies from two individual IgGs containing appropriate mutations can also be used to generate bispecific antibodies (e.g. Labrijn et al., PNAS, 110 (2013), pp. 5145-5150). Also, the natural Fab-arm exchange method is reported to ensure correct light chains paring.

The term “multispecific” antibody as used herein, refers to an antibody which is capable of binding to two or more different antigens or two or more different epitopes on the same antigen. Multispecific antibodies thus comprise bispecific antibodies.

The antibodies of the invention may be combined with other antibodies and antibody fragments known in the art creating bispecific, trispecific or multispecific antibody molecules.

In one aspect, an antibody of the invention is a chimeric antibody, a human antibody or a humanised antibody. Such antibody can be generated by using, for example, suitable antibody display or immunization platforms or other suitable platforms or methods known in the field.

Furthermore, if the antibody contains a constant region, the constant region or a portion thereof is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

Human antibodies may be isolated from sequence libraries built on selections of human germline sequences, further diversified with natural and synthetic sequence diversity. Human antibodies may be prepared by in vitro immunisation of human lymphocytes followed by transformation of the lymphocytes with Epstein-Barr virus. Human antibodies may be produced by recombinant methods known in the art.

The term “humanised antibody”, as used herein, refers to a human/non-human antibody that contains a sequence (CDR regions or parts thereof) derived from a non-human immunoglobulin. A humanised antibody is, thus, a human immunoglobulin (recipient antibody) in which residues from at least a hypervariable region of the recipient are replaced by residues from a hypervariable region of an antibody from a non-human species (donor antibody) such as from a mouse, rat, rabbit or non-human primate, which have the desired specificity, affinity, sequence composition and functionality. In some instances, framework (FR) residues of the human immunoglobulin are replaced by corresponding non-human residues. An example of such a modification is the introduction of one or more so-called back-mutations, which are typically amino acid residues derived from the donor antibody. Humanisation of an antibody may be carried out using recombinant techniques known to the person skilled in the art (see, e.g., Antibody Engineering, Methods in Molecular Biology, vol. 248, edited by Benny K. Lo). A suitable human recipient framework for both the light and heavy chain variable domain may be identified by, for example, sequence or structural homology. Alternatively, fixed recipient frameworks may be used, e.g., based on knowledge of structure, biophysical and biochemical properties. The recipient frameworks can be germline derived or derived from a mature antibody sequence. CDR regions from the donor antibody can be transferred by CDR grafting. The CDR grafted humanised antibody can be further optimised for e.g. affinity, functionality and biophysical properties by identification of critical framework positions where re-introduction (back-mutation) of the amino acid residue from the donor antibody has beneficial impact on the properties of the humanised antibody. In addition to donor antibody derived back-mutations, the humanised antibody can be engineered by introduction of germline residues in the CDR or framework regions, elimination of immunogenic epitopes, site-directed mutagenesis, affinity maturation, etc. Furthermore, humanised antibodies may comprise residues that are not found in the recipient antibody or in the donor antibody. These modifications are made to further refine antibody performance. The humanised antibody can, optionally, also comprise at least a portion of an immunoglobulin constant region (Fc), typically that of a human immunoglobulin.

The term “chimeric antibody”, as used herein, refers to an antibody comprising portions of antibodies derived from two or more species. For example, the genes encoding such antibody comprise genes encoding variable domains and genes encoding constant domains originated from two different species. For example, the genes encoding variable domains of a mouse monoclonal antibody may be joined to the genes encoding the constant domains of an antibody of human origin.

Antibodies or fragment thereof may be defined in terms of their complementarity-determining regions (CDRs). The term “complementarity-determining region”, when used herein, refers to the regions of an antibody in which amino acid residues involved in antigen-binding are typically situated. The CDRs can be identified as the regions with the highest variability among antibody variable domains. Databases can be used for CDR identification such as the Kabat database, the CDRs e.g. being defined as comprising amino acid residues 24-34 (L1), 50-56 (L2) and 89-97 (L3) of the light-chain variable domain and 31-35 (H1), 50-65 (H2) and 95-102 (H3) in the heavy-chain variable domain; (Kabat et al. 1991; Sequences of Proteins of Immunological Interest, Fifth Edition, U.S. Department of Health and Human Services, NIH Publication No. 91-3242). Typically, the numbering of amino acid residues in this region is performed by the method described in Kabat et al. supra. Phrases such as “Kabat position”, “Kabat residue”, and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. Numbering is according to Kabat only if specifically stated, otherwise numbering is consecutive according to a specified SEQ ID NO.

The term “framework region” or “FR” residues refer to those VH or VL amino acid residues that are not within the CDRs, as defined herein.

An antibody of the invention may comprise a CDR region from one or more of the specific antibodies disclosed herein.

The term “antigen” (Ag) refers to the molecular entity used for immunisation of an immunocompetent vertebrate to produce the antibody (Ab) that recognizes the Ag. Herein, Ag is termed more broadly and is generally intended to include target molecules that are recognized by the Ab.

The present invention encompasses variants of the antibodies, or antigen-binding fragments thereof of the invention, which may comprise 1, 2, 3, 4 or 5 amino acid substitutions and/or deletions and/or insertions in the individual sequences disclosed herein.

“Substitution” variants preferably involve the replacement of one or more amino acids with the same number of amino acids.

The term “epitope”, as used herein, is defined in the context of a molecular interaction between an “antigen binding polypeptide”, such as an antibody (Ab), and its corresponding antigen (Ag). Generally, “epitope” refers to the area or region on an Ag to which an Ab binds, i.e. the area or region in physical contact with the Ab. In the present invention epitopes are determined using an X-ray derived crystal structure, defining the spatial coordinates of a complex between an Ab, such as a Fab fragment, and its Ag. The term epitope is herein, unless otherwise specified or contradicted by context, defined as Ag (here FVII(a) or TLT-1) residues characterized by having a heavy atom (i.e. a non-hydrogen atom) within a distance of 4 Å from a heavy atom in the Fab.

Epitopes described at the amino acid level, e.g. determined from an X-ray structure, are said to be identical if they contain the same set of amino acid residues. Epitopes are said to overlap if at least one amino acid residue is shared by the epitopes. Epitopes are said to be separate (unique) if no amino acid residue is shared by the epitopes.

The definition of the term “paratope” is derived from the above definition of “epitope” by reversing the perspective. Thus, the term “paratope” refers to the area or region on the antibody, or fragment thereof to which an antigen binds, i.e. to which it makes physical contact to the antigen. The term paratope is herein, unless otherwise specified or contradicted by context, defined as Ab residues characterized by having a heavy atom (i.e. a non-hydrogen atom) within a distance of 4 Å from a heavy atom in FVII(a) or TLT-1. Epitopes on an antigen may comprise one or more hot-spot residues, i.e. residues which are particularly important for the interaction with the cognate antibody, and where interactions mediated by the side chain of said hot-spot residue contribute significantly to the binding energy for the antibody/antigen interaction (Peng et al. PNAS 111 (2014), E2656-E2665). Hot-spot residues can be identified by testing variants of the antigen, where single epitope residues have been substituted by e.g. alanine, for binding to the cognate antibody. If substitution of an epitope residue with alanine has a strong impact on binding to the antibody, said epitope residue is considered a hot-spot residue, and therefore of particular importance for binding of the antibody to the antigen.

Antibodies that bind to the same antigen can be characterised with respect to their ability to bind to their common antigen simultaneously and may be subjected to “competition binding” /“binning”. In the present context, the term “binning” refers to a method of grouping antibodies that bind to the same antigen. “Binning” of antibodies may be based on competition binding of two antibodies to their common antigen in assays based on standard techniques. An antibody's “bin” is defined using a reference antibody. If a second antibody is unable to bind to an antigen at the same time as the reference antibody, the second antibody is said to belong to the same “bin” as the reference antibody. In this case, the reference and the second antibody competitively bind the same part of an antigen and are coined “competing antibodies”. If a second antibody is capable of binding to an antigen at the same time as the reference antibody, the second antibody is said to belong to a separate “bin”. In this case, the reference and the second antibody do not competitively bind the same part of an antigen and are coined “non-competing antibodies”.

Competition assays for determining whether an antibody competes for binding with an anti-FVII(a) or anti-TLT-1 antibody disclosed herein are known in the art. Exemplary competition assays include immunoassays (e.g., ELISA assays, RIA assays), surface plasmon resonance analysis (e.g. using a BIAcore™ instrument), biolayer interferometry (ForteBio®) and flow cytometry.

Typically, a competition assay involves the use of an antigen bound to a solid surface or expressed on a cell surface, a test FVII- or FVIIa binding antibody and a reference antibody. The reference antibody is labelled and the test antibody is unlabelled. Competitive inhibition is measured by determining the amount of labelled reference antibody bound to the solid surface or cells in the presence of the test antibody. Usually the test antibody is present in excess (e.g., 1, 5, 10, 20, 100, 1000, 10000 or 100000-fold). Antibodies identified as being competitive in the competition assay (i.e., competing antibodies) include antibodies binding to the same epitope, or overlapping epitopes, as the reference antibody, and antibodies binding to an adjacent epitope sufficiently proximal to the epitope bound by the reference antibody for steric hindrance to occur.

In an exemplary competition assay, a reference anti-FVII or anti-FVIIa antibody is biotinylated using commercially available reagents. The biotinylated reference antibody is mixed with serial dilutions of the test antibody or unlabelled reference antibody (self-competition control) resulting in a mixture of various molar ratios (e.g., 1, 5, 10, 20, 100, 1000, 10000 or 100000-fold) of test antibody (or unlabelled reference antibody) to labelled reference antibody. The antibody mixture is added to a FVII or FVIIa polypeptide coated-ELISA plate. The plate is then washed, and horseradish peroxidase (HRP)-strepavidin is added to the plate as the detection reagent. The amount of labelled reference antibody bound to the target antigen is detected following addition of a chromogenic substrate (e.g., TMB (3,3′,5,5′-tetramethylbenzidine) or ABTS (2,2″-azino-di-(3-ethylbenzthiazoline-6-sulfonate)), which are known in the art. Optical density readings (OD units) are made using a spectrometer (e.g. SpectraMax® M2 spectrometer (Molecular Devices)). The response (OD units) corresponding to zero percent inhibition is determined from wells without any competing antibody. The response (OD units) corresponding to 100% inhibition, i.e. the assay background, is determined from wells without any labelled reference antibody or test antibody. Percent inhibition of labelled reference antibody to FVII or FVIIa by the test antibody (or the unlabelled reference antibody) at each concentration is calculated as follows: % inhibition=(1−(OD units−100% inhibition)/(0% inhibition−100% inhibition))*100.

The person skilled in the art will understand that similar assays may be performed to determine if two or more anti-TLT-1 antibodies shares a binding region, a bin and/or competitively binds the antigen. Persons skilled in the art will also appreciate that the competition assay can be performed using various detection systems known in the art. A test antibody competes with the reference antibody for binding to the antigen if an excess of one antibody (e.g., 1, 5, 10, 20, 100, 1000, 10000 or 100000-fold) inhibits binding of the other antibody, e.g., by at least 50%, 75%, 90%, 95% or 99%, as measured in a competitive binding assay.

Unless otherwise indicated competition is determined using a competitive ELISA assay as described above and provided in Example 7 and Example 32.

The term “binding affinity” is herein used as a measure of the strength of a non-covalent interaction between two molecules, e.g. an antibody, or fragment thereof, and an antigen. The term “binding affinity” is used to describe monovalent interactions. Binding affinity between two molecules, e.g. an antibody, or fragment thereof, and an antigen, through a monovalent interaction may be quantified by determining the equilibrium dissociation constant (K_{D}). K_{D} can be determined by measurement of the kinetics of complex formation and dissociation, e.g. by the Surface Plasmon Resonance (SPR) as done in Examples 6 and 16 or other methods known in the art. The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ.

Following the above definition, binding affinities associated with different molecular interactions, such as comparison of the binding affinity of different antibodies for a given antigen, may be compared by comparison of the K_{D} values for the individual antibody/antigen complexes.

The K_{D} of an antibody of the invention for its target may be less than 1 pM such as less than 10 pM, such as less than 100 pM, such as less than 200 pM, such as less than 400 pM, such as less than 600 pM, such as less than 1 nM, such as less than 5 nM, such as less than 10 nM, such as less than 20 nM, such as less than 50 nM, such as less than 100 nM, such as less than 200 nM, such as less than 400 nM, such as less than 600 nM, such as less than 800 nM.

In one such embodiment the antibody is a bispecific antibody comprising an anti-FVII(a) arm with a K_{D} towards FVIIa of less than 1 pM such as less than 10 pM, such as less than 100 pM, such as less than 200 pM, such as less than 400 pM, such as less than 600 pM, such as less than 1 nM, such as less than 5 nM, such as less than 10 nM, such as less than 20 nM, such as less than 50 nM, such as less than 100 nM, such as less than 200 nM, such as less than 400 nM, such as less than 600 nM, such as less than 800 nM, and a second anti-TLT-1 arm with a K_{D} towards TLT-1 of less than 1 pM such as less than 10 pM, such as less than 100 pM, such as less than 200 pM, such as less than 400 pM, such as less than 600 pM, such as less than 1 nM, such as less than 5 nM, such as less than 10 nM, such as less than 20 nM, such as less than 50 nM, such as less than 100 nM, such as less than 200 nM, such as less than 400 nM, such as less than 600 nM, such as less than 800 nM.

The term “identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. In the present invention similarity and identity were determined using Needleman (Needleman et al. J. Mol. Biol. 1970; 48:443-453) from EMBOSS-6.6.0 using the parameters 10 and 0.5 for gaps opening and extensions, respectively (gapopen=10, gapextend=0.5).

The fragment crystallisable region (“Fc region” /“Fc domain”) of an antibody is the C-terminal region of an antibody, which comprises the hinge and the constant C_{H}2 and C_{H}3 domains.

Antibodies of the invention may comprise an Fc region which may have a wild-type amino acid sequence, or it may comprise amino acid substitutions which modulates the effector functions of the antibody, see e.g. (Wang et al.: Protein Cell. 9 (2018), pp. 63-73). Specific examples of Fc variants with modified effector functions are variants where binding to Fcγ receptors has been reduced. One specific example of such a variant is IgG1 comprising the substitutions L234A, L235E, G237A, A330S and P331S (residue numbering according to the EU index) with decreased affinity for certain Fcγ receptors and C1q.

### Bispecific Molecules

The term “bispecific molecules” herein refers to molecules capable of binding to different targets, such as FVII(a) and TLT-1. The binding moieties of the bispecific molecule may be derived from an antibody or may be of non-antibody origin. One particular example of a bispecific molecule is a bispecific antibody.

In an aspect of the invention, the bispecific molecule comprises a first antigen binding site capable of binding Factor VII(a), and a second antigen binding site capable of binding TREM-like Transcript 1 (TLT-1).

Bispecific molecules of the invention may comprise non-antibody derived binding moieties, also referred to as alternative scaffolds. Bispecific molecules of the invention may be fusions or conjugates of alternative scaffolds. Bispecific molecules of the invention may be fusions or conjugates of antibodies and alternative scaffolds. Bispecific molecules of the invention may also be fusions or conjugates of antibody fragments and alternative scaffolds.

A large number and variety of alternative scaffolds are known in the art, see e.g. (Simeon and Chen: Protein Cell 9 (2018), pp. 3-14), (Könning and Kolmar: Microbial Cell Factories (2018), pp. 17-32), and (Nygren and Skerra: Journal of Immunological Methods 290 (2004), pp 3-28).

Specific examples of alternative scaffolds are Adnectins, Affilins, Anticalins, Avimers, Atrimers, FN3 scaffolds, Fynomers, OBodies, Kringle domains, Kunitz domains, Knottins, Affibodies, DARPins, Bicyclic peptides and Cys-knots.

### Factor VII(a)

The terms “Factor VII” and “FVII” herein refer to the zymogen of coagulation factor VII. The terms “Factor VIIa” and “FVIIa” herein refer to activated coagulation factor VII, which is a serine protease. The terms “Factor VII(a)” and “FVII(a)” herein encompass the uncleaved zymogen, Factor VII (FVII), as well as the cleaved and thus activated protease, Factor VIIa (FVIIa). The terms “Factor VII(a)” and “FVII(a)” herein include natural allelic variants of FVII(a) that may exist. One wild type human Factor VII(a) sequence is provided in SEQ ID NO: 1.

Wild type human coagulation Factor VII(a) (SEQ ID
NO. 1):
ANAFLEELRPGSLERECKEEQCSFEEAREIFKDAERTKLFWISYSDGDQ
CASSPCQNGGSCKDQLQSYICFCLPAFEGRNCETHKDDQLICVNENGGC
EQYCSDHIGTKRSCRCHEGYSLLADGVSCTPTVEYPCGKIPILEKRNAS
KPQGRIVGGKVCPKGECPWQVLLLVNGAQLCGGTLINTIWVVSAAHCFD
KIKNWRNLIAVLGEHDLSEHDGDEQSRRVAQVIIPSTYVPGTTNHDIAL
LRLHQPVVLTDHVVPLCLPERTFSERTLAFVRFSLVSGWGQLLDRGATA
LELMVLNVPRLMTQDCLQQSRKVGDSPNITEYMFCAGYSDGSKDSCKGD
SGGPHATHYRGTWYLTGIVSWGQGCATVGHFGVYTRVSQYIEWLQKLMR
SEPRPGVLLRAPFP

Wild type FVII(a) consists of 406 amino acid residues and is composed of four domains. There is an N-terminal gamma-carboxyglutamic acid-rich (Gla) domain, where ten glutamate residues (highlighted in bold in the sequence above) may be gamma-carboxylated. The gla domain is followed by two epidermal growth factor (EGF)-like domains and a C-terminal serine protease domain. Both FVII and FVIIa are present in circulation, but FVIIa only at low abundance (approximately 1% of the total FVII(a) pool; Morrissey J H, Broze Jr G J. Tissue factor and the initiation and regulation (TFPI) of coagulation. In: Marder V J, Aird W C, Bennett J S, Schulman S, White II G C, editors. Hemostasis and thrombosis: basic principles and clinical practice. 6th ed. Wolters Kluwer & Lippincott Williams & Wilkins: Philadelphia; 2013. p. 163-78). FVII can be activated to FVIIa by proteolytic cleavage between residues Arg152 and Ile153, resulting in the two-chain FVIIa molecule consisting of a light chain and a heavy chain. The two chains in FVIIa are tethered by a disulfide bond. The light chain contains the Gla and EGF-like domains, and the heavy chain the protease domain. FVIIa requires binding to its cell-surface co-factor tissue factor (TF) to reach its full biological activity.

The predicted full-length cynomolgus monkey (Macaca fascicularis) FVII isoform X1 consists of 406 amino acids with the NCBI reference sequence ID XP_015295043.1. The term “cFVIIa-chimera” herein refers to the chimeric cynomolgus monkey FVIIa construct. The amino acid sequence of cFVIIa-chimera is such that the Gla and the first EGF-like domains (amino acids 1-88 when aligned with the human FVIIa sequence) are comprised of the human FVII sequence (Uniprot ID P08709); whereas, the second EGF-like and the protease domains (amino acids 89-406 when aligned with the human FVIIa sequence) are comprised of the cynomolgus monkey FVII isoform X1 sequence (NCBI reference sequence ID XP_015295043.1)

The active half-life of recombinant FVIIa (and endogenous FVIIa) in humans is about 2 to 3 hours, when dosed intravenously. Factor VII(a) may be endogenous, plasma-derived or recombinantly produced, using well known methods of production and purification. The degree and location of glycosylation, gamma-carboxylation and other post-translational modifications may vary depending on the chosen host cell and its growth conditions.

Factor VIIa may be found in different conformations. Factor VIIa circulates in the blood in an inactive conformation or inactive form. This conformation does not have catalytic activity. FVIIa may also be found in an active conformation or active form, herein also referred to as fully active or fully activated FVIIa. The term FVIIa encompasses FVIIa in the inactive form or conformation and FVIIa in the active form or conformation. For example, the active conformation of FVIIa may comprise a complex between FVIIa and tissue factor (FVIIa/TF), e.g. in the form of FVIIa/sTF (1-219), where sTF (1-219) is a truncated and soluble form of tissue factor, or the active conformation of FVIIa may comprise active-site inhibited FVIIa (FVIIai). FVIIai is a catalytically inert form of FVIIa that may be produced by the treatment of FVIIa with Dansyl-Glu-Gly-Arg chloromethyl ketone or Phe-Phe-Arg-chloromethyl ketone (FFR-chloromethyl ketone) (Wildgoose et al (1990) Biochemistry 29:3413-3420 and Sørensen et al (1997) J Biol Chem 272:11863-11868). FVIIai retains its affinity for TF and is thought to adopt the same conformation as that induced in FVIIa by TF binding. FVIIai thus has an activated conformation and binding of a test compound to FVIIai is indicative that the test compound will also bind to an activated form of wild-type FVIIa.

The target molecule of an anti-FVII(a) antibody may be any FVII(a) molecule as described herein.

### TREM-Like Transcript 1 (TLT-1)

Triggering receptors expressed on myeloid cells (TREMs) have a well-established role in the biology of various myeloid lineages, playing important roles in the regulation of innate and adaptive immunity. TREM-like transcript (TLT)-1 belongs to this family of proteins, though the TLT-1 gene is expressed only in a single lineage, namely megakaryocytes and thrombocytes (platelets) and is exclusively found in the alpha-granules of megakaryocytes and platelets. TLT-1 is a transmembrane protein that is exposed on the surface of activated platelets upon alpha-granule release. To date, TLT-1 has not been found on the surface of resting platelets or on the surface of any other cell type.

TLT-1 contains an extracellular globular head, a stalk region, a transmembrane domain and an intracellular domain containing immunoreceptor tyrosine-based inhibitory motifs (Washington et al. Blood, 2002; 100:3822-3824). The extracellular globular head of human TLT-1 (hTLT-1) is a single immunoglobulin-like (Ig-like) domain. It is connected to the platelet membrane by a 37-amino acid linker region referred to as the stalk (Gattis et al., Jour Biol Chem, 2006, 281, 19, 13396-13403).

The putative transmembrane segment of hTLT-1 is 20 amino acids long. TLT-1 also has a cytoplasmic Immune-receptor Tyrosine-based Inhibitory-Motif (ITIM), which may function as an intracellular signal transduction motif.

A fraction of TLT-1 is shed upon platelet activation, forming a soluble form (sTLT-1) (Gattis et al., Jour Biol Chem, 2006, 281, 19, 13396-13403). The cleavage site is situated in close proximity to the platelet membrane. A shorter isoform of TLT-1 with a truncated intracellular domain also exists in platelets.

TLT-1 is involved in regulating coagulation and possibly also inflammation at site of an injury. TLT-1 plays a role in platelet aggregation in response to sub-optimal concentration of some platelet agonists (Giomarelli et al, Thromb Haemost 2007; 97:955-963.). The best described ligand to TLT-1 is fibrinogen (Washington et al. J Clin Invest 2009; 119:1489-3824.). Recently it was shown that TLT-1 also binds von Willebrand Factor (Doerr A et al, abstract PB359 at International Society of Thrombosis and Haemostasis 2019). sTLT-1 is suggested to have a role in haemorrhage related to sepsis by dampening leukocyte activation and modulating platelet-neutrophil cross-talk (Derive, J Immunol 2012, 188:5585-5592).

In terms of the current invention, TLT-1 may be from any vertebrate, such as any mammal, such as a rodent (such as a mouse, rat or guinea pig), a lagomorph (such as a rabbit), an artiodactyl (such as a pig, cow, sheep or camel) or a primate (such as a monkey or human being). TLT-1 is, preferably, human TLT-1. TLT-1 may be translated from any naturally occurring genotype or allele that gives rise to a functional TLT-1 protein. A non-limiting example of one human TLT-1 is the polypeptide sequence of SEQ ID NO: 2.

The target molecule of a TLT-1 antibody may be any TLT-1 molecule as described herein.

### Anti-FVII(a) Antibodies

The term “anti-FVII(a) antibody” herein refers to antibody having FVII(a) as its target. The anti-FVII(a) antibody is capable of binding to FVII(a) molecules as described herein; including, but not limited to endogenous FVII(a) as found in human plasma, exogenous FVII(a), such as recombinant wild type human FVII(a), and endogenous FVII(a) as found in animal plasma, such as in the rabbit, mouse, rat, dog or monkey.

An anti-FVII(a) antibody” could be a monoclonal, monospecific antibody having FVII(a) as its target. A monospecific anti-FVII(a) antibody typically comprises two identical antigen binding sites that bind FVII(a); non-limiting examples being monoclonal IgG4 anti-FVII(a) antibodies.

Suitable anti-FVII(a) antibodies include, without limitation, any one of anti-FVII(a) antibodies shown in Table 3.

TABLE 3

| | | VL | VH | Constant |
|---|---|---|---|---|
| Antibody | Target | SEQ ID NO | SEQ ID NO | domain |
| mAb0522 | FVII(a) | 846 | 850 | K |
| Fab0883 | FVII(a) | 814 | 818 | G |
| mAb0005 | FVII(a) | 750 | 754 | M |
| mAb0004 | FVII(a) | 14 | 18 | M |
| mAb0013 | FVII(a) | 46 | 50 | M |
| mAb0018 | FVII(a) | 62 | 66 | M |
| mAb0544 | FVII(a) | 694 | 698 | M |
| mAb0552 | FVII(a) | 702 | 706 | M |
| mAb0001 | FVII(a) | 710 | 714 | M |
| mAb0007 | FVII(a) | 718 | 722 | M |
| mAb0578 | FVII(a) | 726 | 730 | M |
| mAb0701 | FVII(a) | 734 | 738 | M |
| mAb0587 | FVII(a) | 742 | 746 | M |

The anti-FVII(a) antibody may be capable of competing, with any one of the antibodies shown in Table 3, for binding to FVII(a). Whether or not an anti-FVII(a) antibody competes with any one of the antibodies shown in Table 3, for binding to FVII(a), may be determined using methods (that is, competitive binding assays) that are well-known, such as surface plasmon resonance (SPR), ELISA or flow cytometry. Example 7 describes how competition binding to FVII(a) may be determined using a competitive ELISA.

In one embodiment, the anti-FVII(a) antibody binds to FVII(a) with a high affinity. A anti-FVII(a) antibody may have a K_{D} for its target of less than 1 pM, such as less than 10 pM, such as less than 100 pM, such as less than 200 pM, such as less than 400 pM, such as less than 600 pM, such as less than 1 nM, such as less than 5 nM, such as less than 10 nM, such as less than 20 nM, such as less than 50 nM, such as less than 100 nM, such as less than 200 nM, such as less than 400 nM, such as less than 600 nM, such as less than 800 nM. In vivo, the high-affinity anti-FVII(a) antibody reduces the clearance of FVII(a) by forming a complex with it. Thereby, it prolongs the half-life of FVII(a) and causes it to accumulate in the circulation. In this manner, the antibody can provide an elevated steady-state concentration of FVII(a).

In one embodiment, the anti-FVII(a) antibody does not interfere with the biological function of FVII(a). In an embodiment, the anti-FVII(a) antibody does not prevent endogenous FVII from being activated to FVIIa. For example, the anti-FVII(a) antibodies do not interfere with the ability of FVII to convert into FVIIa (i.e., to auto-activate), whilst bound to TF (as described in Example 12). It is also preferable that the anti-FVII(a) antibody does not prevent FVII(a) from forming the so-called initiation complex with tissue factor (TF) and to activate Factor X (FX) in a TF-dependent or independent manner (as described in Example 10). In an embodiment, the anti-FVII(a) antibody does not compete with FVII(a) substrates or co-factors. However, the anti-FVII(a) antibody might compete with inhibitors of FVIIa, such as antithrombin (AT) and alpha-2-macroglobulin (as described in Example 11).

### Anti-TLT-1 Antibodies

The term “anti-TLT-1 antibody” herein refers to antibody having TLT-1 as its target. The anti-TLT-1 antibody is capable of binding to TLT-1 molecules as described herein. An TLT-1 antibody” could be a monoclonal, monospecific antibody.

Suitable anti-TLT-1 antibodies include, without limitation, the anti-TLT-1 antibodies shown in Table 4

TABLE 4

| | | VL | VH | Constant |
|---|---|---|---|---|
| Antibody | Target | SEQ ID NO | SEQ ID NO | domain |
| mAb0524 | TLT-1 | 854 | 858 | J |
| mAb0012 | TLT-1 | 862 | 866 | A |
| mAb0082 | TLT-1 | 902 | 906 | A |
| mAb1038 | TLT-1 | 910 | 914 | A |
| mAb1047 | TLT-1 | 918 | 922 | A |
| mAb1049 | TLT-1 | 926 | 930 | A |
| mAb0061 | TLT-1 | 886 | 890 | A |
| mAb0023 | TLT-1 | 870 | 874 | A |
| mAb0051 | TLT-1 | 878 | 882 | A |
| mAb0062 | TLT-1 | 894 | 898 | A |

The anti-TLT-1 antibody may be capable of competing, with any one of the antibodies shown in Table 4 for binding to TLT-1. Whether or not an anti-TLT-1 antibody competes with any one of the antibodies shown in Table 4, for binding to TLT-1, may be determined using methods that are well-known (that is, competitive binding assays), such as surface plasmon resonance (SPR), ELISA or flow cytometry. Competition binding to TLT-1 may be determined using a competitive ELISA. Example 32 describes how competition binding to TLT-1 may be determined using a competitive ELISA.

A anti-TLT-1 antibody may have a K_{D} for its target of less than 1 pM, such as less than 10 pM, such as less than 100 pM, such as less than 200 pM, such as less than 400 pM, such as less than 600 pM, such as less than 1 nM, such as less than 5 nM, such as less than 10 nM, such as less than 20 nM, such as less than 50 nM, such as less than 100 nM, such as less than 200 nM, such as less than 400 nM, such as less than 600 nM, such as less than 800 nM.

It is preferable that the anti-TLT-1 antibody does not interfere with functions of TLT-1 and—in particular—does not inhibit platelet aggregation.

In one preferred embodiment, anti-TLT-1 antibodies are capable of binding to TLT-1 without interfering with platelet aggregation.

In another preferred embodiment, anti-TLT-1 antibodies are capable of binding to TLT-1 without competing with fibrinogen for binding to TLT-1.

In another preferred embodiment, TLT-1 antibodies do not interfere with the shedding of TLT-1.

It is preferable that anti-TLT-1 antibodies do not bind to, or demonstrate little affinity for, any other triggering receptor expressed on myeloid cells (TREM) than TLT-1, or any other receptor on the resting or activated platelet.

In one embodiment, the anti-TLT-1 antibody binds to the stalk of TLT-1.

### Bispecific Anti-FVII(a)/Anti-TLT-1 Antibodies

The bispecific antibodies of the invention comprise a first antigen binding site capable of binding FVII(a), and a second antigen binding site capable of binding TLT-1.

### Anti-FVII(a) Antigen Binding Site

In an aspect, the bispecific antibodies of the invention comprise a first antigen binding site capable of binding FVII(a).

In some embodiments of the invention, the first antigen-binding site of the bispecific antibody competes for binding to FVII(a) with any one of the anti-FVII(a) antibodies identified in Table 3; have an epitope identical to any one of the antibodies identified in Table 3; have CDR regions identical to any one of the antibodies identified in Table 3; and have VL and VH regions identical to any one of the antibodies identified in Table 3.

### Anti-TLT-1 Antigen Binding Site

In an aspect, the bispecific antibodies of the invention comprise a second antigen binding site capable of binding TLT-1.

In some embodiments of the invention, the second antigen-binding site of the bispecific antibody competes for binding to TLT-1 with any one of the anti-TLT-1 antibodies identified in Table 4; have an epitope identical to any one of the antibodies identified in Table 4; have CDR regions identical to any one of the antibodies identified in Table 4; and have VL and VH regions identical to any one of the antibodies identified in Table 4.

### Modified Effector Functions

Bispecific antibodies of the invention may comprise an Fc region which may have a wild-type amino acid sequence, or it may comprise amino acid substitutions which modulates the effector functions of the antibody, see e.g. (Wang et al.: Protein Cell. 9 (2018), pp. 63-73). Specific examples of Fc variants with modified effector functions are variants where binding to Fcγ receptors has been reduced. One specific example of such a variant is IgG1 comprising the substitutions L234A, L235E, G237A, A330S and P331S (residue numbering according to the EU index) with decreased affinity for certain Fcγ receptors and C1q.

A desired property of the bispecific antibodies of the invention is a long in vivo half-life. Bispecific antibodies comprising a Fc-region may be recycled and rescued via the FcRn receptor which, in turn, may result in the desired long half-life. For bispecific antibodies of the invention lacking a Fc-region half-life may be extended by other means. Different methods and principles for obtaining a prolonged half-life of polypeptides and antibodies are known in the art, see e.g. Kontermann: Expert Opinion on Biological Therapy, 16 (2016), pp. 903-915) and references therein.

In addition to Fc-based half-life extension of polypeptides and antibodies, fusion or conjugation to albumin or albumin variants have proven effective in half-life prolongation. Another approach is attachment of polymers, such as XTEN or PEG (insert reference). Furthermore, prolonged in vivo half-life can be obtained by attachment of an albumin binding moiety, see e.g. Tan et al.: Current Pharmaceutical Design 24 (2018), pp. 4932-4946; Kontermann: Expert Opinion on Biological Therapy, 16 (2016), pp. 903-915) and Kontermann: Current Opinion in Biotechnology 22 (2011), pp 868-876).

### Functional Features

By binding to FVII(a), the bispecific antibodies of the invention may prolong the active circulatory half-life of FVIIa present in circulation; by binding to TLT-1, the bispecific antibodies and bound FVII(a) is directed to the surface of the activated platelet. This, in turn, leads to increased accumulation of FVIIa on the activated platelet and thus enhanced FVIIa pro-coagulant activity at the site of vascular injury. The bispecific antibodies described herein are thus capable of endowing the endogenous pool of FVII(a) with improved pharmacokinetic (PK) and pharmacodynamic (PD) properties.

In humans, the active half-life of administered recombinant FVIIa is known to be about 2-3 hours. The short half-life of recombinant FVIIa is believed to be due to the involvement of several mechanisms, including inhibition by antithrombin III (AT), inhibition by alpha-2-macroglobulin (α2M) and renal clearance. Similar mechanisms are believed to hold true for endogenous FVIIa, thereby endowing it with a similar short half-life.

In order to prolong the active half-life of FVIIa, including the half-life of endogenous FVIIa, the bispecific antibodies described herein aim to intercept one or more of these clearance mechanisms by means of its anti-FVII arm or so-called “first antigen-binding site”, without loss of endogenous FVIIa activity. The Fc portion of the biAb:FVIIa complex will, in the endosome and via binding to FcRn, mediate recycling of the complex and rescue it from degradation. Furthermore, a high-affinity anti-FVII arm of the bispecific antibodies protect endogenous FVIIa from α2M inhibition and renal clearance by virtue of the increased molecular size of the biAb:FVIIa complex, relative to the size of the free endogenous FVIIa. The anti-TLT-1 arm of the bispecific antibodies selectively localize the protracted endogenous FVIIa to activated platelets. This localization of FVIIa to activated platelets potentiates the FVIIa activity without increasing its susceptibility to AT inhibition.

The bispecific antibodies of the invention may increase the mean residence time (MRT) of endogenous or exogenous FVII(a). It is preferable that the bispecific antibodies are capable of potentiating the activity of FVII(a) in vivo.

### Mean Residence Time

The mean residence time (MRT) is the average time a molecule stays in the body, available for therapeutic activity. The MRT is calculated according to equation 1 as a function of volume of distribution at steady-state (Vss) divided by systemic clearance (CL).

$$M R T = V s s / C L \qquad Eq . 1$$

The results are expressed in time. MRT is correlated with effective plasma half-life (t_{1/2}) according to equation 2.

$$M R T = ln ( 2 ) * {t}_{1 / 2} \qquad Eq . 2$$

The ability of an antibody to increase MRT of FVII(a) may be determined by well-known methods, such as such as those described in Pharmacokinetic and Pharmacodynamic Data Analysis: Concepts & Applications (Gabrielsson and Weiner). For example, by analysis of plasma concentration or activity profiles of FVII(a) after IV or SC administration into experimental animals, e.g. mice, rats or monkeys. The ability of an antibody to increase the functional MRT of FVII(a) may be determined by analysis of plasma activity profiles of FVII(a) as measured with assays such as, but not limited to, the FVIIa activity assay described in Example 8.

The ability of an antibody to increase MRT and the functional MRT of FVII(a) may, for example, be determined as described in Examples 9, and 18.

In some embodiments, the bispecific antibodies of the invention are capable of increasing the MRT of FVII(a) by at least 2-fold, at least 3-fold, at least 5-fold, at least 10-fold, at least 20-fold, at least 30-fold, or at least 40-fold compared with administration of FVII(a) in the absence of an antibody of the invention (FVII(a) polypeptide alone).

In some embodiments, bispecific antibodies of the invention are capable of increasing the functional MRT of FVII(a), as measured with a FVIIa activity assay described in Example 8, by at least 2-fold, at least 3-fold, at least 5-fold, at least 10-fold, at least 20-fold, at least 30-fold, or at least 40-fold compared with administration of FVII(a) in the absence of the bispecific antibody of the invention.

### Accumulation of Endogenous FVIIa

The ability of the bispecific antibodies of the invention to increase the level of circulating endogenous functionally active FVIIa may, for example, be determined by measuring the level of endogenous FVIIa before and after administration of the antibody into experimental animals, e.g. mice, rats or monkeys, measured with assays such as, but not limited to, the FVIIa activity assay described in Example 8.

The ability of an antibody to increase the level of circulating endogenous functionally active FVIIa may, for example, be determined as described in Examples 27 and 28.

In some embodiments, the bispecific antibodies of the invention are capable of increasing the level of the circulating endogenous FVIIa by at least 2-fold, at least 4-fold, at least 10-fold, at least 20-fold, at least 40-fold, at least 80-fold, at least 160-fold, at least 320-fold, at least 640-fold compared to the level of circulating endogenous FVIIa in the absence of administered bispecific antibody.

### TLT-1- and TF-Independent Thrombin Generation

In an aspect, the bispecific antibodies of the invention are capable of maintaining or increasing the TLT-1- and TF-independent ability of Factor VIIa to generate thrombin.

The ability of an antibody to increase thrombin generation of a FVII(a) polypeptide may be determined by methods well-known in the art, such as, e.g., a thrombin generation assay as described in Example 5. In this assay, in this assay thrombin generation is measured in hemophilia A-induced human plasma in the presence of phospholipids, 25 nM FVIIa, and an antibody at a concentration approaching saturation of added FVIIa. Saturation is approached when >90% of FVIIa is bound by antibody according to the measured dissociation constant determined for the FVIIa-antibody interaction by e.g. SPR as exemplified in Example 6. Based on the ratio of peak thrombin formation in the presence and absence of antibodies, antibodies are categorized as stimulatory (>120%), inhibitory (<90%), or neutral (90-120%).

In some embodiments, the bispecific antibodies of the invention are capable of maintaining (neutral)/increasing (stimulatory) the ability of the Factor VII(a) polypeptide to generate thrombin as measured in a thrombin generation assay compared with FVII(a) in the absence of the antibody.

In some embodiments, the bispecific antibodies of the invention are capable of increasing the ability of Factor VII(a) to generate thrombin as measured in a thrombin generation assay to at least 20% compared with FVII(a) in the absence of the antibody.

Inhibition by Antithrombin and/or Alpha-2-Macroglobulin

In an aspect, the bispecific antibodies of the invention are capable of reducing the susceptibility of FVIIa to inhibition by antithrombin (AT) and/or alpha-2-macroglobulin.

The ability of an antibody of reducing the inhibition of FVIIa by antithrombin (AT) and/or alpha-2-macroglobulin may be determined by methods well-known in the art such as described in Example 5 and 11.

In some embodiments, the bispecific antibodies of the invention are capable of reducing the inhibition of FVIIa by antithrombin (AT) and/or alpha-2-macroglobulin compared with the inhibition of FVIIa in the absence of the antibody.

### TLT-1-Dependent FXa Generation (Stimulatory Activity Assay)

In an aspect, the bispecific antibodies of the invention are capable of maintaining or increasing the ability of a FVIIa polypeptide to promote FX activation in the presence of a TLT-1-containing procoagulant membrane surface.

The ability of a bispecific antibody of the invention to increase the ability of a FVIIa polypeptide to promote FX activation may be determined by methods well-known in the art, such as, e.g. a TLT-1-dependent stimulatory activity assay as described in Example 21. In this assay, FX activation is measured in the presence of FX (150 nM), FVIIa (2.5 nM), phospolipid membranes containing TLT-1 (4 nM), and an anti-FVII(a)/anti-TLT-1 bispecific antibody. The so-called stimulatory activity of a bispecific antibody (expressed as fold increase), is the amount of generated FXa in the presence of 100 nM bispecific antibody relative to the amount generated by FVIIa in the absence of bispecific antibody.

In some embodiments, the stimulatory activities of the bispecific antibodies of the invention are at least 2-fold, at least 5-fold, at least 10-fold, at least 20-fold, at least 30-fold, at least 40-fold, at least 60-fold, at least 80-fold, at least 100-fold, or at least 150-fold.

### TLT-1-Dependent Whole Blood Clot Formation

In an aspect, the bispecific antibodies of the invention are capable of promoting clot formation similar to or better than a therapeutic effective concentration of recombinant FVIIa under hemophilia A conditions.

The ability of a bispecific antibody to improve whole blood clot formation may be determined by methods well-known in the art, such as, e.g. a thromboelastography assay as described in Example 29. In this assay, clot formation is measured in human hemophilia-A induced whole blood to which bispecific antibody is added together with FVII, FVIIa, and FVIIa:AT at concentrations mimicking the in vivo steady state plasma levels of the corresponding endogenous factors upon repeated administration of the bispecific antibody as described in Examples 27, 28, and 29. Coagulation is induced by addition of the PAR1 agonist peptide SFLLRN and calcium. The clot time under these conditions are compared to the clot time achieved by addition of 25 nM FVIIa in the absence of antibody.

In some embodiments, bispecific antibodies of the invention are capable of reducing the clot time in human haemophilia A-induced whole blood to a level similar to or below that achieved by addition of 25 nM FVIIa. In some embodiments, bispecific antibodies of the invention are capable of reducing the clot time in human haemophilia A-induced whole blood to a level similar to or below that achieved by addition of 2 nM FVIIa, 4 nM FVIIa, 6 nM FVIIa, 8 nM FVIIa, 10 nM FVIIa, 12 nM FVIIa, 16 nM FVIIa or 20 nM FVIIa.

### Pharmaceutical Formulations

In an aspect, the present invention provides compositions and formulations comprising the bispecific antibodies described herein. For example, the invention provides a pharmaceutical composition that comprises a bispecific antibody formulated together with a pharmaceutically acceptable carrier.

In an embodiment of the invention, the pharmaceutical formulation comprises a bispecific antibody present in a concentration from 80 mg/ml to 200 mg/ml, such as 100-180 mg/ml, and wherein said formulation has a pH from 2.0 to 10.0. The formulation may further comprise one or more of a buffer system, a preservative, a tonicity agent, a chelating agent, a stabilizer or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19^{th} edition, 1995.

In one embodiment, the pharmaceutical formulation is an aqueous formulation. Such a formulation is typically a solution or a suspension, but may also include colloids, dispersions, emulsions and multi-phase materials. The term “aqueous formulation” is defined as a formulation comprising at least 50% w/w water. Likewise, the term “aqueous solution” is defined as a solution comprising at least 50% w/w water and the term “aqueous suspension” is defined as a suspension comprising at least 50% w/w water.

In another embodiment, the pharmaceutical formulation is a freeze-dried formulation, to which the physician or the patient adds solvents and/or diluents prior to use.

In a further aspect, the pharmaceutical formulation comprises an aqueous solution of a FVII(a) polypeptide a bispecific antibody as described herein and a buffer, wherein the antibody is present in a concentration from 1 mg/ml or above and wherein said formulation has a pH from about 2.0 to about 10.0.

A composition of the invention may be administered parenterally, such as intravenously, such as intramuscularly, such as subcutaneously; preferably subcutaneously. The composition of the invention may be administered prophylactically.

A pharmaceutical composition of the invention may be used to treat a subject with a coagulopathy. As used herein, the term “subject” includes any human patient, or non-human vertebrate, with a coagulopathy.

### Medical Uses

The term “treatment”, as used herein, refers to the medical therapy of any human or other animal subject in need thereof. Said subject is expected to have undergone physical examination by a medical practitioner, who has given a tentative or definitive diagnosis which would indicate that the use of said specific treatment is beneficial to the health of said human or other animal subject. The timing and purpose of said treatment may vary from one individual to another, according to the status quo of the subject's health. Preventative or prophylactic administration of pro-coagulant compounds of the invention is also contemplated, with prevention being defined as delaying or averting manifestation or aggravation of one or more symptoms of the disease or disorder. Thus, said treatment may be prophylactic, palliative, symptomatic (“on demand”) and/or curative.

In terms of the present invention, prophylactic, palliative and/or symptomatic treatments may represent separate aspects of the invention.

A coagulopathy that results in an increased haemorrhagic tendency may be caused by any qualitative or quantitative deficiency of any pro-coagulant component of the normal coagulation cascade or any up-regulation of fibrinolysis. Such coagulopathies may be congenital and/or acquired and/or iatrogenic and are identified by a person skilled in the art.

Non-limiting examples of congenital hypocoagulopathies are haemophilia A, haemophilia B, Factor VII deficiency, Factor X deficiency, Factor XI deficiency, von Willebrand's disease and thrombocytopenias such as Glanzmann's thombasthenia and Bernard-Soulier syndrome.

A non-limiting example of an acquired coagulopathy is serine protease deficiency caused by vitamin K deficiency; such vitamin K-deficiency may be caused by administration of a vitamin K antagonist, such as warfarin. Acquired coagulopathy may also occur following extensive trauma. In this case otherwise known as the “bloody vicious cycle”, it is characterised by haemodilution (dilutional thrombocytopaenia and dilution of clotting factors), hypothermia, consumption of clotting factors and metabolic derangements (acidosis). Fluid therapy and increased fibrinolysis may exacerbate this situation. Said haemorrhage may be from any part of the body.

Haemophilia A with “inhibitors” (that is, allo-antibodies against factor VIII) and haemophilia B with “inhibitors” (that is, allo-antibodies against factor IX) are non-limiting examples of coagulopathies that are partly congenital and partly acquired.

A non-limiting example of an iatrogenic coagulopathy is that which is induced by excessive and/or inappropriate fluid therapy, such as that which may be induced by a blood transfusion.

In one preferred embodiment of the current invention, haemorrhage is associated with haemophilia A. In another preferred embodiment of the current invention, haemorrhage is associated with haemophilia B. In another preferred embodiment, haemorrhage is associated with haemophilia A or B with acquired inhibitors. In another preferred embodiment, haemorrhage is associated with FVII deficiency. In another preferred embodiment, haemorrhage is associated with Glansmann's thrombasthenia. In another embodiment, haemorrhage is associated with von Willebrand's disease. In another embodiment, haemorrhage is associated with severe tissue damage. In another embodiment, haemorrhage is associated with severe trauma. In another embodiment, haemorrhage is associated with surgery. In another embodiment, haemorrhage is associated with haemorrhagic gastritis and/or enteritis. In another embodiment, the haemorrhage is profuse uterine bleeding, such as in placental abruption. In another embodiment, haemorrhage occurs in organs with a limited possibility for mechanical haemostasis, such as intracranially, intra-aurally or intraocularly. In another embodiment, haemorrhage is associated with anticoagulant therapy.

In a further embodiment, haemorrhage may be associated with thrombocytopaenia. In individuals with thrombocytopaenia, the bispecific antibodies described herein may be co-administered with platelets.

### Routes of Administration

The bispecific antibodies described herein may be suitable for parenteral administration, preferably intravenous and/or subcutaneous administration. Subcutaneous administration is the preferred route of administration

### Dosing Regimens

The bioavailability and half-life of the bispecific antibodies described herein renders them particularly attractive medicaments for the prophylactic, subcutaneous treatment of subjects that have an acquired and/or congenital coagulopathy. The bispecific antibodies described herein may be administered once weekly, such as once every two weeks, preferably once monthly, to subjects that have an acquired and/or congenital coagulopathy but that are not bleeding. The bispecific antibodies described herein may be administered pre-operatively to subjects that have a coagulopathy and that are due for an invasive procedure, such as surgery. The bispecific antibodies described herein may be administered to subjects that have a coagulopathy and that are undergoing an invasive procedure, such as surgery.

The bispecific antibodies described herein may also be co-administered with exogenous FVIIa, such as plasma-derived or recombinantly produced FVIIa, for on-demand or prophylactic treatment of subjects that have a coagulopathy or are experiencing a bleeding episode.

The dose administered to subjects that have a coagulopathy will depend upon the route of administration, whether it is administered prophylactically or on demand and individual variation. Subcutaneous administration will necessitate larger doses than intravenous administration.

In an embodiment of the invention, the bispecific antibody is administered subcutaneously, in a dose from 1.0 to 30.0 nmol/kg.

Unless otherwise indicated in the specification, terms presented in singular form also include the plural situation.

### LIST OF EMBODIMENTS

The invention is further described by the following non-limiting list of embodiments of the present invention:
1. A bispecific antibody comprising
   (i) a first antigen binding site capable of binding Factor VII(a), and
   (ii) a second antigen binding site capable of binding TREM-like Transcript 1 (TLT-1).
2. The bispecific antibody according to embodiment 1, wherein the first antigen-binding site competes for binding to FVII(a) with any one of the anti-FVII(a) antibodies of Table 3:

| | | VL | VH | Constant |
|---|---|---|---|---|
| Antibody | Target | SEQ ID NO | SEQ ID NO | domain |
| mAb0522 | FVII(a) | 846 | 850 | B |
| Fab0883 | FVII(a) | 814 | 818 | G |
| mAb0005 | FVII(a) | 750 | 754 | M |
| mAb0004 | FVII(a) | 14 | 18 | M |
| mAb0013 | FVII(a) | 46 | 50 | M |
| mAb0018 | FVII(a) | 62 | 66 | M |
| mAb0544 | FVII(a) | 694 | 698 | M |
| mAb0552 | FVII(a) | 702 | 706 | M |
| mAb0001 | FVII(a) | 710 | 714 | M |
| mAb0007 | FVII(a) | 718 | 722 | M |
| mAb0578 | FVII(a) | 726 | 730 | M |
| mAb0701 | FVII(a) | 734 | 738 | M |
| mAb0587 | FVII(a) | 742 | 746 | M |

3. The bispecific antibody according to embodiment 1, wherein the antibody completes in a competitive ELISA assay as described in Example 7.

4. The bispecific antibody according to embodiment 1, wherein the first antigen-binding site is capable of binding an epitope comprising amino acid residues H115, T130, V131, and R392 of FVII(a) (SEQ ID NO: 1).
5. The bispecific antibody according to embodiment 1, wherein the first antigen-binding site is capable of binding an epitope comprising one or more of amino acid residues H115, T130, V131, and R392 of FVII(a) (SEQ ID NO: 1).
6. The bispecific antibody according to embodiment 1, wherein the first antigen-binding site is capable of binding an epitope comprising the following amino acid residues R113, C114, H115, E116, G117, Y118, S119, L120, T130, V131, N184, T185, P251, V252, V253, Q388, M391 and R392 of FVII(a) (SEQ ID NO: 1).
7. The bispecific antibody according to embodiment 1, wherein the first antigen-binding site is capable of binding an epitope comprising one or more of the following amino acid residues R113, C114, H115, E116, G117, Y118, S119, L120, T130, V131, N184, T185, P251, V252, V253, Q388, M391 and R392 of FVII(a) (SEQ ID NO: 1).
8. The bispecific antibody according to embodiment 1, wherein the first antigen-binding site comprises:
   the CDRL1 represented by SEQ ID NO: 847, with 0, 1, 2 or 3 amino acid substitutions
   the CDRL2 represented by SEQ ID NO: 848, with 0, 1, 2 or 3 amino acid substitutions
   the CDRL3 represented by SEQ ID NO: 849, with 0, 1 or 2 amino acid substitutions
   the CDRH1 represented by SEQ ID NO: 851, with 0 or 1 amino acid substitutions
   the CDRH2 represented by SEQ ID NO: 852, with 0 or 1 amino acid substitutions
   the CDRH3 represented by SEQ ID NO: 853, with 0, 1, 2 and 3 amino acid substitutions
9. The bispecific antibody according to embodiment 1, wherein the first antigen-binding site comprises:
   the CDRL1 represented by SEQ ID NO: 847,
   the CDRL2 represented by SEQ ID NO: 848,
   the CDRL3 represented by SEQ ID NO: 849,
   the CDRH1 represented by SEQ ID NO: 851,
   the CDRH2 represented by SEQ ID NO: 852, and
   the CDRH3 represented by SEQ ID NO: 853.
10. The bispecific antibody according to embodiment 1, wherein the first antigen-binding site comprises variable heavy chain and variable light chain domain sequences according to an antibody listed in Table 13 or 14, where the affinity (K_{D}) is 1 nM or less.
11. The bispecific antibody according to embodiment 1, wherein the first antigen-binding site comprises variable heavy chain and variable light chain domain sequences according to an antibody listed in Table 13 or 14, where the affinity (K_{D}) is 5 nM or less.
12. The bispecific antibody according to embodiment 1, wherein the first antigen-binding site comprises variable heavy chain and variable light chain domain sequences according to an antibody listed in Table 13 or 14, where the affinity (K_{D}) is 10 nM or less.
13. The bispecific antibody according to embodiment 1, wherein the first antigen-binding site comprises variable heavy chain and variable light chain domain sequences according to an antibody listed in Table 13 or 14, where the affinity (K_{D}) is 25 nM or less.
14. The bispecific antibody according to any one of the preceding embodiments, wherein the antibody have an affinity (K_{D}) for FVII(a) of less than 1 pM, such as less than 10 pM, such as less than 100 pM, such as less than 200 pM, such as less than 400 pM, such as less than 600 pM, such as less than 1 nM, such as less than 5 nM, such as less than 10 nM, such as less than 20 nM, such as less than 50 nM, such as less than 100 nM, such as less than 200 nM, such as less than 400 nM, such as less than 600 nM, such as less than 800 nM.
15. The bispecific antibody according to embodiment 1, wherein the first antigen-binding site comprises a light chain variable domain identified by SEQ ID NO:846 and a heavy chain variable domain identified by SEQ ID NO:850.
16. The bispecific antibody according to embodiment 15, wherein the light chain variable domain and/or the heavy chain variable domain sequences have 1, 2, 3, 4, 5, 6, 7, 8, 9, 10, 11 or 12 amino acid substitutions.
17. The bispecific antibody according to embodiment 1, wherein the second antigen-binding site competes for binding to TLT-1 with any one of the anti-TLT-1 antibodies of Table 4:

| | | VL | VH | Constant |
|---|---|---|---|---|
| Antibody | Target | SEQ ID NO | SEQ ID NO | domain |
| mAb0524 | TLT-1 | 854 | 858 | H |
| mAb0012 | TLT-1 | 862 | 866 | A |
| mAb0023 | TLT-1 | 870 | 874 | A |
| mAb0051 | TLT-1 | 878 | 882 | A |
| mAb0062 | TLT-1 | 894 | 898 | A |

18. The bispecific antibody according to embodiment 17, wherein the antibody competes in a competitive ELISA assay as described in Example 32.
19. The bispecific antibody according to embodiment 1, wherein the second antigen-binding site is capable of binding an epitope comprising the following amino acid residues K8, I9, G10, S11, L12, A13, N15, A16, F17, S18, D19, P20, A21 of TLT-1 (SEQ ID NO: 13).
20. The bispecific antibody according to embodiment 1, wherein the second antigen-binding site comprises:
   the CDRL1 represented by SEQ ID NO: 855, with 0, 1, 2 or 3 amino acid substitutions
   the CDRL2 represented by SEQ ID NO: 856, with 0, 1 or 2 amino acid substitutions
   the CDRL3 represented by SEQ ID NO: 857, with 0, 1 or 2 amino acid substitutions
   the CDRH1 represented by SEQ ID NO: 859, with 0 or 1 amino acid substitutions
   the CDRH2 represented by SEQ ID NO: 860, with 0, 1, 2 or 3 amino acid substitutions
   the CDRH3 represented by SEQ ID NO: 861. with 0 or 1 amino acid substitutions
21. The bispecific antibody according to embodiment 1, wherein the second antigen-binding site comprises:
   the CDRL1 represented by SEQ ID NO: 855,
   the CDRL2 represented by SEQ ID NO: 856,
   the CDRL3 represented by SEQ ID NO: 857,
   the CDRH1 represented by SEQ ID NO: 859,
   the CDRH2 represented by SEQ ID NO: 860,
   the CDRH3 represented by SEQ ID NO: 861.
22. The bispecific antibody according to embodiment 1, wherein the second antigen-binding site comprises:
   the CDRL1 represented by SEQ ID NO: 871,
   the CDRL2 represented by SEQ ID NO: 872,
   the CDRL3 represented by SEQ ID NO: 873,
   the CDRH1 represented by SEQ ID NO: 875,
   the CDRH2 represented by SEQ ID NO: 876, and
   the CDRH3 represented by SEQ ID NO: 877.
23. The bispecific antibody according to embodiment 1, wherein the second antigen-binding site comprises:
   the CDRL1 represented by SEQ ID NO: 879,
   the CDRL2 represented by SEQ ID NO: 880,
   the CDRL3 represented by SEQ ID NO: 881,
   the CDRH1 represented by SEQ ID NO: 883,
   the CDRH2 represented by SEQ ID NO: 884, and
   the CDRH3 represented by SEQ ID NO: 885.
24. The bispecific antibody according to embodiment 1, wherein the second antigen-binding site comprises:
   the CDRL1 represented by SEQ ID NO: 895,
   the CDRL2 represented by SEQ ID NO: 896,
   the CDRL3 represented by SEQ ID NO: 897,
   the CDRH1 represented by SEQ ID NO: 899,
   the CDRH2 represented by SEQ ID NO: 900, and
   the CDRH3 represented by SEQ ID NO: 901.
25. The bispecific antibody according to embodiment 1, wherein the second antigen-binding site is comprised by a light chain variable domain identified by SEQ ID NO:854 and a heavy chain variable domain identified by SEQ ID NO:858.
26. The bispecific antibody according to embodiment 25, wherein the light chain variable domain and/or the heavy chain variable domain sequences have 1, 2, 3, 4, 5, 6, 7, 8, 9, 10, 11 or 12 amino acid substitutions.
27. The bispecific antibody according to any one of the preceding embodiments, wherein the antibody have an affinity (K_{D}) for TLT-1 of less than 1 pM, such as less than 10 pM, such as less than 100 pM, such as less than 200 pM, such as less than 400 pM, such as less than 600 pM, such as less than 1 nM, such as less than 5 nM, such as less than 10 nM, such as less than 20 nM, such as less than 50 nM, such as less than 100 nM, such as less than 200 nM, such as less than 400 nM, such as less than 600 nM, such as less than 800 nM.
28. The bispecific antibody according to embodiment 1, wherein the first antigen-binding site comprises a light chain variable domain identified by SEQ ID NO:846 and a heavy chain variable domain identified by SEQ ID NO:850, and wherein the second antigen-binding site is comprised by a light chain variable domain identified by SEQ ID NO: 854 and a heavy chain variable domain identified by SEQ ID NO:858.
29. The bispecific antibody according to any one of the preceding embodiments, wherein the antibody comprises an Fc region.
30. The bispecific antibody according to embodiment 29, wherein the Fc region is an Fc variant of the Fc region of IgG1 comprising the substitutions L234A, L235E, G237A, A330S and P331S.
31. The bispecific antibody according to any one of the preceding embodiments, wherein the antibody is a fusion or conjugate of antigen binding fragments of antibodies.
32. The bispecific antibody according to embodiment 31, wherein one or more of the binding fragments are selected from the group of a Fab, a Fab′, a Fab2, a Fab′2 and a scFv.
33. The bispecific antibody according to any one of the preceding embodiments, wherein the antibody is a multispecific antibody.
34. The bispecific antibody according to embodiment 1, wherein the first antigen-binding site comprises a first light chain variable domain identified by SEQ ID NO:846 and a first heavy chain variable domain identified by SEQ ID NO:850, and wherein the second antigen-binding site is comprised by a second light chain variable domain identified by SEQ ID NO: 854 and a second heavy chain variable domain identified by SEQ ID NO:858 and wherein the heavy chain constant domains attached to first and second heavy chain variable domain are identified by SEQ ID NO: 5 and 4, respectively and wherein the light chain constant domains attached to first and second light chain variable domain are both identified by SEQ ID No: 12.
35. The bispecific antibody according to embodiment 1, wherein the first antigen-binding site comprises a first light chain variable domain identified by SEQ ID NO:846 and a first heavy chain variable domain identified by SEQ ID NO:850, and wherein the second antigen-binding site is comprised by a second light chain variable domain identified by SEQ ID NO: 854 and a second heavy chain variable domain identified by SEQ ID NO:858 and wherein the heavy chain constant domains attached to first and second heavy chain variable domain are identified by SEQ ID NO: 943 and 942, respectively and wherein the light chain constant domains attached to first and second light chain variable domain are both identified by SEQ ID No: 12
36. The bispecific antibody according to embodiment 1, wherein the first antigen-binding site comprises a first light chain variable domain identified by SEQ ID NO:846 and a first heavy chain variable domain identified by SEQ ID NO:850, and wherein the second antigen-binding site is comprised by a second light chain variable domain identified by SEQ ID NO: 854 and a second heavy chain variable domain identified by SEQ ID NO:858 and wherein the heavy chain constant domains attached to first and second heavy chain variable domain are identified by SEQ ID NO: 7 and 6, respectively and wherein the light chain constant domains attached to first and second light chain variable domain are both identified by SEQ ID No: 12.
37. The bispecific antibody according to embodiment 1, wherein the first antigen-binding site comprises a first light chain variable domain identified by SEQ ID NO:846 and a first heavy chain variable domain identified by SEQ ID NO:850, and wherein the second antigen-binding site is comprised by a second light chain variable domain identified by SEQ ID NO: 854 and a second heavy chain variable domain identified by SEQ ID NO:858 and wherein the heavy chain constant domains attached to first and second heavy chain variable domain are identified by SEQ ID NO: 4 and 5, respectively and wherein the light chain constant domains attached to first and second light chain variable domain are both identified by SEQ ID NO: 12.
38. The bispecific antibody according to embodiment 1, wherein the first antigen-binding site comprises a first light chain variable domain identified by SEQ ID NO:846 and a first heavy chain variable domain identified by SEQ ID NO:850, and wherein the second antigen-binding site is comprised by a second light chain variable domain identified by SEQ ID NO: 854 and a second heavy chain variable domain identified by SEQ ID NO:858 and wherein the heavy chain constant domains attached to first and second heavy chain variable domain are identified by SEQ ID NO: 942 and 943, respectively and wherein the light chain constant domains attached to first and second light chain variable domain are both identified by SEQ ID NO: 12.
39. The bispecific antibody according to embodiment 1, wherein the first antigen-binding site comprises a first light chain variable domain identified by SEQ ID NO:846 and a first heavy chain variable domain identified by SEQ ID NO:850, and wherein the second antigen-binding site is comprised by a second light chain variable domain identified by SEQ ID NO: 854 and a second heavy chain variable domain identified by SEQ ID NO:858 and wherein the heavy chain constant domains attached to first and second heavy chain variable domain are identified by SEQ ID NO: 6 and 7, respectively and wherein the light chain constant domains attached to first and second light chain variable domain are both identified by SEQ ID NO: 12.
40. The bispecific antibody according to embodiment 1, wherein the antibody competes for binding to FVII(a) and competes for binding to TLT-1 with one or more of the bispecific antibodies as set forth in Table 5 (where constant domain abbreviation is defined according to table 2a):

TABLE 5

| biAb | anti-FVII(a) mAb | anti-TLT-1 mAb | Constant domain |
|---|---|---|---|
| biAb0001 | mAb0865 | mAb1076 | H |
| biAb0352 | mAb0350 | mAb0351 | I |
| biAb0015 | mAb0865 | mAb1038 | H |
| biAb0090 | mAb0865 | mAb1049 | H |
| biAb0095 | mAb0865 | mAb1047 | H |
| biAb0100 | mAb0522 | mAb0524 | L |
| biAb0241 | mAb0865 | mAb0023 | H |
| biAb0242 | mAb0865 | mAb0051 | H |
| biAb0243 | mAb0865 | mAb0062 | H |
| biAb0244 | mAb0864 | mAb1076 | H |
| biAb0245 | mAb0873 | mAb1076 | H |
| biAb0011 | mAb0875 | mAb1076 | H |
| biAb0016 | mAb0875 | mAb1038 | H |
| biAb0096 | mAb0875 | mAb1047 | H |
| biAb0091 | mAb0875 | mAb1049 | H |
| biAb0012 | mAb0872 | mAb1076 | H |
| biAb0017 | mAb0872 | mAb1038 | H |
| biAb0097 | mAb0872 | mAb1047 | H |
| biAb0092 | mAb0872 | mAb1049 | H |
| biAb0013 | mAb0874 | mAb1076 | H |
| biAb0018 | mAb0874 | mAb1038 | H |
| biAb0098 | mAb0874 | mAb1047 | H |
| biAb0093 | mAb0874 | mAb1049 | H |
| biAb0014 | mAb0873 | mAb1076 | H |
| biAb0019 | mAb0873 | mAb1047 | H |
| biAb0099 | mAb0873 | mAb1047 | H |
| biAb0094 | mAb0873 | mAb1049 | H |

41. The bispecific antibody according to embodiment 38, wherein the antibody competes for binding in a competitive ELISA to FVII(a) and competes for binding to TLT-1 with one or more of the bispecific antibodies as set forth in Table 5.
42. The bispecific antibody according to embodiment 38, wherein the antibody competes in a competitive ELISA assay as described in Example 7 and Example 32.
43. The bispecific antibody according to embodiment 1, wherein the first antigen-binding site is capable of binding an epitope comprising amino acid residues H115, T130, V131, and R392 of FVII(a) (SEQ ID NO: 1), and wherein the second antigen-binding site is capable of binding an epitope comprising the following amino acid residues K8, I9, G10, S11, L12, A13, N15, A16, F17, S18, D19, P20, A21 of TLT-1 (SEQ ID NO: 13).
44. The bispecific antibody according to embodiment 1, wherein the first antigen-binding site comprises:
   the CDRL1 represented by amino acid residues (SEQ ID NO: 847), with 0, 1, 2 or 3 amino acid substitutions
   the CDRL2 represented by amino acid residues (SEQ ID NO: 848), with 0, 1, 2 or 3 amino acid substitutions
   the CDRL3 represented by amino acid residues (SEQ ID NO: 849), with 0, 1 or 2 amino acid substitutions
   the CDRH1 represented by amino acid residues (SEQ ID NO: 851),
   the CDRH2 represented by amino acid residues (SEQ ID NO: 852), with 0 or 1 amino acid substitutions
   the CDRH3 represented by amino acid residues (SEQ ID NO: 853), with 0, 1, 2 and 3 amino acid substitutions, and
      wherein the second antigen-binding site comprises:
   the CDRL1 represented by SEQ ID NO: 855, with 0, 1, 2 or 3 amino acid substitutions
   the CDRL2 represented by SEQ ID NO: 856, with 0, 1 or 2 amino acid substitutions
   the CDRL3 represented by SEQ ID NO: 857, with 0, 1 or 2 amino acid substitutions
   the CDRH1 represented by SEQ ID NO: 859, with 0 or 1 amino acid-substitutions
   the CDRH2 represented by SEQ ID NO: 860, with 0, 1, 2 or 3 amino a acid-substitutions
   the CDRH3 represented by SEQ ID NO: 861. with 0 or 1 amino acid substitutions.
45. The bispecific antibody according to any one of the preceding embodiments, wherein the antibody increases the functional MRT of FVII(a), as measured with a FVIIa activity assay described in Example 8.
46. The bispecific antibody according to embodiment 41, wherein the antibody increases the functional MRT of FVII(a) by at least 2-fold, at least 3-fold, at least 5-fold, at least 10-fold, at least 20-fold, at least 30-fold, or at least 40-fold compared with administration of FVII(a) in the absence of the bispecific antibody of the invention.
47. The bispecific antibody according to any one of the preceding embodiments, wherein the antibody is capable of increasing the level of circulating endogenous functionally active FVIIa as determined according to Examples 27 and 28, compared to the level of circulating endogenous FVIIa in the absence of administered bispecific antibody.
48. The bispecific antibody according to embodiment 45, wherein the antibody increases the level of circulating endogenous functionally active FVIIa by at least 2-fold, at least 4-fold, at least 10-fold, at least 20-fold, at least 40-fold, at least 80-fold, at least 160-fold, at least 320-fold, at least 640-fold.
49. The bispecific antibody according to any one of the preceding embodiments, wherein the antibody is capable of increasing the ability of a FVIIa polypeptide to promote FX activation as determined by stimulatory activity assay as described in Example 21.
50. The bispecific antibody according to embodiment 43, wherein the stimulatory activities are at least 2-fold, at least 5-fold, at least 10-fold, at least 20-fold, at least 30-fold, at least 40-fold, at least 60-fold, at least 80-fold, at least 100-fold, or at least 150-fold.
51. A pharmaceutical formulation comprising the bispecific antibody according to any one of the preceding embodiments and a pharmaceutically acceptable carrier.
52. The bispecific antibody according to embodiments 1-48, wherein the pharmaceutical formulation according to embodiment 49 is administered by subcutaneous administration.
53. The bispecific antibody according to embodiments 1-48 or the pharmaceutical formulation according to embodiments 49 for use as a medicament.
54. The bispecific antibody according to any one of embodiments 1-48, and 50-51 or the pharmaceutical formulation according to embodiments 49 for use in the treatment of a coagulopathy, wherein said coagulopathy is congenital and/or acquired.
55. The bispecific antibody according to embodiment 52 wherein said coagulopathy is selected from the group consisting of haemophilia A, with or without inhibitors or haemophilia B, with or without inhibitors, FVII(a) deficiency and Glanzmann's thrombasthenia.
56. The bispecific antibody according to embodiments 1-48, and 50-51 for treatment of a bleeding, wherein the bleeding is associated with congenial or acquired haemophilia A, congenial or acquired haemophilia B, haemophilia A with inhibitors, haemophilia B with inhibitors or Factor VII(a) deficiency.
57. The use according to any one of embodiments 1-48, wherein said bispecific antibody or pharmaceutical formulation according to embodiment 49 is parenterally administered, such as intravenously, intramuscularly or subcutaneously administered.

### EXAMPLES

### Example 1: Generation of Anti-FVII(a) Mouse Monoclonal Antibodies Using Hybridoma Technology

Monoclonal antibodies were prepared by immunisation of NMRCF1 mice (Charles River) with the FVIIa Q64C-sTF(1-219) G109C disulfide-linked complex described in the international patent application with publication number WO07/115953.

The mice were given an initial subcutaneous injection of 20 μg in FCA (Freund's complete adjuvant) followed by booster intraperitoneal injections in IFA (Freund's incomplete adjuvant) of 20 μg antigen. The spleens were removed aseptically and dispersed to a single cell suspension. Splenocytes were fused to X63Ag8.653 myeloma cells using electrofusion. Cells were seeded in microtiter plates and cultured at 37° C., 5% CO2. The tissue culture medium (RPMI 1640+10% fetal calf serum), containing HAT (Sigma) for selection, was changed twice. Following 10 days growth, specific antibody-producing hybridoma clones were identified by ELISA screening using the following protocol. NUNC Maxisorb plates were coated with FVIIa Q64C-sTF(1-219) G109C disulfide-linked complex, or FVIIa (expressed and purified as described by Thim et al. (1988) Biochemistry 27:7785-7793 and Persson et al. (1996) FEBS Lett 385:241-243), 50 μl/well at 1 μg/ml (HEPES buffer containing 5 mM CaCl2), and incubated overnight at 4° C. The plates were washed 5 times and blocked in washing buffer for 15 min (HEPES buffer, 5 mM CaCl2, 0.05% TWEEN 20). 50 μl supernatant was transferred to each well and incubated for 1 hour. The plates were washed 5 times and 50 μl HRP-labelled goat-anti mouse was added (Fc gamma fragment specific; Jackson, working dilution 1/10000). The plates were incubated for 1 hour, washed 5 times, and developed with 50 μl TMB (TMB ONE ready to use; Kem-En-Tec) for 10 min. The reaction was stopped by adding 50 μl 4M H3PO4 and read in a FLUOStar Optima plate reader at 450 and 620 nm.

Hybridoma cells yielding positive results were subcloned at least twice by limiting dilution in order to secure monoclonality. Antibody purification was done using standard protein A purification.

Hybridoma cells producing antibodies for use in PK studies were up-scaled into T-flasks or shaker flasks, in RPMI1640+10% FBS medium. Conditioned medium was harvested by centrifugation, and antibodies purified by protein A affinity chromatography followed by desalting.

### Example 2: Cloning and Sequencing of Mouse Anti-FVII(a) Antibodies

This example describes cloning and sequencing of the murine heavy chain (HC) and light chain (LC) cDNA sequences encoding the variable domains of an anti-FVII(a) antibody. Total RNA was extracted from hybridoma cells using the RNeasy-Mini Kit from Qiagen and used as template for cDNA synthesis. cDNA was synthesized in a 5′-RACE reaction using the SMARTer RACE cDNA amplification kit from Clontech. Subsequent target amplification of the variable domain of the HC (designated VH) and of the variable domain of the LC (designated VL) sequences was performed by PCR using Phusion Hot Start polymerase (Finnzymes) and the universal primer mix (UPM) included in the SMARTer™ RACE kit as forward primer. The sequence of the reverse primer used for VH amplification was 5′ agctgggaaggtgtgcacac 3′ (SEQ ID NO: 944). The sequence of the reverse primer used for VL amplification was 5′ gctctagactaacactcattcctgttgaagctcttg 3′ (SEQ ID NO:945).

PCR products were separated by gel electrophoresis, extracted using the GFX PCR DNA & Gel Band Purification Kit from GE Healthcare Bio-Sciences and cloned for sequencing using a Zero Blunt TOPO PCR Cloning Kit and chemically competent TOP10 E. coli (Invitrogen). Sequencing was performed at MWG Biotech, Martinsried Germany using M13uni(-21)/M13rev(-29) sequencing primers. All kits and reagents were used according to the manufacturer's instructions.

### Example 3: Recombinant Expression of Bivalent Antibodies, Monovalent Antibodies (OA Antibodies), and Antibody Fab Fragments

Bivalent antibodies, monovalent antibodies (one-armed referred to as OA-antibodies) and antibody Fab fragments were expressed using transient transfection of HEK293 suspension cells (293Expi, Invitrogen) essentially following the manufacturer's instructions. 293Expi cells were sub-cultivated every 3-4 days in Expi293F expression medium (Invitrogen, catalogue number A1435104) supplemented with 1% P/S (GIBCO catalogue number 15140-122). Expi293F cells were transfected at a cell density of 2.5-3 million/ml using Expifectamine. For each litre of Expi293F cells, the transfection was performed by diluting a total of 1 mg of plasmid DNA into 50 ml Optimem (GIBCO, cat. no. 51985-026, dilution A) and by diluting 2.7 ml Expifectamine into 50 ml Optimem (dilution B). Bivalent antibodies were produced by co-transfecting VH-CH1-CH2-CH3 (HC) and VL-CL (LC) plasmid (1:1 ratio), and for Fab fragments plasmids were VH-CH1 and LC (1:1 ratio). For production of OA antibodies, the cells were transfected with three plasmids: the HC the LC plasmids, and a third plasmid encoding a truncated HC (trHC). The HC of the OA-antibodies contained the hole mutations (T366S, L368A, Y407V) and the trHC the knob (T366W) mutation, but knobs and holes can also be reversed. The knob/hole mutations are described in the international patent EP0979281B1 and are introduced to optimize formation of the desired hetero-dimer, i.e. pairing of HC and trHC, and suppress unwanted formation of homodimers i.e. pairing of trHC with trHC and HC with HC. Dilution A and B were mixed and incubated at room temperature for 10-20 minutes. The transfection mix was hereafter added to the Expi293F cells and cells were incubated at 37° C. in a humidified incubator with orbital rotation (85-125 rpm). One-day post-transfection, transfected cells were supplemented with 5 ml of ExpiFectamine 293 Transfection Enhancer 1 and 50 ml of ExpiFectamine 293 Transfection Enhancer 2. Cell culture supernatants were harvested 4-5 days post-transfection by centrifugation followed by filtration.

Bivalent and monovalent antibodies were purified by standard protein A affinity chromatography known to a person skilled in the art and, where necessary, additional purification steps such as gel filtration or ion exchange chromatography. Fab fragments were purified by affinity chromatography using an affinity resin recognizing the kappa chain of the Fab.

### Example 4: Preparation of Bispecific Antibodies and OA Antibodies by In Vitro Assembly

### Bispecific Antibodies Prepared by In Vitro Assembly

Bispecific antibodies were generated by in vitro assembly of two parental antibodies by a method similar to that described in Labrijn et al. PNAS 2013, vol. 110, pp. 5145-5150, and known as the DuoBody® technology (Genmab). Instead of the IgG1 subtype used by Labrijn et al. IgG4 was used in the present invention. The exchange reaction was carried out at 30° C. for four hours in the presence of 75 mM 2-mercaptoethyl amine (2-MEA). The resulting bispecific antibody was purified by ion exchange chromatography to separate residual parental antibodies from the bispecific antibody. In the present example, the heavy chain constant region of the first parental antibody (anti-FVII(a)) was IgG4 S228P F405L R409K and the heavy chain constant region of the second parental antibody (anti-TLT-1) was IgG4 S228P (both using EU numbering). The light chain constant domains were human kappa. The two parental antibodies were produced as described in example 3. The bispecific anti-FVII(a)/anti-TLT-1 antibodies could also be assembled from a set of parental antibodies where the constant domain of the anti-FVII(a) antibody is IgG4 S228P and the constant domain of the anti-TLT-1 antibody is IgG4 S228P F405L R409K.

### Monovalent Antibodies Prepared by In Vitro Assembly

Monovalent antibodies were generated by in vitro assembly as described above for bispecific antibodies with the exceptions that (1) instead of combining two antibodies to form a bispecific antibody, an antibody and a trHC dimer were combined to form the monovalent antibody. In the present example the trHC was IgG4 S228P F405L R409K and the HC was IgG4 S228P (both using EU-numbering). The light chain constant domain was human kappa. Typically arm-exchange reaction was carried out with the trHC dimer in 20-50% molar excess to minimize the amount of bivalent antibody in the reaction mixture. The monovalent antibody was purified by size-exclusion chromatography, optionally supplemented by additional purification steps, such as ion exchange chromatography, as desired.

### Example 5: In Vitro Characterization of Anti-FVIIa(a) in Functional Assays

In order to facilitate accumulation of endogenous FVIIa and allow it to exert its procoagulaint activity, anti-FVII(a) antibodies of the present invention should preferably not impair the activity of FVIIa and similarly preferably not promote the inactivation of FVIIa by its primary plasma inhibitor antithrombin (Agersø H, et al. (2011) J Thromb Haemost 9:333-338). To explore these aspects, the effect of anti-FVII(a) antibodies on the procoagulant activity of FVIIa and inactivation of FVIIa by antithrombin was determined in vitro using the assays described below.

### Effect of Anti-FVII(a) Antibodies on Thrombin Generation in Hemophilia A-Induced Human Plasma

The effect of bivalent or monovalent anti-FVII(a) antibodies on thrombin generation was measured in a kaolin-triggered thrombin generation assay (TGT) in a 96-well setup. In brief, hemophilia A-induced plasma was prepared by addition of an anti-hFVIII antibody 4F30 (described in the international patent application with publication number WO2012/035050) to a final conc. of 37.5 μg/ml. Phospholipids at a final concentration of 10 μM (Rossix) was added to the hemophilia A plasma and incubated at 37° C. for 15 min. Purified antibody (100 nM) and FVIIa (25 nM) were added to the mixture and incubated for 10 min at room temperature. The anti-FVII(a) antibodies were generated as described in examples 1, 2, and 3. Triggering was done by adding 10 μl Kaolin (Haemonetics) followed by addition of 10 μl FIIa FluCa-kit (Thrombinoscope BV) and fluorescence (excitation at 390 nm and emission at 460 nm) was measured on an EnVision multi-label reader for two hours.

Thrombograms were calculated as the first derivative of the measured fluorescence. Peak thrombin height was calculated as the maximum value in the thrombogram. This was subsequently normalized and expressed as percentage by dividing the observed peak height with the peak height corresponding to that observed for 25 nM FVIIa in the absence of antibody. Based on this, antibodies were categorized as stimulatory (>120%), inhibitory (<90%) or neutral (90%- 120%). As shown in Table 6, antibodies from all categories were identified. Preferred antibodies were stimulatory or neutral in the TGT assay.

### Effect of Anti-FVII(a) Antibodies on FVIIa Inactivation by Plasma Derived Antithrombin

Inhibition of FVIIa activity by plasma-derived antithrombin (AT) in the presence of bivalent or monovalent anti-FVII(a) antibody was measured by incubating FVIIa (200 nM) with low-molecular weight heparin (Enoxaparin, 12 μM) and the antibody (200-1000 nM) for 10 min in 50 mM HEPES, 100 mM NaCl, 10 mM CaCl2, 0.1% PEG8000, 1 mg/ml BSA, pH 7.3. AT (5 μM) was then added and at time points 10, 20, 30, 40, 60, and 80 min a sample was transferred to a new microtiter plate, where residual activity was measured in the presence of 1 mM S-2288 chromogenic substrate (Chromogenix), 200 nM soluble tissue factor (sTF; produced as described by Freskgard et al. (1996) Protein Sci 5:1531-1540) and polybrene (0.5 mg/ml) in a Spectramax instrument (Molecular Devices) at 405 nm for 5 min. Sample with buffer instead of AT provided the activity of uninhibited FVIIa.

Residual amidolytic activity was determined as a function of time relative to the activity in the absence of inhibitor. The inhibitory constant (kinh) was estimated by fitting the data to a one phase decay model. The value of kinh in the presence of FVII(a)-antibody in percent of that determined in the absence of antibody is termed kinh % and is reported for each antibody in Table 6.

Antibodies with an estimated kinh %<60% were classified as protecting FVIIa from AT inhibition. Antibodies with an estimated 60%≤kinh %≤150% were classified as neutral, while antibodies with an estimated kinh %>150% were classified as accelerating the inhibition of FVIIa by AT. As reported in Table 6, antibodies from all three categories were identified. Preferred antibodies were neutral or protected FVIIa from AT inhibition.

Among the tested antibodies, a subset of antibodies including 11F2 (mAb005 and mAb0048, corresponding to fully murine and murine/human chimera, respectively) were found to have desired in vitro properties as described above.

TABLE 6
Functional characterization of anti-FVII(a) antibodies
in thrombin generation (TGT) and antithrombin
(AT)-inhibition assays as described in Example 2.

| | | Ranking | | Ranking |
|---|---|---|---|---|
| Anti- | TGT | based | AT | based |
| FVII(a) | activity | on TGT | inhibition | on AT |
| antibody | (% FVIIa) | activity | (k_{inh %}) | inhibition |
| mAb0048 | n.d. | Neutral | 109 | Neutral |
| mAb0005 | 115 | | 133 | |
| mAb0007 | 113 | | 121 | |
| mAb0544 | 103 | | 111 | |
| mAb0552 | 104 | | 118 | |
| mAb0018 | 141 | Stimulatory | 525 | Accelerating |
| mAb0004 | 75 | Inhibitory | 24 | Protective |
| mAb0001 | 109 | Neutral | 82 | Neutral |
| mAb0578 | 91 | Neutral | 77 | Neutral |
| mAb0587 | 93 | Neutral | 103 | Neutral |
| mAb0013 | 128 | Stimulatory | 747 | Accelerating |
| mAb7250 | 91 | Neutral | 23 | Protective |
| mAb0701 | 88 | Inhibitory | 26 | Protective |

### Example 6: SPR Analysis of Antibody Binding to FVIIa and the Effect of pH and Calcium

Binding of antibodies from Example 5 to FVIIa was probed by surface plasmon resonance (Biacore T200). Anti-murine IgG (by GE Healthcare) was immobilised on a CM4 sensor chip using standard amine coupling chemistry kit (both supplied by GE Healthcare). Purified anti-FVII(a) antibodies according to Table 7 (0.25 nM) were injected at a flow rate of 10 μl/min for 1 min. Subsequently, 5, 15, 45 and 135 nM of FVIIa were injected at a flow rate of 30 μl/min for 7 min to allow for binding to the anti-FVII(a) antibody followed by a 9 min buffer injection allowing for dissociation from the anti-FVII(a) antibody. The running buffer was prepared by diluting 10-fold the 10×HBS-P buffer (supplied by GE Healthcare) and supplemented with 1 mg/ml BSA and 5 mM CaCl2 to give 10 mM HEPES, 150 mM NaCl, 0.05% v/v Polysorbate 20 (P20), pH 7.4, 5 mM CaCl2, 1 mg/ml bovine serum albumin (BSA). The running buffer was also used for dilution of anti-FVII(a) antibody and FVII samples. Regeneration of the chip was achieved using regeneration buffer consisting of 10 mM Gly-HCl pH 1.7 (supplied by GE Healthcare). Binding data were analysed according to a 1:1 model using BiaEvaluation 4.1 supplied by the manufacturer (Biacore AB, Uppsala, Sweden). Analysis resulted in the binding constants reported in Table 7 demonstrating high-affinity binding of several antibodies to FVIIa including 11F2 (mAb005 and mAb0048, corresponding to fully murine and murine/human chimera, respectively).

TABLE 7
Estimated binding constants for the interaction of
anti-FVII(a) antibodies with FVIIa as determined by
surface plasmon resonance (SPR) analysis according to Example 6.

| Anti-FVII(a) antibody | K_{D} (M) |
|---|---|
| mAb0001 | 1.7E−10 |
| mAb0007 | 2.5E−10 |
| mAb0544 | 2.7E−10 |
| mAb0552 | 7.6E−10 |
| mAb0578 | 5.8E−10 |
| mAb0587 | 4.9E−10 |
| mAb0005 | 1.4E−10 |
| mAb0004 | 1.6E−10 |
| mAb0018 | 9.3E−9 |
| mAb0048 | 2.9E−11 |

The effect of pH and CaCl2 on the binding of select antibodies from Example 5 to FVIIa and cFVIIa-chimera was probed by surface plasmon resonance (Biacore T200) at 37° C. cFVIIa-chimera sequence is indicated in the section titled detailed description of the invention and is expressed as outlined in examples 16 and 26. Anti-murine IgG (by GE Healthcare) was immobilised on a CM4 sensor chip using standard amine coupling chemistry kit (both supplied by GE Healthcare). A pre-equilibrated mixture of FVIIa at 1.2 nM and 4F9 (NN internal anti-FVII murine Ab binding to FVIIa EGF1 domain) at 0.5 nM was injected at a flow rate of 10 μl/min for 1 min. Subsequently, 540, 180, 60, 20, 6.66, 2.22, 0.74, 0.25 nM of anti-FVII(a) antibody were injected at a flow rate of 30 μl/min for 7 min to allow for binding to FVIIa followed by a 9 min buffer injection allowing for dissociation from FVIIa. Two running buffers were prepared. Buffer 1 was prepared by diluting 10-fold of the 10×HBS-P+ (supplied by GE Healthcare) and supplemented with 1 mg/ml BSA and 5 mM CaCl2 to give 10 mM HEPES, 150 mM NaCl, 0.05% v/v Polysorbate 20, pH 7.4, 5 mM CaCl2, 1 mg/ml bovine serum albumin (BSA). Buffer 2 was prepared by diluting 10-fold of the 10×HBS-P+ (supplied by GE Healthcare) and supplemented with 1 mg/ml BSA, 5 UM CaCl2, and adjusted pH to 6.0 (adjusted using 4M HCl) to give 10 mM HEPES, 150 mM NaCl, 0.05% v/v Polysorbate 20, pH 6.0, 5 μM CaCl2, 1 mg/ml bovine serum albumin (BSA). FVIIa, anti-FVII antibody 4F9, and anti-FVII(a) antibody were diluted in both the running buffers separately. Regeneration of the chip was achieved using regeneration buffer consisting of 10 mM Gly-HCl pH 1.7 (supplied by GE Healthcare). Binding data were analysed according to a 1:1 model using BiaEvaluation 4.1 supplied by the manufacturer (Biacore AB, Uppsala, Sweden). Analysis resulted in the binding constants reported in Table 7b demonstrating retained high-affinity binding between FVIIa and anti-FVII(a) antibody.

TABLE 7b
Estimated binding constants and fold-difference for the interaction of
FVIIa and anti-FVII(a) antibodies in two different buffers as determined
by surface plasmon resonance (SPR) analysis according to Example 6.

| | | K_{D,1} | K_{D,2} | Fold- |
|---|---|---|---|---|
| | | In Buffer 1, | In Buffer 2, | difference, |
| mAb | Protein | (M) | (M) | (K_{D,2}/K_{D,1}) |
| mAb0077(OA) | FVIIa | 304E−12 | 495E−12 | 1.6 |
| | cFVIIa- | 469E−12 | 652E−12 | 1.4 |
| | chimera | | | |
| mAb0842(OA) | FVIIa | 599E−12 | 2200E−12 | 3.7 |
| | cFVIIa- | 1020E−12 | 1600E−12 | 1.6 |
| | chimera | | | |

### Example 7: Identification of Antibodies Competing With mAb0005 (11F2) for Binding to FVII(a) in a Competitive ELISA

To determine if anti-FVII(a) antibodies with desired in vitro properties from Example 5 compete with mAb0005 (11F2), and antibodies derived thereof, for binding to FVIIa(a) competition studies were performed with the corresponding Fab fragment, Fab0076. FVIIa, H-D-Phe-Phe-Arg chloromethyl ketone (FFR-cmk; Bachem, Switzerland) active-site inhibited FVIIa (FVIIai; see Example 9) was immobilised on a NUNC maxisorp plate at a concentration of 125 ng/ml in dilution buffer (20 mM HEPES, 5 mM CaCl2, 150 mM NaCl, pH 7.2) over night at 4° C. The plates were washed and blocked in washing buffer (20 mM HEPES, 5 mM CaCl2, 150 mM NaCl, 0.5 mL/L Tween 20, pH 7.2) for 15 min. 11F2-Fab0076 was biotinylated using a standard biotinylation kit (EZ-Link, Thermo) used according to manufacturer's instructions. For the competition study, biotinylated Fab0076 at a final fixed concentration of 10 ng/ml was combined with a dilution series of anti-FVIIa(a) antibody to give final concentrations ranging from 100 mg/ml to 9.5 ng/ml in dilution buffer. The mixture was added to the wells of the plate and allowed to incubate for 1 hour. The plate was then washed and HRP-labelled streptavidin-HRPO (1:2000 in dilution buffer; Kirkegaard & Perry Labs) was added and incubated for 1h. Finally, the plate was washed and developed with TMB ONE (KEMENTEC) for 10 min. The reaction was stopped by adding H3PO4 (4M) and the plate read in a FLUOStar Optima plate reader at 450 nm with subtraction of the background signal measured at 620 nm. Unless otherwise specified, all incubations were done at room temperature and plates were washed 5 times using washing buffer.

From the measured signals (OD units), competition at any given antibody concentration was calculated as
% inhibition=(1−(OD units−100% inhibition)/(0% inhibition−100% inhibition))*100 where 0% inhibition was determined from the signal in wells without any competing anti-FVII(a) antibody, and 100% inhibition was determined as the signal from wells without biotinylated Fab0076 (i.e. corresponding to the assay background). Antibodies were considered to compete with 11F2 (mAb0005) for binding to FVII(a), if there was at least 50% observed inhibition (% inhibition) when the concentration of the antibody was tested in up to 10000-fold excess of the biotinylated Fab0076. Results are summarized in Table 8.

TABLE 8
Competition with 11F2-Fab0076 (mAb0005) for binding to FVII(a).

| Anti-FVII(a) antibody | % inhibition |
|---|---|
| mAb0765 | Yes |
| mAb0100 | No |
| mAb0047 | No |
| mAb0759 | Yes |
| mAb0762 | No |
| mAb0764 | No |

As described in Example 7, a cutoff of 50% was used to categorize antibodies as competitors or non-competitors.

### Example 8: FVIIa Activity Assay

FVIIa activity was measured using reagents from STAclot@VIIa-rTF kit (Diagnostica Stago) essentially as described in Morissey J H et al Blood, 1993; 81:734-44. The assay was performed on an ACLTOP500 automated coagulation instrument from Instrumentation Laboratory. In the assay was 40 μl diluted animal plasma sample, calibrator (recombinant FVIIa (rFVIIa) reference material calibrated against the international WHO reference) or quality control (QC) sample mixed with 40 μl FVII-deficient plasma and 40 μl soluble tissue factor (sTF) with phospholipids. A volume of 40 μl 25 mM CaCl2 was added to start the reaction, and the clotting time measured by the instrument. Sample and QC clot times were compared to rFVIIa calibration curves with the same concentration of plasma as the diluted sample to mitigate interference of plasma. The calibrator curve range was 5-1000 mIU/mL and was fitted using a third degree polynomial equation. QC and sample results were calculated by the software on the ACLTOP500 instrument. Results in IU/mL were converted to nM using the specific activities (IU/pmol) of the dosed compounds.

### Example 9: Pharmacokinetics in Rats of Recombinant FVIIa in Co-Formulation With Anti-FVII(a) Antibodies

Monoclonal anti-FVII(a) antibodies with desired in vitro properties from Example 5 were dosed IV to male Sprague Dawley rats in a co-formulation with 20 nmol/kg human FVIIa. The molar ratio of FVIIa to antibody was 1:1 or 1:5 as indicated in Table 9. During the experiment, the animals were allowed free access to feed and water. FVIIa plasma activity was measured using the FVIIa activity assay as described in Example 8.

Pharmacokinetic analysis was carried out by non-compartmental methods using WinNonlin. From the data the mean residence time (MRT) was calculated. Results are given in Table 9.

Among the antibodies with desirable properties from Example 5, mAb0005 (11F2) and mAb0001 (11F26) endowed human FVIIa with the longest mean residence times of 7.9 and 7.5 hours, respectively, in comparison to a free mean residence time of 1.1 hour for free human FVIIa (see Table 9). mAb0005 and mAb0001 (mAb0759) are high affinity antibodies (Example 6) exhibiting competition for binding to FVIIa (Example 7).

TABLE 9
Mean residence time (MRT) of FVIIa plasma activity in
sprague dawley rats following an IV dose of 20 nmol/kg
FVIIa in either a 1:1 or 1:5 molar ratio with antibody as indicated.

| Anti-FVII(a) | Molar ratio | |
|---|---|---|
| antibody | FVIIa:antibody | MRT (h) |
| Vehicle | n.a. | 1.1 |
| mAb0005 | 1:5 | 7.9 |
| mAb0007 | 1:1 | 3.9 |
| mAb0018 | 1:5 | 1.1 |
| mAb0004 | 1:5 | 3.6 |
| mAb0001 | 1:1 | 7.5 |
| mAb0587 | 1:1 | 4.0 |
| mAb0578 | 1:1 | 4.2 |

Vehicle indicates that FVIIa was administered in combination with buffer instead of antibody.

### Example 10: Effect of 11F2 Anti-FVII(a) Antibody on the Activation of FX by Free or TF-Complexed FVIIa

Preferably, anti-FVII(a) antibodies of the invention should not interfere with the pharmacological action of FVIIa on the membrane surface or initiation of coagulation, i.e. the activation of FX by the FVIIa-TF complex. To explore these aspects, the effect of 11F2 on the TF-independent and TF-dependent proteolytic activity of FVIIa was determined in the absence or presence, respectively, of lipidated TF. A one-armed monovalent antibody format was used to avoid avidity effects arising from the simultaneous binding of two FVIIa molecules to a normal bivalent antibody.

### Effect of 11F2 Antibody on FX Activation by FVIIa in the Presence of Phospolipid Membrane

Activity measurements were performed in assay buffer (50 mM HEPES, 100 mM NaCl, 10 mM CaCl2, pH 7.3, 0.1% PEG8000 and 1 mg/ml BSA) containing 10 nM FVIIa, 0 or 200 nM antibody (see Table 10), and 25 μM 25:75 phosphatidyl serine:phosphatidyl choline vesicles (Haematologic Technologies Inc.). Reactions were initiated by addition of 0-300 nM human plasma-derived factor X (FX) and allowed to incubate for 20 min at room temperature in a total reaction volume of 100 μl in a 96-well plate (n=2). After incubation, reactions were quenched by addition of 50 μl quench buffer (50 mM HEPES, 100 mM NaCl, 10 mM CaCl2, 80 mM EDTA, pH 7.3) followed by 50 μl 1 mM S-2765 chromogenic substrate (Chromogenix) in water. Conversion of the chromogenic substrate by the generated FXa was measured as the slope of the linear absorbance increase at 405 nm for 10 min using a Spectramax microplate spectrophotometer. By relating the measured slopes to those generated under similar conditions with known amounts of plasma-derived human FXa, initial rates of FXa generation on a molar basis could be estimated. Enzyme kinetic parameters were estimated by non-linear curve-fitting of the data to the Michaelis-Menten equation (v=kcat*[FX]*[FVIIa-TF]/(Km+[FX])) using GraphPad Prism.

As shown in Table 10, the monovalent 11F2 did not affect the rate of FX activation by free FVIIa in the presence of phospolipid vesicles.

### Effect of 11F2 Antibody on FX Activation by FVIIa in the Presence of TF

Activity measurements were performed in assay buffer (50 mM HEPES, 100 mM NaCl, 10 mM CaCl2, pH 7.3, 0.1% PEG8000 and 1 mg/ml BSA) containing 100 PM FVIIa, 0 or 200nM antibody (see Table 10), and 2 pM E. coli-derived TF fragment 1-244 incorporated into 25:75 phosphatidylserine:phosphatidy choline (PS:PC) vesicles as described by Smith and Morrissey (2005) J. Thromb. Haemost., 2:1155-1162. Reactions were initiated by addition of 0-30 nM human plasma-derived factor X (FX) and allowed to incubate for 20 min at room temperature in a total reaction volume of 100 μl in a 96-well plate (n=2). After incubation, reactions were quenched and FXa quantified as described above.

As shown in Table 10, monovalent 11F2 (mAb0077(OA)) did not affect the rate of FX activation by the FVIIa/TF complex.

TABLE 10
Effect of 200 nM 11F2 in monovalent format on the kinetic parameters for
activation of FX by the FVIIa-TF complex according to Example 10. Parameters shown as
mean ± SD (n = 3) were estimated by fitting to the Michaelis-Menten equation. k_{cat}/K_{M} is
shown relative to the value determined in the absence of antibody.

| | | Kₘ | k_{cat} | k_{cat}/K_{M} |
|---|---|---|---|---|
| 11F2 antibody | Activator | (nM) | (s⁻¹) | (%) |
| None | TF-FVIIa | 3.9 ± 0.6 | 1.5 ± 0.1 | 100 |
| mAb0077(OA) | TF-FVIIa | 3.9 ± 0.7 | 1.7 ± 0.1 | 110 ± 29 |
| None | FVIIa/PS:PC | 137 ± 15 | (2.8 ± 0.1) × | 100 |
| | | | 10-4 | |
| mAb0077(OA) | FVIIa/PS:PC | 89 ± 9 | (2.6 ± 0.1) × | 147 ± 5 |
| | | | 10-4 | |

### Example 11: Effect of 11F2 Antibody on the Inhibition of FVIIa by Plasma Inhibitors

FVIIa exhibits a relatively short half-life in circulation in part due to its inactivation by the abundant plasma inhibitor antithrombin (AT). Similarly, animal studies have implicated another plasma inhibitor, alpha-2-macroglobulin, in the inactivation of FVIIa. Using purified plasma-derived inhibitors, the effect of monovalent 11F2 antibody on the inactivation of FVIIa by these inhibitors was studied.

### Effect of 11F2 Antibody on the Inhibition of FVIIa by Antithrombin

The inhibition of FVIIa with human plasma-derived AT in the presence of monovalent 11F2 antibody (mAb0048, mAb0077(OA)) was performed under pseudo-first order conditions. The assay was conducted in a volume of 200 μl in assay buffer (50 mM HEPES, 100 mM Nacl, 10 mM CaCl2, 0.1% PEG8000, 1 mg/ml BSA, pH 7.3) at room temperature containing 200 nM FVIIa, 12 μM low molecular weight heparin (Enoxaparin, European Pharmacopeia Reference, Code E0180000, batch 5.0, Id 00CK18), and either 0 or 1000 nM antibody. Following 10 min pre-incubation, the reaction was initiated by addition of 5 μM AT (Antithrombin III, Baxter, Lot VNB1M007; repurified on heparin-sepharose column to remove serum albumin in the formulation). At selected time points, 10 μl aliquots were transferred into a total volume of 200 μl containing 0.5 mg/ml polybrene (Hexadimethrine bromide, Sigma, catalog no H9268, lot SLBC8683V), 200 nM sTF, and 1 mM S-2288 (Chromogenix) to quench the reaction and saturate FVIIa with sTF, which allowed for measurement of the residual FVIIa activity from the hydrolysis of the chromogenic substrate monitored at 405 nm for 5 min. Residual amidolytic activities were determined as the slope of the linear progress curves after blank subtraction and these were subsequently fitted to a first-order exponential decay function using GraphPad Prism software to derive pseudo-first order rate constants (kapp) for the reaction. An apparent second order rate constant (kinh) was estimated as the kapp divided by the AT concentration.

From this analysis, the rate of inhibition of FVIIa in the presence of monovalent 11F2 (mAb0077(OA)) was found to be 133±10 M-1s-1 as compared to 124±7 M-1s-1 for the inhibition of free FVIIa.

### Effect of 11F2 Antibody on the Inhibition of FVIIa by Alpha-2-Macroglobulin

The inhibition of FVIIa with human plasma-derived alpha-2-macroglobulin in the presence of 0 or 1000 nM monovalent 11F2 antibody (mAb0077(OA)) was performed under pseudo-first order conditions. The assay was conducted in a volume of 100 μl in assay buffer (50 mM HEPES, 100 mM NaCl, 5 mM CaCl2, 0.1% PEG8000, 0.01% Tween 80, pH 7.3) at room temperature containing 200 nM FVIIa and either 0 or 1000 nM antibody. The reaction was initiated by addition of 0 or 1000 nM alpha-2-macroglobulin purified from human plasma according to Banbula et al. (2005) J. Biochem., 138:527-537. At selected time points, 10 μl aliquots were transferred to 190 μl buffer (50 mM HEPES, 100 mM NaCl, 5 mM CaCl2, 0.1% PEG8000, 1 mg/ml BSA, pH 7.3) containing 200 nM sTF and 1 mM S-2288 (Chromogenix), which allowed for measurement of the residual FVIIa activity from the hydrolysis of the chromogenic substrate monitored at 405 nm for 5 min. Residual amidolytic activities were determined as the slope of the linear progress curves after blank subtraction and these were subsequently fitted to a first-order exponential decay function using GraphPad Prism software to derive pseudo-first order rate constants (kapp) for the reaction. An apparent second order rate constant (kinh) was estimated as the kapp divided by the alpha-2-macroglobulin concentration.

In the absence of antibody, the apparent second order rate constant for inhibition of FVIIa by alpha-2-macroglobulin was found to be 475±21 M-1s-1. However, in the presence of monovalent 11F2 antibody (mAb0077(OA)) no apparent inhibition of FVIIa was observed until the last time point at 125 min. From these studies, it can be concluded that 11F2 does not affect the inhibition of FVIIa by antithrombin, but protects FVIIa from inhibition by alpha-2-macroglobulin.

### Example 12: Effect of 11F2 Antibody on FVII Auto-Activation

Upon vascular injury, endogenous FVII binds with high affinity to its cofactor tissue factor (TF), which is exposed on the cells surrounding the vascular endothelium. During this process FVII is converted to FVIIa by limited proteolysis. Activation is believed to occur as a result of TF-mediated FVIIa-FVII trans-activation, also referred to as auto-activation. To determine the effect of 11F2 antibody on FVII auto-activation, activation of FVII was measured in the presence of lipidated TF, FVII, a limiting concentration of FVIIa, and monovalent 11F2 antibody.

Activity measurements were performed in assay buffer (50 mM HEPES, 100 mM NaCl, 5 mM CaCl2, pH 7.3 containing 0.1% PEG8000 and 1 mg/ml BSA) containing 2 nM FVIIa, 145 nM FVII, 0 or 200 nM monovalent 11F2 antibody (mAb0077(OA)). Reactions were initiated by addition of 2 nM lipidated E. coli-derived TF fragment 1-244 incorporated into 25:75 PS:PC vesicles as described by Smith and Morrissey (2005) J. Thromb. Haemost., 2:1155-1162. The total reaction volume was 100 μl. At selected time points (typically 5, 10, 15, 20, 30, 40, 50 and 60 min) generated FVIIa was quantified according to the sub-sampling procedure described below.

Quantification of FVIIa by sub-sampling-at selected time points 10 μl samples were quenched by transfer to 140 μl 50 mM HEPES, 100 mM NaCl, 5 mM EDTA containing 0.1% PEG8000, 1 mg/ml BSA and 215 nM soluble tissue factor (sTF). Following collection of all samples, FVIIa chromogenic activity was measured by addition of 50 μl S-2288 (4 mM) in 60 mM CaCl2. Conversion of the chromogenic substrate by the generated FVIIa was measured as the slope of the linear absorbance increase at 405 nm for 10 min using a Spectramax plate reader. By relating the measured slopes to those generated under similar conditions with known amounts of FVIIa, molar concentrations of FVIIa could be estimated.

FVIIa auto-activation was found to be TF-dependent, and is shown from the measured FVIIa activities in Table 11 to not being impaired by the presence of monovalent 11F2 antibody.

TABLE 11
TF-mediated autoactivation of FVIIa according to Example 12 in the
absence or presence of 200 nM monovalent 11F2 antibody mAb0077(OA)

| Time | Activity | Activity |
|---|---|---|
| (min) | no antibody | mAb0077(OA) |
| 5 | 5.5 ± 0.1 | 6.0 ± 0.1 |
| 10 | 9.2 ± 0.1 | 11.2 ± 1.3 |
| 20 | 23.6 ± 1.1 | 27.6 ± 1.8 |
| 30 | 45.6 ± 1.6 | 52.7 ± 3.6 |
| 40 | 71.1 ± 2 | 78.2 ± 4.1 |
| 50 | 87.8 ± 1.4 | 100.0 ± 5.4 |

### Example 13: Crystal Structure of 11F2-Fab0076 in Complex With Active-Site Inhibited FVIIa and Soluble Tissue Factor

To determine the epitope recognized by the murine antibody 11F2 on FVII(a), the corresponding Fab fragment (Fab0076) was crystallized in complex with H-D-Phe-Phe-Arg chloromethyl ketone (FFR-cmk; Bachem, Switzerland) active-site inhibited FVIIa (FVIIai) and the soluble tissue factor fragment 1-219 (sTF) using the hanging-drop method in accordance with Kirchhofer. D., et al., Proteins Structure Function and Genetics. (1995), 22, 419-425.

### Crystallisation

Crystals of Fab0076 mixed in a 1:1 molar ratio with FVIIai/sTF complex were grown using the hanging drop vapour diffusion technique at 18° C. A protein solution of 1 μl 4.6 mg/ml protein complex in 10 mM HEPES, 50 mM NaCl, 5 mM CaCl2, pH 7.0 was mixed with 0.5 μl 100 mM sodium citrate, pH 6.2 and 20% PEG 6000 as precipitant and incubated at a temperature of 18° C. over 1 ml of precipitant solution to obtain crystals of the complex.

### Diffraction Data Collection

A crystal was cryo protected in a solution consisting of 75 mM sodium citrate, pH 6.2, 15% PEG 6000, 4% glycerol, 4% ethylene glycol, 4.5% sucrose, and 1% glucose prior to flash cooling in liquid nitrogen. Diffraction data were collected at 100K at the MAX-lab (Lund, Sweden) beamline I911-3 using a marCCD225 detector from MAR Research. Autoindexing, integration and scaling of the data were performed with programmes from the XDS package (diffracting data statistics are summarised in Table 12).

### Structure Determination and Refinement

The structure was determined by molecular replacement using Phaser as implemented in the programme suite Phenix with the chains A and B of protein data bank entry 1YY8 and protein data bank entry 3ELA. The asymmetric unit contains two Fab:FVIIai/sTF complexes. The model was refined using steps of Phenix refinement and manual rebuilding in COOT. The refinement statistics are found in Table 12.

TABLE 12
Data collection and refinement statistics from
X-ray crystallographic structure determination of the
complex between active-site inhibited FVIIa,
soluble tissue factor (sTF) and Fab0076. Statistics for
the highest-resolution shell are shown in parentheses.

| | Fab0076/FVIIai/sTF |
|---|---|
| Wavelength (Å) | 1.0000 |
| Resolution range | 47.88 - 3.02 (3.128 - 3.02) |
| Space group | P 2₁ 2₁ 2₁ |
| Unit cell | 101.02 149.02 179.86 90 90 90 |
| Total reflections | 842454 (26830) |
| Unique reflections | 53490 (5172) |
| Multiplicity | 15.7 (5.1) |
| Completeness (%) | 99 (99) |
| Mean I/sigma(I) | 4.45 (0.39) |
| Wilson B-factor | 65.28 |
| R-merge | 0.8138 (4.973) |
| R-meas | 0.8409 (5.535) |
| R-pim | 0.2063 (2.382) |
| CC1/2 | 0.967 (0.14) |
| CC* | 0.992 (0.496) |
| Reflections used in | 53375 (5172) |
| refinement | |
| Reflections used for R- | 1995 (193) |
| free | |
| R-work | 0.2820 (0.4497) |
| R-free | 0.3393 (0.4832) |
| CC(work) | 0.897 (0.319) |
| CC(free) | 0.846 (0.303) |
| Number of non- | 15284 |
| hydrogen atoms | |
| macromolecules | 15224 |
| ligands | 59 |
| solvent | 1 |
| Protein residues | 1966 |
| RMS(bonds) | 0.003 |
| RMS(angles) | 0.59 |
| Ramachandran favored | 91 |
| (%) | |
| Ramachandran | 7.3 |
| allowed (%) | |
| Ramachandran outliers | 1.2 |
| (%) | |
| Rotamer outliers (%) | 1.6 |
| Clashscore | 2.37 |
| Average B-factor | 102.32 |
| macromolecules | 102.32 |
| ligands | 104.51 |
| solvent | 0.00 |
| Number of TLS groups | 78 |

The epitope (defined as residues in FVIIai having a non-hydrogen atom positioned within a distance of less than or equal to 4 Å from a non-hydrogen atom in Fab0076) was found to include the following residues according to SEQID NO:1:
R113
C114
H115
E116
G117
Y118
S119
L120
T130
V131
N184
T185
I186
P251
V252
E265
M391
R392
E394

The paratope (defined as residues in Fab0076 having a non-hydrogen atom positioned within a distance of less than or equal to 4 Å from a non-hydrogen atom in FVIIai) was found to include the following light chain residues:
Q27
G28
S30
D31
Y32
K49
Y50
Q53
H92
S93
F94

according to SEQID NO: 64

and heavy chain residues:
D32
Y54
N59
N101
Y102
Y103
G104
N105

according to SEQID NO: 63.

### Example 14: Humanization Murine 11F2

In order to humanize the murine antibody 11F2 (mAb0005) with VH and VL domain sequences corresponding to SEQ ID NO. 754 and SEQ ID NO. 750, respectively while conserving its high binding affinity for FVII(a), we combined information from sequence identity relative to human germlines, crystal structure of the complex between Fab0076 (Example 12) and active-site inhibited FVIIa (example 9) as well as in vitro binding data. Initially, we searched for human sequences of VH, VL, and VJ (for both heavy chain (HC) and light chain (LC)) with high sequence identity to the murine variable domain sequences of 11F2 in a database of human germlines using the blast algorithm. The sequences with highest sequence identity for VH of HC were: IGHV4-30-4*01, IGHV4-28*01, IGHV4-28*06, and IGHV459*01 and for the VJ-segment of HC the top germlines were: IGHJ5*01, IGHJ4*01. For the LC, the top sequences for VL were: IGKV6D-41*01 and IGKV3-11*01 and for the VJ-segment: IGKJ2*01and IGKJ2*02 (Table 12). Next, the differences between the human germlines and the murine VH and VL sequences were mapped onto the crystal structure of the Fab0076/FVIIa complex. Residues in the murine variable domains separated by a distance of more than or equal to 10 Å from residues in the epitope were anticipated to have no or little impact on binding affinity and were exchanged to the corresponding human germline amino acid entity. In turn, residues constituting the paratope were considered more problematic to substitute without affecting affinity and here the murine amino acid entity was preserved. A subset of residues near the binding interface that potentially could affect the binding affinity was identified. Humanized variants were generated by mutating residues distal to the epitope to the human amino acid entity from the germline alignment. Furthermore, residues close to the paratope were mutated to the human entity in a subset to make the variants as close to human germline as possible. Included in the set were variants were the murine CDRs are grafted on to a fully human germ line of HC and LC. From the initial analysis 12 LCs and 13 HCs were generated and paired into 23 variants according to Table 13.

Affinities for binding to FVIIa (listed in Table 13) were measured for the humanized antibodies using using Bio-Layer Interferometry (Fortebio). All steps were performed in running buffer (20 mM HEPES buffer (pH 7.4), 150 mM NaCl, 5 mM CaCl2, 0.03% Tween 20, 1 mg/ml IgG-free BSA) at 30° C. Antibodies were captured on anti-human tips (AHC, Fortebio) for 3 min at a concentration of 10 μg/ml. This was followed by a 3 min incubation to establish a baseline. Following this, association was monitored for 3 min using four different concentrations of FVIIa (25 nm, 50 nM, 100 nM, and 200 nM), followed by a 3 min dissociation. Sensorgrams were analysed using Fortebio data analysis software. The Fortebio data were used for ranking, and absolute affinity values may deviate from values determined by SPR (Example 6, Table 7).

TABLE 12
Human germline sequences used for humanization of murine
11F2. Numbers in parenthesis reflect identity ((n/m)
denotes n identical positions out of a total of m
positions) between the murine variable domains of Fab0076
and the specified human germline sequence

| VH-LC | VJ-LC | VH-HC | VJ-HC |
|---|---|---|---|
| IGKV3-11*01 | IGKJ2*01 | IGHV4-28*06 (87/98) | IGHJ5*02 |
| (81/95) | (11/11) | IGHV4-61*01 (90/99) | (12/12) |
| IGKV3D-11*01 | IGKJ2*012 | IGHV4-30-4*01 (91/99) | IGHJ1*01 |
| (80/89) | (11/11) | | (11/11) |

TABLE 13
Pairing of VH and VL in first round of humanization of the murine 11F2-
mAb0048 with measured affinities of corresponding antibodies for
FVIIa (determined as described herein). For some variants no
binding (nb) was measured.

| Antibody | SEQID No (VH) | SEQID No (VL) | K_{D} (nM) |
|---|---|---|---|
| mAb0108 | 74 | 70 | nb |
| mAb0109 | 82 | 78 | 11 |
| mAb0110 | 90 | 86 | 4.2E+4 |
| mAb0111 | 98 | 94 | nb |
| mAb0112 | 106 | 102 | 13 |
| mAb0113 | 114 | 110 | 1.8 |
| mAb0114 | 122 | 118 | 1.5 |
| mAb0115 | 130 | 126 | 1.5 |
| mAb0116 | 138 | 134 | 32 |
| mAb0117 | 146 | 142 | 1.4 |
| mAb0118 | 154 | 150 | 1.7 |
| mAb0119 | 162 | 158 | 1.0 |
| mAb0120 | 170 | 166 | 1.7 |
| mAb0121 | 178 | 174 | 2.0 |
| mAb0122 | 186 | 182 | 1.7 |
| mAb0123 | 194 | 190 | 5.8 |
| mAb0124 | 202 | 198 | 1.8 |
| mAb0125 | 210 | 206 | 1.7 |
| mAb0126 | 218 | 214 | 2.5 |
| mAb0127 | 226 | 222 | nb |
| mAb0128 | 234 | 230 | 1.7 |
| mAb0129 | 242 | 238 | 2.2 |
| mAb0130 | 250 | 246 | 1.8 |

Variants from the first round of humanization having an affinity on par or higher than the parental murine antibody were identified and those mutations that were found to preserve or improve binding affinity were used to design the second round of variants. From this set of mutations, the second round of variants were generated by inserting as many of these mutations on top of the humanised HC and LC already having the desired affinity. From the analysis 19 VH sequences (corresponding to SEQ ID No: 314, 514, 522, 530, 538, 546, 554, 562, 570, 578, 586, 594, 602, 610, 618, 626, 634, 642 and 650) and 25 VL sequences (corresponding to SEQ ID No: 310, 318, 326, 334, 342, 350, 358, 366, 374, 382, 390, 398, 406, 414, 422, 430, 438, 446, 454, 462, 470, 478, 486, 494 and 502) were designed and were experimentally tested by combining all VL with VL resulting in a total of 475 combinations. Dissociation constant (KD) values for binding to FVIIa were measured for the resulting 475 humanized antibodies using Bio-Layer Interferometry (Fortebio), as described above. Measured KD-values are listed in Table 14.

TABLE 14
Dissociation constant values (M, nb for no binding) for humanized variants of the
murine 11F2 anti-FVII(a) antibody. Antibodies are defined by their respective VH
and VL SEQID Nos.

| | VL | | | | | | | |
|---|---|---|---|---|---|---|---|---|
| VH | 310 | 318 | 326 | 334 | 342 | 350 | 358 | 366 |
| 314 | nb | 1.7E−08 | 1.5E−08 | 2.1E−08 | 2.8E−08 | 1.9E−08 | 1.8E−08 | nb |
| 514 | 3.1E−09 | 9.9E−10 | 7.5E−10 | 1.1E−09 | 1.3E−09 | 9.4E−10 | 8.7E−10 | 1.4E−09 |
| 522 | 8.3E−09 | 9.7E−10 | 8.1E−10 | 1.2E−09 | 1.4E−09 | 1.3E−09 | 1.3E−09 | 1.4E−08 |
| 530 | 2.5E−09 | 1.2E−09 | 1.1E−09 | 1.2E−09 | 1.2E−09 | 9.6E−10 | 1.0E−09 | 3.2E−09 |
| 538 | 2.5E−09 | 6.4E−10 | 5.8E−10 | 9.7E−10 | 1.2E−09 | 1.0E−09 | 8.9E−10 | 6.2E−09 |
| 546 | 3.2E−09 | 8.6E−10 | 8.1E−10 | 1.1E−09 | 1.2E−09 | 9.6E−10 | 9.9E−10 | 5.7E−09 |
| 554 | nb | 1.5E−08 | 1.4E−08 | 1.8E−08 | 2.3E−08 | 1.9E−08 | 1.6E−08 | nb |
| 562 | 6.4E−09 | 9.2E−10 | 7.8E−10 | 1.2E−09 | 1.3E−09 | 9.3E−10 | 9.5E−10 | 8.3E−09 |
| 570 | 1.3E−08 | 8.3E−10 | 5.5E−10 | 9.2E−10 | 1.5E−09 | 1.0E−09 | 8.5E−10 | 1.4E−08 |
| 578 | nb | 1.7E−08 | 1.6E−08 | 2.2E−08 | 4.3E−08 | 2.1E−08 | 1.9E−08 | nb |
| 586 | 1.5E−06 | 9.8E−09 | 9.9E−09 | 1.3E−08 | 1.8E−08 | 1.2E−08 | 1.1E−08 | 2.1E−08 |
| 594 | nb | 2.3E−08 | 2.2E−08 | 3.2E−08 | 1.2E−07 | 3.4E−08 | 2.4E−08 | nb |
| 602 | 4.8E−08 | 9.0E−09 | 8.8E−09 | 1.0E−08 | 1.4E−08 | 1.0E−08 | 9.2E−09 | 5.1E−08 |
| 610 | 8.0E−08 | 1.0E−08 | 1.1E−08 | 1.3E−08 | 1.6E−08 | 1.1E−08 | 1.1E−08 | 3.3E−07 |
| 618 | 1.2E−08 | 1.1E−09 | 1.1E−09 | 1.3E−09 | 1.2E−09 | 1.0E−09 | 9.5E−10 | 2.1E−08 |
| 626 | 9.2E−09 | 7.1E−10 | 7.9E−10 | 9.5E−10 | 8.5E−10 | 6.5E−10 | 7.2E−10 | 8.7E−08 |
| 634 | 2.1E−09 | 8.6E−10 | 9.0E−10 | 1.0E−09 | 1.0E−09 | 1.2E−09 | 8.2E−10 | 3.1E−09 |
| 642 | 1.6E−06 | 8.1E−08 | 2.3E−08 | 1.0E−06 | 4.0E−08 | 1.2E−07 | 3.2E−07 | nb |
| 650 | 1.6E−08 | 1.2E−09 | 1.1E−09 | 1.5E−09 | 1.6E−09 | 9.9E−10 | 1.1E−09 | 3.5E−08 |
| | VL | | | | | | | |
| VH | 374 | 382 | 390 | 398 | 406 | 414 | 422 | 430 |
| 314 | nb | 2.7E−08 | nb | nb | nb | nb | nb | nb |
| 514 | nb | 1.2E−09 | 4.7E−09 | 7.9E−09 | 5.3E−09 | 4.6E−09 | 6.8E−08 | 7.1E−09 |
| 522 | 1.4E−08 | 1.3E−09 | 1.5E−08 | 3.2E−07 | 1.7E−08 | 3.1E−08 | 1.1E−07 | 1.2E−08 |
| 530 | 4.2E−09 | 1.1E−09 | 4.1E−09 | 7.1E−09 | 4.3E−09 | 4.5E−09 | 2.6E−08 | 4.9E−09 |
| 538 | 7.4E−09 | 1.2E−09 | 7.0E−09 | 1.1E−08 | 6.1E−09 | 6.9E−09 | 2.1E−08 | 8.3E−09 |
| 546 | 6.8E−09 | 1.3E−09 | 7.0E−09 | 1.1E−08 | 6.7E−09 | 7.1E−09 | 2.4E−08 | 8.1E−09 |
| 554 | nb | 2.1E−08 | nb | nb | nb | nb | nb | nb |
| 562 | nb | 1.2E−09 | 1.0E−08 | nb | 7.7E−09 | 8.8E−09 | 1.8E−08 | 7.9E−09 |
| 570 | nb | 1.3E−09 | 4.8E−08 | 2.9E−08 | 2.0E−08 | 2.1E−08 | 2.4E−06 | 1.6E−08 |
| 578 | nb | 3.0E−08 | nb | nb | nb | nb | nb | 8.2E−07 |
| 586 | nb | 1.7E−08 | 3.8E−08 | nb | 1.9E−08 | 2.5E−08 | nb | 1.3E−08 |
| 594 | nb | 1.4E−07 | nb | nb | nb | nb | nb | nb |
| 602 | nb | 1.4E−08 | 3.7E−07 | nb | 9.0E−08 | 3.1E−07 | nb | 5.9E−07 |
| 610 | nb | 1.7E−08 | 3.8E−07 | nb | 4.8E−07 | 7.8E−07 | nb | 1.5E−06 |
| 618 | nb | 1.1E−09 | 3.3E−08 | 1.8E−07 | 2.4E−08 | 2.4E−08 | 5.8E−06 | 1.1E−07 |
| 626 | nb | 1.1E−09 | 2.7E−08 | 8.2E−07 | 8.7E−08 | 1.8E−08 | 2.4E−06 | 5.3E−08 |
| 634 | 4.5E−09 | 1.2E−09 | 4.3E−09 | 8.2E−09 | 4.2E−09 | 4.6E−09 | 2.6E−08 | 4.9E−09 |
| 642 | nb | 6.0E−07 | nb | nb | nb | nb | nb | 5.4E−07 |
| 650 | 4.6E−08 | 1.9E−09 | 9.9E−08 | 5.5E−08 | 1.8E−07 | 8.4E−08 | 2.6E−06 | 2.8E−08 |

| | VL | | | | | | | | |
|---|---|---|---|---|---|---|---|---|---|
| VH | 438 | 446 | 454 | 462 | 470 | 478 | 486 | 494 | 502 |
| 314 | nb | nb | 3.2E−08 | 2.6E−08 | 2.0E−08 | 6.4E−08 | 2.2E−08 | 2.8E−08 | 2.5E−08 |
| 514 | 5.4E−09 | 7.7E−09 | 1.3E−09 | 1.2E−09 | 1.2E−09 | 1.9E−09 | 9.5E−10 | 1.4E−09 | 9.2E−10 |
| 522 | 5.7E−08 | 1.7E−07 | 1.0E−09 | 1.2E−09 | 9.6E−10 | 1.6E−09 | 1.5E−09 | 1.7E−09 | 1.4E−09 |
| 530 | 4.6E−09 | 7.9E−09 | 1.1E−09 | 1.0E−09 | 9.6E−10 | 1.4E−09 | 9.9E−10 | 1.0E−09 | 9.4E−10 |
| 538 | 8.3E−09 | 1.2E−08 | 1.2E−09 | 1.3E−09 | 1.0E−09 | 1.8E−09 | 1.0E−09 | 1.1E−09 | 8.8E−10 |
| 546 | 8.0E−09 | 1.0E−08 | 1.1E−09 | 1.1E−09 | 1.2E−09 | 2.0E−09 | 1.1E−09 | 1.2E−09 | 1.1E−09 |
| 554 | nb | nb | 1.9E−08 | 2.0E−08 | 1.9E−08 | 2.1E−08 | 1.9E−08 | 2.2E−08 | 1.8E−08 |
| 562 | 1.1E−08 | nb | 1.4E−09 | 1.4E−09 | 1.4E−09 | 2.6E−09 | 1.3E−09 | 1.6E−09 | 1.1E−09 |
| 570 | 2.1E−07 | 2.4E−08 | 1.1E−09 | 1.1E−09 | 1.1E−09 | 1.9E−09 | 9.9E−10 | 1.1E−09 | 9.2E−10 |
| 578 | nb | nb | 2.5E−08 | 2.7E−08 | 2.5E−08 | 3.1E−07 | 2.3E−08 | 2.8E−08 | 2.4E−08 |
| 586 | 2.3E−08 | nb | 1.6E−08 | 1.8E−08 | 1.5E−08 | 2.7E−08 | 1.8E−08 | 1.7E−08 | 1.5E−08 |
| 594 | nb | nb | 3.2E−08 | 3.9E−08 | 3.5E−08 | 2.0E−07 | 3.3E−08 | 4.3E−08 | 3.0E−08 |
| 602 | 6.0E−07 | nb | 1.3E−08 | 1.5E−08 | 1.3E−08 | 2.4E−08 | 1.5E−08 | 1.4E−08 | 1.4E−08 |
| 610 | 6.2E−07 | 4.9E−08 | 1.6E−08 | 1.7E−08 | 1.6E−08 | 3.3E−08 | 1.7E−08 | 1.7E−08 | 1.7E−08 |
| 618 | 3.4E−08 | 2.0E−07 | 1.3E−09 | 1.4E−09 | 1.2E−09 | 2.0E−09 | 1.2E−09 | 1.4E−09 | 1.2E−09 |
| 626 | 7.7E−08 | 8.8E−07 | 1.4E−09 | 1.4E−09 | 1.1E−09 | 2.5E−09 | 1.6E−09 | 1.8E−09 | 1.8E−09 |
| 634 | 4.8E−09 | 7.7E−09 | 1.2E−09 | 1.2E−09 | 1.0E−09 | 1.8E−09 | 1.4E−09 | 1.3E−09 | 1.3E−09 |
| 642 | 3.3E−08 | nb | 2.5E−08 | 5.2E−08 | 4.7E−08 | 1.1E−08 | 7.7E−09 | 1.0E−08 | 4.6E−09 |
| 650 | 2.8E−08 | 3.9E−08 | 1.4E−09 | 1.6E−09 | 1.6E−09 | 3.0E−09 | 1.3E−09 | 1.6E−09 | 1.3E−09 |

### SPR Anelvele of Humanized 11F2 Variants

The affinity of selected humanized 11F2 variants were determined by SPR analysis as detailed in Example 6. Measured dissociation constants are listed in Table 15 and show retained high-affinity binding of the variants to human FVIIa.

TABLE 15
Estimated binding constants (K_{D}) for the interaction of humanized 11F2
antibodies in monovalent format and biAbs with FVIIa as determined by
surface plasmon resonance (SPR) analysis according to Example 6.

| Anti-FVII(a) antibody | K_{D} (M) |
|---|---|
| mAb0077(OA) | 4.1E−11 |
| mAb0099(OA) | 1.2E−10 |
| mAb0138(OA) | 1.3E−10 |
| mAb0140(OA) | 8.3E−11 |
| mAb0141(OA) | 7.8E−11 |
| mAb0142(OA) | 1.0E−09 |
| mAb0143(OA) | 7.9E−11 |
| mAb0705(OA) | 5.05E−11 |
| mAb0706(OA) | 5.42E−11 |
| mAb0707(OA) | 1.06E−10 |
| mAb0709(OA) | 1.04E−10 |
| mAb0710(OA) | 1.28E−10 |
| biAb0001 | 0.06E−09 |
| biAb0245 | 600E−09 |

### Functional Characterization of Humanized 11F2 Variants

The effect of selected humanized 11F2 variants on the activity of FVIIa and susceptibility to inhibition by antithrombin was determined as detailed in Example 5. Results are listed in Table 16 and show that the humanized variants retain desirable properties with respect to these parameters.

TABLE 16
Functional characterization of anti-FVII(a) antibodies in
thrombin generation (TGT) and antithrombin (AT)-inhibition
assays asdescribed in Example 5.

| | | AT |
|---|---|---|
| Anti-FVII(a) | TGT activity | inhibition |
| antibody | (% FVIIa) | (k_{inh %}) |
| mAb0077(OA) | 103 | 119 |
| mAb0099(OA) | 98 | 117 |
| mAb0137(OA) | 102 | 105 |
| mAb0138(OA) | 99 | 105 |
| mAb0139(OA) | 100 | 110 |
| mAb0140(OA) | 100 | 106 |
| mAb0141(OA) | 103 | 109 |
| mAb0142(OA) | 106 | 114 |
| mAb0143(OA) | 107 | 109 |
| mAb0705(OA) | 100 | 129 |
| mAb0706(OA) | 101 | 132 |
| mAb0707(OA) | 102 | 129 |
| mAb0709(OA) | 103 | 137 |
| mAb0710(OA) | 102 | 136 |

### Pharmacokinetics in Rats of FVIIa in Co-Formulation With Humanized 11F2 Variants

Humanized 11F2 variants family were dosed IV to male Sprague Dawley rats in a co-formulation with 20 nmol/kg FVIIa and as detailed in Example 9.

Results are given in Table 17 and show that several of the humanized 11F2 variants endow FVIIa with the same long half-life as the parental antibody.

TABLE 17
Mean residence time (MRT) of FVIIa plasma activity in
Sprague Dawley rats following an IV dose of 20 nmol/kg FVIIa in
different molar ratios with antibody. Values are mean ± SD,
n = 3.

| | Molar ratio | |
|---|---|---|
| Anti-FVII(a) antibody | FVIIa:antibody | MRT (h) |
| Vehicle | n.a. | 1.1 |
| mAb0005 | 1:5 | 7.9 |
| mAb0077(OA) | 1:1 | 11 ± 0.2 |
| mAb0099(OA) | 1:1 | 7.7 ± 0.4 |
| mAb0137(OA) | 1:1 | 7.0 |
| mAb0139(OA) | 1:1 | 2.7 |
| mAb0140(OA) | 1:1 | 7.7 |
| mAb0141(OA) | 1:1 | 7.7 |
| mAb0705(OA) | 1:1 | 7.7 ± 0.4 |
| mAb0706(OA) | 1:1 | 7.3 ± 0.3 |
| mAb0707(OA) | 1:1 | 6.4 ± 1.0 |
| mAb0709(OA) | 1:1 | 5.9 ± 0.4 |
| mAb0710(OA) | 1:1 | 5.7 ± 0.3 |

n.a.: Not appicable

### Example 15: Crystallisation and Epitope Mapping of 11F2 Fab0883 in Complex With Active-Site Inhibited FVIIa and Soluble Tissue Factor

To determine the epitope on FVII(a) recognized by humanized 11F2, i.e. mAb0705(OA) and mAb0842(OA), the corresponding Fab fragment Fab0883, was crystallized in complex with H-D-Phe-Phe-Arg chloromethyl ketone (FFR-cmk; Bachem, Switzerland) active-site inhibited FVIIa (FVIIai) and the soluble tissue factor fragment 1-219 (sTF) using the hanging-drop method in accordance with [Kirchhofer. D., et al., Proteins Structure Function and Genetics. (1995), 22, 419-425.

### Crystallisation

Crystals of a SEC purified complex of the Fab in complex with FVIIai/sTF were grown using the sitting drop vapour diffusion technique at 18° C. A protein solution of 360 nl protein complex (4.5 mg/ml) in 20 mM HEPES, 150 mM NaCl, 0.1 mM CaCl2, pH 7.4 was mixed with 360 nl of a precipitant solution containing 0.15 M CsCl and 15% (w/v) Polyethylen glycol 3350 and 360 nl water and equilibrated against 80 μl precipitant solution. Crystals grew within 6 weeks.

### Diffraction Data Collection

The crystal was cryo-protected in a solution consisting of 0.15M CsCl, 15% (w/v) Polyethylen glycol 3350 and 20% (v/v) glycerol prior to flash cooling in liquid nitrogen. Diffraction data were collected at 100K at a Rigaku FRX rotating anode generator equipped with a Dectris Pilatus 1M detector. Data reduction was performed with programmes from the XDS package (diffraction data statistics are summarised in Table 18).

### Structure Determination and Refinement

All crystallographic calculations were performed using the Phenix suite of crystallographic programs. The structure was determined by molecular replacement using the program Phaser with the coordinates of the complex structure obtained as described in example 13 as a search model. The asymmetric unit contains four Fab:FVIIai/sTF complexes. Iterative cycles of manual rebuilding using COOT and Phenix refinement yielded the final model (Table 18)

TABLE 18
Data collection and refinement statistics from X-ray structure
determination of the complex between active-site inhibited FVIIa,
soluble tissue factor (sTF) and Fab0883. Statistics for the
highest-resolution shell are shown in parentheses.

| | Fab0883 | |
|---|---|---|
| Wavelength | 1.542 | |
| Resolution range | 46.66 - 3.4 | (3.52 - 3.4) |
| Space group | P 1 21 1 ( No. 4) | |
| Unit cell | 144.78 100.79 181.74 | |
| | 90 101.23 90 | |
| Total reflections | 340556 | (24056) |
| Unique reflections | 69058 | (6446) |
| Multiplicity | 4.9 | (3.7) |
| Completeness (%) | 96.4 | (91.4) |
| Mean I/sigma(I) | 3.59 | (0.86) |
| Wilson B-factor | 63.67 | |
| R-merge | 0.31 | (1.05) |
| R-meas | 0.3465 | (1.22) |
| R-pim | 0.151 | (0.603) |
| CC1/2 | 0.98 | (0.697) |
| CC* | 0.995 | (0.906) |
| Reflections used in refinement | 68475 | (6424) |
| Reflections used for R-free | 1960 | (180) |
| R-work | 0.2968 | (0.4173) |
| R-free | 0.3323 | (0.4271) |
| CC(work) | 0.855 | (0.660) |
| CC(free) | 0.822 | (0.503) |

| | |
|---|---|
| Number of non-hydrogen atoms | 29262 |
| macromolecules | 29251 |
| ligands | 11 |
| Protein residues | 3791 |
| RMS(bonds) | 0.016 |
| RMS(angles) | 1.74 |
| Ramachandran favored (%) | 90.11 |
| Ramachandran allowed (%) | 7.73 |
| Ramachandran outliers (%) | 2.16 |
| Rotamer outliers (%) | 0.12 |
| Clashscore | 30.81 |
| Average B-factor | 57.11 |
| macromolecules | 57.11 |
| ligands | 68.66 |

### Epitope Mapping

A residue is considered to be part of the epitope, if all of the four independently determined FVIIa molecules in the crystal are having a non-hydrogen atom of said residue positioned within a distance of less than or equal to 4 Å from a non-hydrogen atom in the Fab. Thus, the epitope was found to comprise the following residues according to SEQ ID NO:1:
R113
C114
H115
E116
G117
Y118
S119
L120
T130
V131
N184
T185
P251
V252
V253
Q388
M391
R392

### Paratope Determination

A residue is considered to be part of the paratope, if all of the four independently determined Fab molecules in the crystal are having a non-hydrogen atom of said residue positioned within a distance of less than or equal to 4 Å from a non-hydrogen atom in FVIIa). The paratope was found to comprise the following light chain residues (according to SEQ ID NO: 814):
Q27
G28
Y32
Y50
H92
S93
F94

and heavy chain residues (according to SEQID NO: 818):
D32
Y54
Y103
N105

### Example 16: Hot-Spot Analysis of 11F2 mAb0842(OA) Using SPR

### Expression of Alanine Variants of FVIIa

hFVII alanine variants were generated with the QMCF Technology, a stable episomal expression system (Icosagen). CHOEBNALT85 cells were cultivated in Qmix1 medium (1L=1:1 CD-CHO and SFM II (NVO11514701)+10 ml Pen/Strep (Gibco, 15140-122)+2 ml Puromycin (Gibco, A11138-03)) in E125 flasks in a CO2 shaking incubator. On the day of transfection 1×10e7 CHOEBNALT85 cells were transfected with 2 μg hFVII variant-encoding plasmid and 50 μg salmon sperm DNA using electroporation (Bio-Rad Gene Pulser Xcell Electroporation System, 300 V, 900 μF, 4 mm cuvette). One day after transfection G418 selection was started by shifting cells to the Qmix2 medium (1 L=1:1 CD-CHO and SFM II (NVO11514701)+10 ml Pen/Strep (Gibco, 15140-122)+1 ml K-Vitamin (K.vit 13A 01311)+14 ml G418 (Gibco, 10131-027)). After 10-14 d of G418 selection cells reached>95% viability (Vi-Cell XR cell counter). Cells were split to 0.4×10e6 cells/ml to 2×250 ml Qmix2 in 2×E1000 flasks. After 3-4 days cells reached a density of approx. 4-5×10e6 cells/ml. Expression was initiated by adding 20% CHO CD Efficient Feed B (Gibco A10240)+6 mM GlutaMax (Gibco, 35050). After 4 days initiation another 10% CHO CD Efficient Feed B+6 mM GlutaMAX was added. On day 6 after initiation cultures were harvest and spun down (200 g, 5 min). Supernatants were collected and 15 mM HEPES (Gibco, 15630) and 5 mM CaCl2 (Sigma, 21115) were added. Supernatants were sterile-filtered using a 0.22 um bottle top filter (Corning, CLS430049).

Expression and Purification of cFVIIa-Chimera (22017-051)

cFVIIa-chimera was generated using a similar expression system as outlined above. The zymogen cFVII-chimera was purified from the medium using an affinity column prepared by coupling an in-house anti-FVII(a) antibody (F1A2) to sepharose beads as described in example 26. The anti-FVII(a) antibody F1A2 binds to the Gla domain of FVII(a) in a Ca++ dependent manner. The zymogen cFVII-chimera was activated using human FIXa and re-purified using the F1A2 affinity purification to obtain the final cFVIIa-chimera.

### Hot Spot Analysis

Hot-spot analysis using monovalent humanized antibody mAb0842(OA) was carried out by binding studies with a panel of 19 FVII(a) variants using surface plasmon resonance (Biacore T200) at 25° C. Anti-FVII(a) antibody (NN internal Ab 4F6 (Nielsen AL et al, PNAS 114(47) 12454-12459, 2017)), targeting the FVII(a) gla domain, at 25 μg/ml was immobilized on a CM4 sensor chip using standard amine coupling chemistry kit (both supplied by GE Healthcare). FVII(a) variants, according to Table 19, in cell culture supernatants (as described above) were diluted in running buffer and injected at a flow rate of 10 μl/min for 1 min to achieve a capture level of 5-55 RU. Each FVII(a) variant was captured by the immobilized anti-FVII(a) gla Ab. Subsequently, 540 nM (with 3× dilution) of mAb0842(OA) was injected at a flow rate of 30 μl/min for 7 min to allow for binding to captured FVII(a) variant followed by a 9 min buffer injection allowing dissociation of the one-armed anti-FVII(a) antibody. The running buffer was prepared by diluting 10-fold the 10×HBS-P buffer (supplied by GE Healthcare) and supplemented with 1 mg/ml BSA and 5 mM CaCl2 to give 10 mM HEPES, 150 mM NaCl, 0.05% v/v Polysorbate 20, pH 7.4, 5 mM CaCl2, 1 mg/ml bovine serum albumin (BSA). The running buffer was also used for dilution of anti-FVII(a) antibody and FVII(a) samples. Regeneration of the chip was achieved using 10 mM HEPES, 150 mM NaCl, 20 mM EDTA, 0.05 v/v Polysorbate 20, pH 7.4. Binding data were analysed according to a 1:1 Kinetic model and steady state analysis using BiaEvaluation 4.1 supplied by the manufacturer (Biacore AB, Uppsala, Sweden). Wherever possible, ka, kd and KD values from 1:1 kinetic fitting model are reported. For 4 FVII(a) variants, KD values using the steady state fitting model was reported. In addition, capture signal are reported for all FVII(a) variants. An amino acid residue is considered a hot-spot residue if substitution of that amino acid residue with alanine results in an affinity reduction of ten (10) fold or more relative to the wild type. Based on the data presented in Table 19 it is concluded that the amino acid residues H115, T130, V131, and R392 are hot-spots.

TABLE 19
FVII(a) variants binding interaction with the monovalent humanized
antibody mAb0842(OA) as determined by surface plasmon
resonance (SPR) analysis according to Example 16.

| | Capture | | Fold diff |
|---|---|---|---|
| FVII(a) variant | (RU) | K_{D} (nM) | (Variant/WT) |
| WT | 19.2 | 0.31 | 1.0 |
| R113A | 17.7 | 0.29 | 1.0 |
| H115A | 19.3 | 8.16 | 26.8 |
| G117A | 9.5 | 0.31 | 1.0 |
| Y118A | 54.8 | 1.48 | 4.9 |
| L120A | 15.2 | 0.60 | 2.0 |
| T130A | 16.9 | 32.10 | 105.2 |
| V131A | 18 | 5.22 | 17.1 |
| N184A | 8.4 | 1.91 | 6.3 |
| T185A | 15.4 | 1.01 | 3.3 |
| I186A | 28.2 | 0.39 | 1.3 |
| P251A | 9 | 0.85 | 2.8 |
| V252A | 15.6 | 0.28 | 0.9 |
| E265A | 8.3 | 0.98 | 3.2 |
| M391A | 5.5 | 0.25 | 0.8 |
| R392A | 14.1 | 43.40 | 142.3 |
| E394A | 26.9 | 0.79 | 2.6 |
| E116A | 9.5 | 0.50 | 1.6 |
| S119A | 51.6 | 0.31 | 1.0 |

### Example 18: Pharmacokinetics of Recombinant FVIIa in Co-Formulation With Humanized 11F2 Antibody in Cynomolgus Monkeys

FVIIa plasma activity-time profiles were estimated in cynomolgus monkey studies following IV or SC administration of either recombinant FVIIa (rFVIIa) alone or in a 1:3 molar ratio co-formulation with monovalent one-arm 11F2 mAb0705(OA). The formulations were administered as a single dose of 5.4 nmol/kg FVIIa (including 16.2 nmol/kg mAb0705(OA) for the co-formulation) with blood samples drawn over a three weeks period.

During the experiment the animals were kept and handled according to standard procedures of local health authorities and were allowed free access to feed and water. FVIIa plasma activity was measured using the FVIIa activity assay as described in example 8. Endogenous cynomolgus FVIIa was below LLOQ (0.1 nM) prior to administration and consequently disregarded.

Pharmacokinetic analysis of FVIIa plasma activity-time profiles was carried out by non-compartmental methods using Phoenix WinNonlin 6.4. From the data the following parameters were estimated: clearance (CL), mean residence time (MRT) and SC bioavailability (F). Parameters are listed in Table 20 and shows a substantial protraction of FVIIa activity by co-formulation with mAb0705(OA), both after IV and SC administration, compared to FVIIa in the absence of antibody.

TABLE 20
Clearance (CL), mean residence time (MRT) and SC bioavailability (F) of
FVIIa plasma activity in cynomolgus monkey studies following an IV or
SC dose of 5.4 nmol/kg FVIIa alone or in 1:3 molar ratio co-formulation
with mAb0705(OA). Values are mean (SD), n = 3.

| Formulation | CL (mL/h/kg) | MRT (h) | F (%) |
|---|---|---|---|
| FVIIa | 39 (5.2) | 2.0 (0.2) | 22 |
| FVIIa | 43.8 (2.2) | 2.15 (0.08) | Not determined |
| FVIIa:mAb0705(OA) | 2.7 (0.07) | 53.5 (15.3) | 61.4 |

### Example 19: Humanization and Optimization of the Murine Anti-TLT-1 Antibody mAb0012

### Humanization

The murine anti-human TLT-1 antibody mAb0082 disclosed in WO2012/117091 was used as the starting point for the humanization process. mAb0082 is derived from mAb0012, by incorporating two point mutations, C41A in VL and T61A in VH, to remove an unpaired cysteine in FR1 of VL and a N-glycosylation site in CDR2 of VH, respectively. The humanization process was based on standard molecular biology methods known to the person skilled in the art.

In brief, mAb0082 CDRH1, CDRH2 and CDRH3 sequences were grafted onto a human germline sequence based on the VH3_74/JH1 sequence as defined in the IMGT databases. Furthermore, three VH3_74/JH1 human amino acid substitutions were introduced into the grafted CDRH2 sequence in order to further humanize this CDR sequence: P62D, L64V and D66G. In order to obtain binding affinities comparable to that of mAb0082 three back mutations were introduced into the VL sequence at positions S49G, D62P and R98S. With respect to humanization of VL, mAb0082 CDRL1, CDRL2 and CDRL3 sequences were grafted onto a human germline sequence based on the VKII_A23/JK2 sequence as defined in the IMGT database. The VL sequence contains a potential deamidation hot-spot (NG motif) in CDR1. Using saturating mutagenesis at this position it was found that the NG motif could be eliminated by the N33Q substitutioin without compromizing affinity for TLT-1.

The final humanized and optimized variant of mAb0082 is referred to as mAb1076 corresponding to SEQID NO:934 (VL) and 938 (VH).

### SPR Analysis of Humanized TLT1 Variants

Binding of sTLT1 (corresponding to SEQ ID No: 3 with six histidine residues added at the C-terminus) to biAb from (Example 4) was probed by surface plasmon resonance (Biacore T200) at 25° C. Anti-human IgG was immobilised on a CM5 sensor chip (both supplied by GE Healthcare) using standard amine coupling chemistry. Purified biAbs according to Table 21 (1 nM) were injected at a flow rate of 10 μl/min for 1 min. Subsequently, sTLT1, between 0 and 60 μM, were injected at a flow rate of 30 μl/min for 3 min to allow for binding to the biAb followed by a 3 min buffer injection allowing for dissociation from the biAb. The running buffer was prepared by diluting 10-fold the 10×HBS-P buffer (supplied by GE Healthcare) and supplemented with 1 mg/ml BSA and 5 mM CaCl2 to give 10 mM HEPES, 150 mM NaCl, 0.05% v/v Polysorbate 20, pH 7.4, 5 mM CaCl2, 1 mg/ml bovine serum albumin (BSA). The running buffer was also used for dilution of biAb and sTLT1 samples. Regeneration of the chip was achieved using recommended regeneration buffer consisted of 3M MgCl2 (supplied by GE Healthcare). Binding data were analysed according to a 1:1 model using BiaEvaluation 4.1 supplied by the manufacturer (Biacore AB, Uppsala, Sweden). Analysis resulted in the binding constants reported in Table 21 demonstrating a range of affinity, from 2.9 nM to 320 nM, for sTLT1 binding by the biAbs.

TABLE 21
Estimated binding constants for the interaction of sTLT1 with
biAbs as determined by surface plasmon resonance (SPR)
analysis according to Example X.

| | Parental | Parental | K_{D} |
|---|---|---|---|
| biAb | anti-FVII(a) | Anti-TLT-1 | (M) |
| biAb0001 | mAb0865 | mAb1076 | 2.9E−9 |
| biAb0015 | mAb0865 | mAb1038 | 1.9E−8 |
| biAb0090 | mAb0865 | mAb1049 | 3.2E−7 |
| biAb0095 | mAb0865 | mAb1047 | 7.5E−8 |

### Example 20: Crystal Structure of Hz-TLT1 and TLT-1 Peptide Complex Preparation

The Fab fragment used for crystallization in complex with the TLT-1 stalk peptide comprised the VL and VH domain sequences corresponding to mAb1076 (SEQ ID NO: 854 and 858, respectively), a human IgG4 CH1 domain, and a human kappa CL with a single point mutation (G157C). The G to C substitution is in the constant domain of the Fab fragment, i.e. far from the antigen binding site, and has no influence on the binding to TLT-1. The 37-mer stalk peptide, EEEEETHKIGSLAENAFSDPAGSANPLEPSQDEKSIP (SEQ ID NO: 13), corresponding to residues 111-147 of SEQ ID No: 2, was prepared by standard peptide synthesis methods known to the person skilled in the art. The Fab and the stalk peptide were mixed at a 1:2 molar ratio in hepes buffer (20 mM Hepes (pH 7.3), 150 mM of NaCl). The 1:1 Fab:peptide complex was isolated using gelfiltration on a superdex 200 column eluted with hepes buffer and was subsequently concentrated to ˜11 mg/mL and used for crystallization.

### Crystallisation

Crystals of the gel filtrated 1:1 molar Fab/peptide complex were grown using the sitting drop vapour diffusion technique at 18° C. A protein solution of 150 nl 10.8 mg/ml Fab:peptide complex in 20 mM Hepes, pH 7.3 and 150 mM NaCl was mixed with 50 nl of 1M LiCI, 0.1M NaCitrate-citrid acid, pH 4 and 20% (w/v) PEG 6000 as precipitant and incubated over 60 μl precipitant.

### Diffraction Data Collection

The crystal was cryo protected by addition of 1 μl of precipitant added 20% of ethylene glycol to the crystallisation drop prior to flash cooling in liquid nitrogen. Diffraction data were collected at 100K at the BioMAX beamline at the MAX IV synchrotron (Lund, Sweden), using an Eiger 16M Hybrid-pixel detector from Dectris. Autoindexing, integration and scaling of the data were performed with programmes from the XDS package (diffraction data statistics are summarised in Table 22).

### Structure Determination and Refinement

The asymmetric unit contains two Fab:peptide complexes as judged from Matthews coefficient analysis. The structure was determined by molecular replacement. Phaser as implemented in the programme suite Phenix was used with the chains H and L of protein data bank entry 5KMV as search model localising two Fabs. These were model built with the correct amino acid sequence using COOT and thereafter refined using Phenix refinement. Amino acids 7 to 21 from the peptide were clearly seen in the difference electron density maps and could be model built manually using COOT. The model was further refined using steps of Phenix refinement and manual rebuilding in COOT. The refinement statistics are found in Table 22.

TABLE 22
Data collection and refinement statistics.

| Wavelength (Å) | 0.9799 | |
|---|---|---|
| Resolution range (Å) | 29.42-1.49 | (1.543-1.49) |
| Space group | P 1 | |
| Unit cell (Å, deg) | 53.23 65.38 67.15 | |
| | 91.88 91.72 92.89 | |
| Total reflections | 260187 | (25653) |
| Unique reflections | 140071 | (9127) |
| Multiplicity | 1.9 | (1.9) |
| Completeness (%) | 91.03 | (62.11) |
| Mean I/sigma(I) | 9.03 | (1.37) |
| Wilson B-factor | 18.72 | |
| R-merge | 0.05225 | (0.593) |
| R-meas | 0.07387 | (0.8385) |
| R-pim | 0.05222 | (0.5928) |
| CC1/2 | 0.994 | (0.0683) |
| CC* | 0.999 | (0.358) |
| Reflections used in | 134361 | (9125) |
| refinement | | |
| Reflections used for R- | 1784 | (123) |
| free | | |
| R-work | 0.1582 | (0.2655) |
| R-free | 0.1779 | (0.2932) |
| CC(work) | 0.966 | (0.671) |
| CC(free) | 0.962 | (0.632) |

| | |
|---|---|
| Number of non- | 8094 |
| hydrogen atoms | |
| macromolecules | 7042 |
| Solvent | 1052 |
| Protein residues | 897 |
| RMS(bonds) | 0.009 |
| RMS(angles) | 1.36 |
| Ramachandran favored | 97.06 |
| (%) | |
| Ramachandran allowed | 2.83 |
| (%) | |
| Ramachandran outliers | 0.11 |
| (%) | |
| Rotamer outliers (%) | 1.35 |
| Clashscore | 4.17 |
| Average B-factor (Å²) | 25.71 |
| macromolecules | 24.26 |
| Solvent | 35.47 |
| Number of TLS groups | 1 |

Statistics for the highest-resolution shell are shown in parentheses.

Statistics for the highest-resolution shell are shown in parentheses.

### The Epitope and Paratope of the Fab/Peptide Complex

The epitope is defined as residues in the TLT-1 stalk-peptide characterized by having a heavy atom (i.e. a non-hydrogen atom) within a distance of 4.0 Å from a heavy atom in the Fab in both of the complexes in the asymmetric unit. Similarly, the paratope is defined as residues in the Fab fragment characterized by having a heavy atom within a distance of 4.0 Å from a heavy atom in the TLT-1 stalk-peptide in both of the complexes in the asymmetric unit the epitope was found to comprise the following residues from the 37aa TLT-1 peptide according to SEQ ID NO: 13:
K8
I9
G10
S11
L12
A13
N15
A16
F17
S18
D19
P20
A21

corresponding to K118, I119, G120, S121, L122, A123, N125, A126, F127, S128, D129, P130 and A131 of SEQ ID No:2 and 3).

The paratope comprises the following residues from the heavy chain variable domain (SEQ ID NO: 938):
V2
F27
R31
Y32
W33
E50
T57
N59
S98
G99
V100
T102
S103

and from the light chain variable domain (SEQ ID NO: 934):
H31
Y37
H39
Y54
F60
S61
S96
T97
V99
Y101

### Example 21: Effect of Affinity on Anti-FVII(a)/Anti-TLT-1 Bispecific Antibody Stimulatory Activity

To determine the effect of affinity on bispecific antibody activity, a number of anti-FVII(a) and anti-TLT-1 mAbs from the humanization of 11F2 mAb0005 (see Example 14) and mAb0012 (see Example 19) and with varying affinities for FVIIa and TLT-1, respectively, were tested in a bispecific format in a FXa generation assay using lipidated TLT-1 as described in WO2011/023785.

In a first step, FX activation was measured in the presence of 4 nM recombinant TLT-1 incorporated into 10:90 phosphatidyl serine:phosphatidyl choline vesicles (WO2011023785), 2.5 nM FVIIa, and bispecific antibody (biAb) in a concentration series from 0 to 300 nM. Following 10-min preincubation in assay buffer (50 mM HEPES, 100 mM NaCl, 10 mM CaCl2, pH 7.3 +1 mg/ml BSA and 0.1% PEG8000) at room temperature, 150 nM plasma-derived FX (Haematologic Technologies) were added to give a total volume of 50 μl and activation allowed to proceed for 20 min. Activation was then terminated by addition of 25 μl quench buffer (50 mM HEPES, 100 mM NaCl, 80 mM EDTA, pH 7.3) and generated FXa quantified from its ability hydrolyze 0.5 mM S-2765 (Chromogenix) chromogenic substrate (added as 2 mM stock in 25 μl volume), which was followed at 405 nm for 5 min in a SPECTRAmax Plus384 plate reader. From the slope of the linear absorbance increases, the normalized activity (A_{biAb}) was calculated for each biAb at a concentration of 100 nM by subtraction of the background activity in the absence of biAb and division by the FVIIa concentration in the assay.

In a second step, the same assay was run with biAb replaced by assay buffer and a concentration series of FVIIa from 0 to 80 nM. The slope of the linear relationship between background-subtracted FXa generation and the concentration of FVIIa in the assay provided a measure of the specific activity of free FVIIa (A_{FVIIa}) under the assay conditions employed.

Based on the measured activities, the stimulatory activity of each biAb at 100 nM was calculated as the ratio A_{biAb}/A_{FVIIa}. The stimulatory activity provides a measure of the fold increase in generated FXa by FVIIa upon addition of 100 nM biAb.

Stimulatory activities are provided in Table 23 and shows a dependence of the biAb stimulation on the strengths (expressed as dissociation constant (KD) values) by which FVIIa and TLT-1 are bound, respectively. Among the biAbs tested, biAb0001 exhibit the highest stimulatory activity.

TABLE 23
Stimulatory activities of bispecific antibodies in the presence FX,
FVIIa, and lipidated TLT-1 as described in Example 21. For each
bispecific antibody, the measured stimulatory activity is
listed (mean ± SD, n = 2) together with dissociation constants for
the interaction with FVIIa and TLT-1, respectively.

| Bi- | Anti- | Anti- | KD | K_{D} | Stimu- |
|---|---|---|---|---|---|
| specific | FVIIa | TLT-1 | (FVIIa) | (TLT-1) | lation |
| antibody | arm | arm | [nM] | [nM] | (fold) |
| biAb0001 | mAb0865 | mAb1076 | 0.06 | 2.9 | 29.2 ± 1.7 |
| biAb0015 | mAb0865 | mAb1038 | 0.06 | 19 | 18.7 ± 1.5 |
| biAb0095 | mAb0865 | mAb1047 | 0.06 | 75 | 13.9 ± 1.1 |
| biAb0090 | mAb0865 | mAb1049 | 0.06 | 320 | 11.1 ± 1 |
| biAb0011 | mAb0875 | mAb1076 | 0.28 | 2.9 | 20.3 ± 1.3 |
| biAb0016 | mAb0875 | mAb1038 | 0.28 | 19 | 17.3 ± 1.6 |
| biAb0096 | mAb0875 | mAb1047 | 0.28 | 75 | 12.4 ± 1.1 |
| biAb0091 | mAb0875 | mAb1049 | 0.28 | 320 | 11.3 ± 1.3 |
| biAb0012 | mAb0872 | mAb1076 | 2.2 | 2.9 | 22.1 ± 0.4 |
| biAb0017 | mAb0872 | mAb1038 | 2.2 | 19 | 13.6 ± 0.1 |
| biAb0097 | mAb0872 | mAb1047 | 2.2 | 75 | 17.4 ± 0.1 |
| biAb0092 | mAb0872 | mAb1049 | 2.2 | 320 | 11.2 ± 0.2 |
| biAb0013 | mAb0874 | mAb1076 | 16.2 | 2.9 | 16 ± 0.1 |
| biAb0018 | mAb0874 | mAb1038 | 16.2 | 19 | 12.8 ± 0.2 |
| biAb0098 | mAb0874 | mAb1047 | 16.2 | 75 | 15.3 ± 0.1 |
| biAb0093 | mAb0874 | mAb1049 | 16.2 | 320 | 9.6 ± 0.2 |
| biAb0014 | mAb0873 | mAb1076 | 600 | 2.9 | 8 ± 0.2 |
| biAb0019 | mAb0873 | mAb1038 | 600 | 19 | 9.4 ± 0.5 |
| biAb0099 | mAb0873 | mAb1047 | 600 | 75 | 5.8 ± 0.5 |
| biAb0094 | mAb0873 | mAb1049 | 600 | 320 | 5.4 ± 0.5 |

### Example 22: Effect of Epitope Location on Anti-FVII(a)/Anti-TLT-1 Bispecific Antibody Stimulatory Activity

To determine the effect of epitope location on bispecific antibody activity, a number of anti-TLT-1 and anti-FVIIa mAbs binding to different epitopes on TLT-1 and FVIIa, respectively, were tested in a bispecific format in a FXa generation assay as done in Example 21.

Results are provided in Table 24 and shows a dependence of the biAb stimulatory activity on epitope location. In particular, anti-TLT-1 mAbs mAb1076, mAb0023, mAb0051, and mAb0062 in combination with anti-FVIIa mAb mAb0865 exhibit comparable stimulatory activity.

TABLE 24
Stimulatory activities of bispecific antibodies in the presence FX,
FVIIa, and lipidated TLT-1 as described in Example 22. For each
bispecific antibody, the measured stimulatory activity activity is
listed (mean ± SD, n = 2) together dissociation constants for the
interaction with FVIIa and TLT-1, respectively.

| | Anti-FVIIa | Anti-TLT-1 | Stimulation |
|---|---|---|---|
| biAb | arm | arm | (fold) |
| biAb0001 | mAb0865 | mAb1076 | 29.2 ± 1.7 |
| biAb0241 | mAb0865 | mAb0023 | 35.3 ± 2.4 |
| biAb0242 | mAb0865 | mAb0051 | 21.8 ± 0.9 |
| biAb0243 | mAb0865 | mAb0062 | 25.5 ± 2.0 |
| biAb0244 | mAb0864 | mAb1076 | 2.7 ± 0.2 |

### Example 23: Antigen Assay for Human IgG (LOCI)

The presence of human IgG (hIgG) in cynomolgus plasma was measured by Luminescent Oxygen Channelling Immunoassay (LOCI). In brief, the LOCI reagents included two latex bead reagents (donor and acceptor beads) and a biotinylated monoclonal antibody against hIgG (Biosite, cat. no. AFC4249). The donor bead reagent, containing a photosensitive dye, was coated with streptavidin. The second bead reagent, the acceptor beads, was conjugated with an in-house monoclonal antibody (0421) against hIgG, which made up the sandwich. During the assay, the three reactants were combined with hIgG in the plasma to form a bead-aggregate immuno complex. Excitations of the complex released singlet oxygen molecules from the donor beads, which were channelled into the acceptor beads and triggered a chemiluminescence response. This was then measured in the EnVision plate reader. The amount of light generated, reported as counts per second (cps), was proportional to the concentration of hIgG. Samples were diluted at least 100× in assay buffer and a calibration curve was prepared based on hIgG added to 1% cynomolgus plasma.

### Example 24: Antigen Assay for FVII(a) (LOCI)

FVII(a) antigen including FVII zymogen, FVIIa, and FVIIa:antithrombin (FVIIa:AT) complexes was measured by a LOCI assay as described in example 23, except that the assay for FVII(a) consisted of acceptor beads coated with an in-house anti-FVII(a) antibody (4F9) and an in-house biotinylated anti-FVII(a) monoclonal antibody (4F7). Samples were diluted at least 100× in assay buffer and a calibration curve was prepared by adding known amounts of human rFVIIa to assay buffer.

### Example 25: Antigen Assay for FVIIa:AT (Antithrombin) (EIA)

FVIIa:AT (antithrombin) complexes were measured by use of an enzyme immuno assay (EIA) as described in Agersø H et al, J Thromb Haemost 2011; 9:333-8. A monoclonal anti-FVIIa antibody (Dako Denmark A/S, Glostrup, Denmark, product code O9572) that binds to the N-terminal EGF-domain and does not block antithrombin binding was used for capture of the FVIIa:AT complex. A preformed complex of human FVIIa and cynomolgus antithrombin (FVIIa:AT) was prepared by incubation of FVIIa with 2-fold molar excess of antithrombin in the presence of 10 μM low-molecular weight heparin (Enoxaparin). The residual amidolytic activity of FVIIa (see Example 11) after overnight incubation at room temperature was verified to be below 10% of the initial FVIIa activity. The complex was used to construct EIA calibration curves. A polyclonal anti-human antithrombin antibody peroxidase conjugate was used for detection (Siemens Healthcare Diagnostics ApS, Ballerup, Denmark, product code OWMG15). TMB was added, colour development allowed to proceed until sufficient colour was developed, stopped by adding H2SO4 and the absorbance at 450 nm with 650 nm as reference measured on an absorbance plate reader (BioTek). The colour intensity is proportional to the concentration of FVIIa:AT.

### Example 26: Preparation of Human FVIIa, FVII (Zymogen), and FVIIa:AT (Antithromb in) Complex

### Preparation of Human FVIIa (Activated FVII)

Unless otherwise noted, recombinant human activated FVII (FVIIa) was prepared as described in Thim et al. (1988) Biochemistry 27:7785-7793 and Persson et al. (1996) FEBS Lett 385:241-243.

### Preparation of Human FVII (Zymogen FVII)

Recombinant human FVII produced in CHO cells were purified by single-step calcium-dependent affinity chromatography as described by Thim et al. (1988) Biochemistry 27:7785-7793. Following purification, zymogen FVII was dialyzed into 10 mM MES, 100 mM NaCl, 10 mM CaCl2, pH 6.0 buffer. The level of activated FVII (FVIIa) in the preparation was determined by measuring the amidolytic activity in the presence of 1 mM chromogenic substrate S-2288 and 200 nM sTF (see Example 11). By relating this to a standard curve prepared with known concentration of FVIIa, the measured activity could be converted to a molar concentration of FVIIa in the zymogen FVII preparation.

### Preparation of Human FVIIa:AT (Antithrombin) Complex

FVIIa:AT (antithrombin) complex was prepared by incubating equimolar concentrations of human recombinant FVIIa, human plasma-derived AT (Baxter), and low-molecular weight heparin (Enoxaparin sodium) for 16 hours at 4° C. To remove contaminating excipients, AT was re-purified before use on heparin sepharose 6 fast flow (GE healthcare) column by applying a sodium chloride gradient. Eluted AT was up-concentrated by ultrafiltration to give a final preparation in 10 mM HEPES, 25 mM NaCl, pH 7.3 containing 50% glycerol. The FVIIa:AT complex was purified by SEC chromatography at 4° C. in 20 mM MES, 100 mM NaCl, 1 mM EDTA, pH 5.5 to maximize complex stability. Residual levels of FVIIa in the preparation were determined as described above. To minimize complex breakdown, preparations were stored in aliquots at −80° C. and immediately thawed and kept on ice before use.

### Example 27: Single Dose Pharmacokinetics of Anti-FVII(a)/Anti-TLT-1 biAb in Cynomolgus Monkeys

biAb0001 and the corresponding YTE-variant, biAb0352, where additional three half-life extending substitutions (M252Y, S254T and T256E) were introduced in to the constant heavy chain domain, were dosed intravenously (iv) at 3.0, 9.49 or 30 nmol/kg or subcutaneously (sc) at 9.49 nmol/kg to cynomolgus monkeys. Each group consisted of two monkeys (one male and one female). Sodium citrate-stabilised blood samples of 1 mL were taken prior to dosing and for the iv groups at 0.5 h; 2.5 h; 6 h; 12 h; 24 h; 48 h; 72 h and at day 8; 10 and 15 after dosing. For the sc groups blood samples were taken pre-dose and at 0.5 h; 3 h; 6 h; 12 h; 16 h; 24h; 30h, 38h; 48 h; 54 h; 72 h; 78 h; 96 h; 120 h; and at day 8, 10 and 15 after dosing. Blood samples were centrifuged 10 min at 2000 g, the plasma removed, divided into aliquots and stored at 80° C. until analysis for hIgG (see example 23), total FVII(a) antigen (see example 24), FVIIa activity (see example 8) and FVIIa:AT complexes (example 25). Pharmacokinetic (PK) analysis of concentration versus time profiles of hIgG was carried out by non-compartmental methods using Phoenix WinNonlin 6.4. The following PK parameters are shown in Table 24: half-life (t½), clearance (Cl), volume of distribution (Vz), mean residence time (MRT) and SC bioavailability (F).

TABLE 24
PK parameters of biAb with and without YTE mutation analysed
based on plasma samples from cynomolgus monkeys.*

| | Dose | | | | | | |
|---|---|---|---|---|---|---|---|
| | (nmol/ | | t_{1/2} | Cl or Cl/F** | V_{z} | MRT | AUC |
| Compound | kg) | Route | (h) | (mL/h × kg) | (mL/kg) | (h) | (h × nM) |
| biAb | 3.0 | iv | 80 | 0.64 | 73.6 | 106 | 4084 |
| 0001 | | | 100 | 0.47 | 67.7 | 132 | 5325 |
| | 9.49 | iv | 127 | 0.43 | 78.3 | 177 | 16269 |
| | | | 121 | 0.33 | 58.1 | 161 | 21647 |
| | 30 | iv | 207 | 0.48 | 143.4 | 305 | 33046 |
| | | | 207 | 0.36 | 106.9 | 277 | 49476 |
| | 9.49 | sc | 127 | 0.70 | — | 193 | 9672 |
| | | | 138 | 0.43 | — | 205 | 15059 |
| biAb | 3.0 | iv | 98 | 0.27 | 37.9 | 133 | 8445 |
| 0352 | | | 212 | 0.21 | 65.7 | 285 | 8040 |
| | 9.49 | iv | 218 | 0.20 | 63.5 | 296 | 26397 |
| | | | 201 | 0.26 | 75.0 | 267 | 21870 |
| | 30 | iv | 206 | 0.26 | 78.2 | 282 | 65712 |
| | | | 226 | 0.27 | 87.4 | 304 | 62075 |
| | | | 249 | 0.31 | — | 376 | 13966 |
| | 9.49 | sc | 218 | 0.33 | — | 342 | 13932 |

*each line represents data from one cynomolgus monkey,
**Cl is noted for the iv dosed groups, and Cl/F for the sc groups. F corresponds to sc bioavailability.

Accumulation of endogenous FVII(a) was observed in the monkeys dosed with BiAb0001 or the corresponding YTE-variant, biAb0352. Table 25 shows pre-dose levels of FVII(a) antigen, FVIIa activity and FVII:AT together with mean accumulated levels measured between 72 and 240 h after administration. An antibody dose-dependent accumulation of FVII(a) antigen, FVIIa activity and FVIIa:AT was observed. FVII(a) antigen was increased up to 3-fold relative to the pre-dose level, and FVIIa and FVIIa:AT up to 5-fold. The data demonstrate that iv or sc administration of a single dose of biAb0001 or biAb0352 with YTE mutation lead to accumulation of endogenous FVII(a) antigen, FVIIa activity and FVIIa:AT complexes in vivo.

TABLE 25
Accumulation of FVII(a) antigen, FVIIa and FVIIa:AT after
single iv or sc administration of biAb with or without
YTE mutations to cynomolgus monkeys.

| | | | Level achieved between 72 and | | |
|---|---|---|---|---|---|
| | Route | | 240 h after dosing* | | |
| | of | Dose | FVII(a) | FVIIa | |
| | admini- | (nmol/ | antigen | activity | FVIIa:AT |
| Compound | stration | kg) | (nM) | (nM) | (nM) |
| biAb0001 | iv | 3 | 11.2 ± 2.7 | 0.21 ± 0.05 | 1.98 ± 0.44 |
| biAb0001 | iv | 9.49 | 15.9 ± 2.4 | 0.59 ± 0.20 | 4.67 ± 1.15 |
| biAb0001 | iv | 30 | 19.7 ± 4.8 | 0.91 ± 0.56 | 8.50 ± 0.62 |
| biAb0001 | sc | 9.49 | 14.6 ± 3.4 | 0.43 ± 0.13 | 3.36 ± 0.68 |
| biAb0352 | iv | 3 | 8.9 ± 2.6 | 0.37 ± 0.16 | 3.21 ± 1.42 |
| biAb0352 | iv | 9.49 | 17.6 ± 4.5 | 0.66 ± 0.12 | 4.62 ± 0.64 |
| biAb0352 | iv | 30 | 17.6 ± 4.5 | 2.04 ± 1.19 | 8.05 ± 1.15 |
| biAb0352 | sc | 9.49 | 20.4 ± 3.0 | 0.81 ± 0.15 | 3.88 ± 0.27 |
| Pre-dose | | | 6.2 ± 1.4 | 0.16 ± 0.04 | 1.63 ± 0.37 |

*data are mean and SD of measurements from n = 2 animals per group and 3 time points from the iv groups and 6 time points from the sc groups. The pre-dose values are based on one time-point (pre-dose) for all 16 animals.

### Example 28: Single and Multiple Dose Pharmacokinetics of Monovalent 11F2 Anti-FVII(a) Antibody mAb0705(OA) in Cynomolgus Monkeys

In vivo accumulation of FVII, FVIIa and FVIIa:AT was analysed by administration of the monovalent 11F2 anti-FVII(a) antibody mAb0705(OA) to cynomolgus monkeys, followed by measurement of FVII antigen, FVIIa activity and FVIIa:AT in plasma samples. Two male cynomolgus monkeys of approx. 2.5 kg were dosed iv in the saphenous, cephalic or lateral caudal veins of the tail with 40 nmol/kg mAb0705(OA), and 3 male cynomolgus monkeys were dosed every other day for two weeks subcutaneously (sc) in the thigh (alternating between left and right thigh) with 20 nmol/kg of the one-armed anti-FVIIa antibody (i.e. the antibody was dosed on day 1, 3, 5, 7, 9, 11, 13, and 15). Blood was sampled in 3.8% trisodium citrate from the cephalic vein or the femoral vein at time points up to 21 days after dosing.

The blood was centrifuged 10 min at 2300 g and plasma aliquoted and stored at −80° C. until analysis for hIgG (see example 23), total FVII(a) antigen (see example 24), FVIIa activity (see example 8) and FVIIa:AT complexes (see example 25). PK analysis of concentration versus time profiles was carried out by non-compartmental methods using Phoenix WinNonlin 6.4. The PK parameters for iv administration are shown in Table 26. The half-life (t½) of the one-armed antibody was on the average 116 h (4.8 days) after single iv administration. The estimated half-life for the sc administered antibody was 181±20 h (mean and SD of n=3). The antibody accumulated during the two weeks of dosing resulting in a maximal level of 1179±140 nM at time points between 336 and 366 h after initial dosing (mean and SD of data from n=3 animals and 3 time points).

TABLE 26
PK parameters of mAb0705(OA) administered iv in
cynomolgus monkeys at 40 nmol/kg.

| | t_{1/2} | Cl | V_{z} | MRT | AUC |
|---|---|---|---|---|---|
| Animal | (h) | (mL/h × kg) | (mL/kg) | (h) | (h × nM) |
| #1 | 113 | 0.487 | 79.5 | 167 | 82207 |
| #2 | 119 | 0.417 | 71.4 | 175 | 95894 |

Single iv or repeated sc dosing of mAb0705(OA) resulted in accumulation of endogenous FVII(a). Total FVII(a) antigen, FVIIa:AT and FVIIa after single iv dose of 40 nmol/kg are shown in Table 27. The total FVII antigen increased from 7.0 nM pre-dose to 35.0±4.7 nM at day 7-14. Likewise, FVIIa increased from below detection limit (0.009 nM) to 2.3±0.7 nM and FVIIa:AT increased from 1.0 to 6.3±0.8 nM at day 7-14. The zymogen FVII level after administration of 40 nmol/kh one-armed antibody was 26.4 nM as calculated by subtracting FVIIa and FVIIa:AT from total FVII(a) antigen.

TABLE 27
Levels of FVII(a) after a single iv dose of 40 nmol/kg mAb0705(OA)
measured at day 7-14 after dosing.

| | Pre-dose | Steady state |
|---|---|---|
| FVII(a) | level (nM) | concentration (nM) |
| Total FVII(a) antigen | 7.0 | 35.0 ± 4.7 |
| FVIIa:AT | 1.0 | 6.3 ± 0.8 |
| FVIIa | <0.009 | 2.3 ± 0.7 |

The steady state level of total FVII(a), FVIIa and FVIIa:AT after multiple sc dosing are shown in table 28. The total FVII(a) antigen increased from 6.5±1.5 nM pre-dose to 36.9±9.8 nM at day 12-21. Likewise, FVIIa increased from below detection limit (0.009 nM) to 3.9±1.6 nM at day 12-21 and FVIIa:AT increased from 1.0±0.3 to 9.1±0.6 nM at day 12-21. The steady state zymogen FVII level calculated by subtracting FVIIa and FVIIa:AT from total FVII(a) antigen was 24 nM.

The data demonstrates that administration of the one-armed anti-FVII(a) antibody mAb0705(OA) resulted in accumulation of endogenous FVII(a), FVIIa and FVIIa:AT in vivo. The clearance of the one-armed anti-FVII(a) antibody (0.42-0.49 mL/kg×kg, Table 26) was comparable with the clearance of the biAb0001 after i.v. administration (0.33-0.64 mL/kg'kg, Example 27, Table 24). Therefore, the steady-state levels of FVII(a) antigen, FVIIa and FVIIa:AT measured after repeated administration of the one-armed anti-FVII(a) antibody is expected to be representative of the levels that would be achieved at steady state after repeated administration of the biAb with the same FVII(a) binding arm.

TABLE 28
Steady state levels of FVII(a) after repeated sc dosing of 20 nmol/kg
mAb0705(OA) measured at day 12-21 after initial dosing

| | Pre-dose | Steady state |
|---|---|---|
| FVII(a) | level (nM) | concentration (nM) |
| Total FVII(a) antigen | 6.5 ± 1.5 | 36.9 ± 9.8 |
| FVIIa:AT | 1.0 ± 0.3 | 9.1 ± 0.6 |
| FVIIa | <0.009 | 3.9 ± 1.6 |

### Example 29: Thromboelastography at Haemophilia A-Like Conditions in Human Whole Blood Added Anti-FVII(a)/Anti-TLT-1 biAb0001 and Steady State Levels of Zymogen FVII, FVIIa and FVIIa:AT

The effect of the bispecific anti-FVII(a)/anti-TLT-1 antibody biAb0001 (where the parental anti-FVII(a) is mAb0865 and the parental anti-TLT-1 antibody is mAb1076) and accumulated levels of zymogen FVII, FVIIa and FVIIa:AT from Example 28 were evaluated by thromboelastography in human whole blood at haemophilia A-like conditions and compared with the effect of rFVIIa added to the blood. The thromboelastography analysis was carried out using TEG® instruments (Thrombelastograph Coagulation Analyzer, Haemoscope Corp.) in principle as described in Viuff D, et al. Thromb Res 2010; 126:144-9. Citrate-stabilised whole blood from healthy donors was incubated 30 min with 0.1 mg/ml neutralising anti-FVIII sheep polyclonal antibody (Haematological Technologies Inc, cat no PAHFVIII-S-C) and 5 μg/mL neutralising anti-TF mouse monoclonal antibody (1F44, prepared in house). biAb0001 at a final plasma concentration of 100 nM in HBS/BSA buffer (20 mM Hepes, 140 mM NaCl, pH 7,4, 2% BSA) was mixed with rFVIIa (Novo Seven®, Novo Nordisk, final plasma concentration 3.9 nM), zymogen FVII (as prepared in Example 26), final plasma concentration 24 nM) and FVIIa:AT complexes (as prepared in Example 26), final plasma concentration 9 nM) and added to a sample of the blood. Predilutions of FVIIa:AT were made in cold 20 mM MES, 100 mM NaCl, 1 mM EDTA, PH 5.5+2% BSA, and added to the remaining proteins immediately prior to initiating the assay. The FVII zymogen and FVIIa:AT preparations contained trace amounts of FVIIa, hence corresponding lower amount of FVIIa was added to compensate for this and an expected plasma concentration of FVIIa in the donor blood (0.1 nM, Morissey JH et al Blood, 1993; 81:734-44). Likewise was the amount of added zymogen FVII compensated for an expected plasma concentration of 10 nM zymogen FVIIa in the donor blood. A separate sample contained 25 nM rFVIIa corresponding to the theoretical maximal plasma concentration after administration of 90 μg/kg rFVIIa (NovoSeven®) to human subjects with haemophilia A (Lindley C M et al. Clin Pharmacol Ther 1994; 55:638-48). Controls included biAb alone and FVII/FVIIa/FVIIa:AT mixture without biAb. Platelets were maximally activated by adding PAR1 agonist peptide SFLLRN (Tocris Biosciences cat no 3497) to a final concentration of 30 μM and GPVI agonist convulxin (5-Diagnostics, cat no 5D-1192-50UG) to a final concentration of 10 ng/ml. A volume of 20 μL 0.2 M CaCl2 in 20 mM Hepes, pH 7.4, was added to the TEG cup followed by 340 μL of blood samples and the analysis immediately initiated. Clot time (R-time) defined as time to 2 mm amplitude of the TEG-trace was calculated by the software (TEG® Analytical Software, version 4.1.73). Data from 4 donors are shown in Table 29. The clot time was delayed from 290±12 s in normal blood to 3506±1561 s after inducing haemophilia A-like conditions by neutralising FVIII. Adding 25 nM rFVIIa resulted in shortening of the clot time of the mimicked haemophilia A blood to 694±158 s. Adding 100 nM biAb to the blood resulted in a modest reduction of clot time to 2198±712 s, most likely due to a potentiation of the effect of the endogenous FVIIa in the blood. Adding steady state levels of FVII/FVIIa/FVIIa:AT reduced the clot time to 1440±275 s. Combining 100 nM biAb and steady state levels of FVII/FVIIa/FVIIa:AT reduced the clot time to 495±39 s, i.e. to a level comparable to or slightly lower than the clot time after adding 25 nM FVIIa. The data demonstrates that the biAb potentiate the effect of the accumulated level of FVII/FVIIa/FVIIa:AT resulting in a clot time reduction similar to or slightly better than the clot time reduction achieved with a therapeutic effective concentration of rFVIIa.

TABLE 29
Thromboelastography analysis of the effect of biAb0001 (100 nM)
in human steady state levels of FVII/FVIIa/FVIIa:AT.
whole blood with or without

| | Clot time (R-time, s) | | | | | |
|---|---|---|---|---|---|---|
| | | | | | HA + | HA + |
| | | Haemo- | HA + | | FVII/ | biab + FVII/ |
| Date of | | philia A | FVIIa at | HA + | FVIIa/ | FVIIa/ |
| experiment | Normal | (HA) | 25 nM | BiAb | FVIIa:AT | FVIIa:AT |
| 2018 Mar. 14 | 295 | 3160 | 615 | 2460 | 1690 | 445 |
| 2018 Mar. 15 | 280 | 1660 | 540 | 1145 | 1065 | 495 |
| 2018 Mar. 22 | 280 | 3770 | 715 | 2720 | 1415 | 500 |
| 2018 Mar. 21 | 305 | 5433 | 905 | 2465 | 1590 | 540 |
| mean | 290 | 3506 | 694 | 2198 | 1440 | 495 |
| SD | 12 | 1561 | 158 | 712 | 275 | 39 |

### Example 30: Thromboelastography at Haemophilia A-Like Conditions in Human Whole Blood Added Bispecific Anti-FVII(a)/Anti-TLT-1 Antibodies With Different TLT-1 Affinities and Steady State Levels of Zymogen FVII, FVIIa and FVIIa:AT

In the present example the following bispecific anti-FVII(a)/anti-TLT-1 antibodies with different affinities for TLT-1 were tested:

| biAb | Anti-FVII(a) | Anti-TLT-1 | K_{D} (TLT-1) [nM] |
|---|---|---|---|
| biAb0001 | mAb0865 | mAb1076 | 2.9 |
| biAb0015 | mAb0865 | mAb1038 | 19 |
| biAb0090 | mAb0865 | mAb1049 | 320 |
| biAb0095 | mAb0865 | mAb1047 | 75 |

Bispecific antibodies at a concentration of 100 nM were evaluated by thrombo-elastography as described in Example 29. Clot times (R-time) are listed in Table 30. The clot time was delayed from 340 s to 5433 s upon induction of haemophilia A (HA) by adding anti-FVIII antibodies. The presence of the biAb with for the highest affinity for TLT-1 (biAb0001) reduced the clot time to 2465 s i.e. more than the clot time reduction observed for the three other biAbs alone: biAb0015 reduced the clot time to 3645 s; −biAb0090 to 4335 s and biAb0095 to 4110 s. Likewise, combination of biAb with steady state levels of FVII, FVIIa, and FVII:AT resulted in a more pronounced reduction of the clot time for biAb0001 (to 540 s) than for the three other biAbs; i.e. to 1100 s for biAb0015, to 1040 s for biAb0090 and to 815 s for biAb0095 The data demonstrate that biAb0001 with the highest affinity (i.e. lowest KD) for TLT-1 was the most effective in clot time reduction.

TABLE 30
Thromboelastography analysis of the effect of biAb0001,
biAb0015 or biAb0090 (each 100 nM) in human whole blood
with or without steady state levels of FVII/FVIIa/FVIIa:AT.

| Sample | Clot time (R-time, s) |
|---|---|
| Normal | 340 |
| Haemophilia A (HA) | 5433 |
| Haemophilia A (HA) + 25 nM FVIIa | 905 |
| HA + FVII/FVIIa/FVIIa:AT | 1590 |
| HA + biAb0001 | 2465 |
| HA + FVII/FVIIa/FVIIa:AT + biAb0001 | 540 |
| HA + biAb0015 | 3645 |
| HA + FVII/FVIIa/FVIIa:AT + biAb0015 | 1100 |
| HA + biAb0090 | 4335 |
| HA + FVII/FVIIa/FVIIa:AT + biAb0090 | 1040 |
| HA + biAb0095 | 4110 |
| HA + FVII/FVIIa/FVIIa:AT + biAb0095 | 815 |

### Example 31: In Vivo Effect of Anti-FVII(a)/Anti-TLT-1 Bispecific Antibody biAb0001 in Tail Vein Transection Model in FVIII-Deficient Transgenic Human TLT-1 Mice

In vivo efficacy of the anti-FVII(a)/anti-TLT-1 bispecific antibody biAb0001 was determined using a Tail Vein Transection (TVT) model in transgenic FVIII knock-out (i.e. haemophilia A), murine TLT-1 knock-out, human knock-in mice. Since the anti-FVII(a) does not recognize murine FVII(a), the biAb was co-administered with human FVIIa, FVII, FVII:AT to give target plasma levels of these components in the mouse (3.8, 26.2 and 9.0 nM, respectively) mimicking their expected clinical steady-state plasma levels according to Example 28. The concentration of biAb was either 40 or 100 nM. In short, the mice were anaesthetized with isoflurane and placed on a heating pad, set to keep animal body temperature at 37° C., with their tails immersed in saline (37° C.). Dosing was performed in the right lateral tail vein 5 minutes prior to the injury. In the present TVT model (Johansen et al., Haemophilia, 2016, 625-31) the lateral vein was transected. If the bleeding stopped at 10, 20, or 30 min, the tail was taken up from the saline, and the wound was gently wiped with a saline wetted gauze swab. Total blood loss was determined after 40 min by quantifying the amount of haemoglobin in the saline. At 40 min after administration, a blood sample was collected in 3.8% trisodium citrate from the orbital plexus. The blood was centrifuged 5 min at 4000 g and plasma aliquoted and stored at −80° C. until analysis for hIgG (see example 23), total FVII(a) antigen (see example 24), and FVIIa activity (see example 8).

As shown in Table 31, all combinations of FVIIa and biAb led to a significant reduction in blood loss compared with biAb alone or FVII(a) dosed without biAb. For all combinations, platelet count measured 45 min after treatment was comparable to that observed for a vehicle group.

In conclusion, these data demonstrate a significant in vivo haemostatic effect of biAb0001 in the presence of expected steady-state levels of FVIIa, FVII, and FVII:AT.

TABLE 31
Blood loss following tail-vein transection (TVT) in FVIII knock-out/murine TLT-
1 knock-out/human TLT-1 knock-in mice administered combinations of biAb0001, FVIIa,
FVII, and FVIIa:AT as indicated. Administered doses, expected and measured plasma
concentrations, and determined blood loss are shown as mean ± SEM (n = 10). Using a
One-Way ANOVA followed by a Dunnett's multiple comparison test, the blood loss of
group 3-5 were found to be significantly different from group 1 and 2. Total FVII(a)
antigen concentrations (measured according to Example 23) are listed in the rows
labelled ‘FVII’ and values are marked by asterisks.

| | | | Expected | Measured | Blood loss |
|---|---|---|---|---|---|
| | Co-adm. | Dose | plasma | plasma | (nmol |
| Group | compound | (nmol/kg) | conc (nM) | conc (nM) | haem.) |
| 1 | biAb0001 | 0.5 | 4 | 7.1 ± 0.4 | 3935 ± 711 |
| | FVIIa | | | | |
| | FVII | | | | |
| | FVIIa:AT | | | | |
| 2 | biAb0001 | | | | 3225 ± 731 |
| | FVIIa | 0.5 | 3.8 | 1.8 ± 0.2 | |
| | FVII | 3.45 | 26.2 | 37.2 ± 1.9* | |
| | FVIIa:AT | 1.18 | 9 | | |
| 3 | biAb0001 | 0.5 | 4 | 5.5 ± 0.6 | 820 ± 187 |
| | FVIIa | 0.5 | 3.8 | 3.9 ± 0.1 | |
| | FVII | | | 3.8 ± 0.2* | |
| | FVIIa:AT | | | | |
| 4 | biAb0001 | 5.13 | 40 | 126.3 ± 11.1 | 381 ± 63 |
| | FVIIa | 0.5 | 3.8 | 6.3 ± 0.5 | |
| | FVII | 3.45 | 26.2 | 72.4 ± 5.5* | |
| | FVIIa:AT | 1.18 | 9 | | |
| 5 | biAb0001 | 12.83 | 100 | 586.0 ± 115.9 | 598 ± 150 |
| | FVIIa | 0.5 | 3.8 | 5.9 ± 0.4 | |
| | FVII | 3.45 | 26.2 | 124 ± 14.2* | |
| | FVIIa:AT | 1.18 | 9 | | |

While certain features of the invention have been illustrated and described herein, many modifications, substitutions, changes and equivalents will now occur to those of ordinary skill in the art. It is, therefore, to be understood that the appended claims are intended to cover all such modifications and changes as fall within the true spirit of the invention.

### Example 32: Identification of Antibodies Competing With Anti-TLT-1 mAb1076 for Binding to TLT-1 in a Competitive ELISA

The Fab fragment (anti-TLT-1 Fab) to be used for competition experiments comprises the VH and VL domain sequences corresponding to mAb1076 (SEQ ID NO: 938 and 934, respectively), a human IgG4 CH1 domain, and a human kappa CL with a single point mutation (G157C). The G to C substitution is in the constant domain of the Fab fragment, i.e. far from the antigen binding site, and has no influence on the binding to TLT-1 (see Example 20). Recombinant is produced as TLT-1 as described in WO2011/023785. The anti-TLT-1 Fab is biotinylated using standard methods including application of the biotinylation kit (EZ-link, Thermo) according to manufacturer's instructions.

To determine if anti-TLT-1 antibodies compete with the anti-TLT-1 Fab, and antibodies derived thereof, for binding to TLT-1 competition studies were performed. Recombinant TLT-1 is immobilised in a NUNC maxisorp plate over night at 4° C. in dilution buffer (20 mM HEPES, 5 mM CaCl2, 150 mM NaCl, pH 7.2). The plates are washed and blocked in washing buffer (20 mM HEPES, 5 mM CaCl2, 150 mM NaCl, 0.5 mL/L Tween 20, pH 7.2) for 15 min. For the competition study, biotinylated anti-TLT-1 Fab at a final fixed concentration is combined with a dilution series of anti-TLT-1 antibody to give final concentrations ranging from 0 and up to 100 mg/ml in dilution buffer. The mixture is added to the wells of the plate and allowed to incubate for 1 hour. The plate is then washed and HRP-labelled streptavidin-HRPO (1:2000 in dilution buffer; Kirkegaard & Perry Labs) is added and incubated for 1h. Finally, the plate is washed and developed with TMB ONE (KEMENTEC) for 10 min. The reaction is stopped by adding H3PO4 (4M) and the plate read in a FLUOStar Optima plate reader at 450 nm with subtraction of the background signal measured at 620 nm. Unless otherwise specified, all incubations are done at room temperature and plates are washed 5 times using washing buffer.

The concentration of recombinant TLT-1 to be immobilized in the NUNC maxisorp plate as well as the fixed concentration of biotinylated anti-TLT-1 Fab to be mixed with competitor antibody for the competition study are determined by individual titrations of the two components with the aim to give a sufficient signal to allow for detection of competition (i.e. decrease in signal) by competitor antibodies. The concentration of TLT-1 for immobilization is typically in the range from 0-1 mg/ml, such as 125 ng/ml. The concentration of biotinylated anti-TLT-1 Fab is typically in the range from 0-1 mg/ml, such as 10 ng/ml.

From the measured signals (OD units), competition at any given antibody concentration is calculated as

$$\% inhibition = ( 1 - ( OD units - 100 \% inhibition ) / ( 0 \% inhibition - 100 \% inhibition ) * 100$$

where 0% inhibition is determined from the signal in wells without any competing anti-TLT-1 antibody, and 100% inhibition is determined as the signal from wells without biotinylated anti-TLT-1 Fab (i.e. corresponding to the assay background). Antibodies are considered to compete with anti-TLT-1 Fab for binding to TLT-1, if there is at least 50% observed inhibition (% inhibition) when the concentration of the antibody is tested in up to 10000-fold excess of the biotinylated anti-TLT-1 Fab.

1. A bispecific antibody comprising
(i) a first antigen binding site capable of binding Factor VII(a), and
(ii) a second antigen binding site capable of binding TREM-like Transcript 1 (TLT-1).

2. The bispecific antibody according to claim 1, wherein the antibody comprises an Fc region.

3. The bispecific antibody according to claim 1 or 2, wherein the first antigen-binding site competes for binding to FVII(a) with any one of the anti-FVII(a) antibodies comprising a light chain variable domain (VL) and a heavy chain variable domain (VH) as indicated below:
mAb0522 (VL: SEQ ID NO 846 and VH: SEQ ID NO: 850),
Fab0883 (VL: SEQ ID NO 814 and VH: SEQ ID NO: 818),
mAb0005 (VL: SEQ ID NO 750 and VH: SEQ ID NO: 754),
mAb0004 (VL: SEQ ID NO 14 and VH: SEQ ID NO: 18),
mAb0013 (VL: SEQ ID NO 46 and VH: SEQ ID NO: 50),
mAb0018 (VL: SEQ ID NO 62 and VH: SEQ ID NO: 66),
mAb0544 (VL: SEQ ID NO 694 and VH: SEQ ID NO: 698),
mAb0552 (VL: SEQ ID NO 702 and VH: SEQ ID NO: 706),
mAb0001 (VL: SEQ ID NO 710 and VH: SEQ ID NO: 714),
mAb0007 (VL: SEQ ID NO 718 and VH: SEQ ID NO: 722),
mAb0578 (VL: SEQ ID NO 726 and VH: SEQ ID NO: 730),
mAb0701 (VL: SEQ ID NO 734 and VH: SEQ ID NO: 738), and
mAb0587 (VL: SEQ ID NO 742 and VH: SEQ ID NO: 746).

4. The bispecific antibody according to claim 1 or 2, wherein the first antigen-binding site is capable of binding an epitope comprising amino acid residues H115, T130, V131, and R392 of FVII(a) (SEQ ID NO: 1).

5. The bispecific antibody according to claim 1 or 2, wherein the first antigen-binding site is capable of binding an epitope comprising the following amino acid residues R113, C114, H115, E116, G117, Y118, S119, L120, T130, V131, N184, T185, P251, V252, V253, Q388, M391 and R392 of FVII(a) (SEQ ID NO: 1).

6. The bispecific antibody according to claim 1 or 2, wherein the first antigen-binding site comprises:
the CDRL1 represented by amino acid residues RASQGISDYLH (SEQ ID NO: 847), with 0, 1, 2 or 3 amino acid substitutions
the CDRL2 represented by amino acid residues YTSQPAT (SEQ ID NO: 848), with 0, 1, 2 or 3 amino acid substitutions
the CDRL3 represented by amino acid residues QNGHSFPLT (SEQ ID NO: 849), with 0, 1 or 2 amino acid substitutions
the CDRH1 represented by amino acid residues SDSAWS (SEQ ID NO: 851),
the CDRH2 represented by amino acid residues YIQYSGST NYNPSLKS (SEQ ID NO: 852), with 0 or 1 amino acid substitutions
the CDRH3 represented by amino acid residues SVNYYGNSFAVGY (SEQ ID NO: 853), with 0, 1, 2 and 3 amino acid substitutions.

7. The bispecific antibody according to claim 1 or 2, wherein the first antigen-binding site comprises a light chain variable domain identified by SEQ ID NO:846 and a heavy chain variable domain identified by SEQ ID NO:850.

8. The bispecific antibody according to claim 1 or 2, wherein the second antigen-binding site competes for binding to TLT-1 with any one of the anti-TLT-1 antibodies comprising a light chain variable domain (VL) and a heavy chain variable domain (VH) as indicated below:
mAb0524 (VL: SEQ ID NO 854 and VH: SEQ ID NO: 858),
mAb0012 (VL: SEQ ID NO 862 and VH: SEQ ID NO: 866),
mAb0023 (VL: SEQ ID NO 870 and VH: SEQ ID NO: 874),
mAb0051 (VL: SEQ ID NO 878 and VH: SEQ ID NO: 882), and
mAb0062 (VL: SEQ ID NO 894 and VH: SEQ ID NO: 898).

9. The bispecific antibody according to claim 1 or 2, wherein the second antigen-binding site is capable of binding an epitope comprising the following amino acid residues K8, I9, G10, S11, L12, A13, N15, A16, F17, S18, D19, P20, A21 of TLT-1 (SEQ ID NO: 13).

10. The bispecific antibody according to claim 1 or 2, wherein the second antigen-binding site comprises:
the CDRL1 represented by SEQ ID NO: 855,
the CDRL2 represented by SEQ ID NO: 856,
the CDRL3 represented by SEQ ID NO: 857,
the CDRH1 represented by SEQ ID NO: 859,
the CDRH2 represented by SEQ ID NO: 860,
the CDRH3 represented by SEQ ID NO: 861.

11. The bispecific antibody according to claim 1 or 2, wherein the second antigen-binding site is comprised by a light chain variable domain identified by SEQ ID NO:854 and a heavy chain variable domain identified by SEQ ID NO:858.

12. The bispecific antibody according to claim 1, wherein the first antigen-binding site comprises a light chain variable domain identified by SEQ ID NO:846 and a heavy chain variable domain identified by SEQ ID NO:850, and wherein the second antigen-binding site is comprised by a light chain variable domain identified by SEQ ID NO: 854 and a heavy chain variable domain identified by SEQ ID NO:858.

13. The bispecific antibody according to claim 1, wherein the first antigen-binding site comprises a first light chain variable domain identified by SEQ ID NO:846 and a first heavy chain variable domain identified by SEQ ID NO:850, and wherein the second antigen-binding site is comprised by a second light chain variable domain identified by SEQ ID NO: 854 and a second heavy chain variable domain identified by SEQ ID NO:858 and wherein the heavy chain constant domains attached to first and second heavy chain variable domain are identified by SEQ ID NO: 943 and 942, respectively and wherein the light chain constant domains attached to first and second light chain variable domain are both identified by SEQ ID NO: 12.

14. A pharmaceutical formulation comprising the bispecific antibody according to any one of the preceding claims and a pharmaceutically acceptable carrier.

15. The bispecific antibody according to any one of claims 1-13 or the pharmaceutical formulation according to claim 14 for use in the treatment of a coagulopathy, wherein said coagulopathy is selected from the group consisting of haemophilia A, with or without inhibitors or haemophilia B, with or without inhibitors, FVII(a) deficiency and Glanzmann's thrombasthenia.


SEQUENCE LISTING (945 sequences)

SEQ ID NO: 1 - protein, 406 aa, synthetic construct [sequence omitted]

SEQ ID NO: 2 - protein, 296 aa, Homo sapiens
QGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIPLIWGAVLLVGLLVAAVVLFAVMAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS

SEQ ID NO: 3 - protein, 147 aa, Homo sapiens
QGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIP

SEQ ID NO: 4 - protein, 327 aa, synthetic construct
  REGION 1..327: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 327 aa, synthetic construct
  REGION 1..327: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 6 - protein, 327 aa, synthetic construct
  REGION 1..327: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 327 aa, synthetic construct
  REGION 1..327: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 8 - protein, 230 aa, synthetic construct
  REGION 1..230: note = Recombinant protein
VESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 9 - protein, 327 aa, synthetic construct
  REGION 1..327: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 10 - protein, 229 aa, synthetic construct
  REGION 1..229: note = Recombinant protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 98 aa, synthetic construct
  REGION 1..98: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 12 - protein, 107 aa, synthetic construct [sequence omitted]

SEQ ID NO: 13 - protein, 37 aa, synthetic construct
  REGION 1..37: note = Recombinant protein
EEEEETHKIGSLAENAFSDPAGSANPLEPSQDEKSIP

SEQ ID NO: 14 - protein, 107 aa, Mus musculus
DIVMTQSHKIMSTLVGDRVSITCKASQDVDPAVAWYQQKPGQYPKLLIYWASTRHTGVPDRFTGSGSGTDFTLTISNVQSEDLADYFCQQYSKYPYTFGGGTKLEIK

SEQ ID NO: 15 - protein, 11 aa, Mus musculus
KASQDVDPAVA

SEQ ID NO: 16 - protein, 7 aa, Mus musculus
WASTRHT

SEQ ID NO: 17 - protein, 9 aa, Mus musculus
QQYSKYPYT

SEQ ID NO: 18 - protein, 118 aa, Mus musculus
QVQLKESGPGLVAPSQSLSITCTVSGFSLTDYGVSWIRQPPGKGLEWLGVIWGGGNTYNNSALKSRLSISKDNSKSQVFLKMNSLQADDTAMYFCAKHGYYGYVMDYWGQGTSVTVSS

SEQ ID NO: 19 - protein, 5 aa, Mus musculus
DYGVS

SEQ ID NO: 20 - protein, 16 aa, Mus musculus
VIWGGGNTYNNSALKS

SEQ ID NO: 21 - protein, 10 aa, Mus musculus
HGYYGYVMDY

SEQ ID NO: 22 - protein, 107 aa, synthetic construct
  REGION 1..107: note = Recombinant protein
DIVMTQSPATLSVTAGDRVSLSCRASQGISDYLHWYQQKSHESPRLLIKYTSQPVSGIPSRFSGSGSGSEFTLSINSVEPEDVGVYYCQNGHSFPLTFGAGTKLELK

SEQ ID NO: 23 - protein, 11 aa, synthetic construct
  REGION 1..11: note = Recombinant protein
RASQGISDYLH

SEQ ID NO: 24 - protein, 7 aa, synthetic construct
  REGION 1..7: note = Recombinant protein
YTSQPVS

SEQ ID NO: 25 - protein, 9 aa, synthetic construct
  REGION 1..9: note = Recombinant protein
QNGHSFPLT

SEQ ID NO: 26 - protein, 122 aa, synthetic construct
  REGION 1..122: note = Recombinant protein
DVQLQESGPGLVEPSQSLSLTCTVTGYSITSDCAWSWIRQFPGNKLEWMGYINYSGSTNYNPSLKSRISVTRDTSKNQFFLRLSSVTAEDTATYYCTRSVNYYGNSFAVGYWGQGTSVTVSS

SEQ ID NO: 27 - protein, 6 aa, synthetic construct
  REGION 1..6: note = Recombinant protein
SDCAWS

SEQ ID NO: 28 - protein, 16 aa, synthetic construct
  REGION 1..16: note = Recombinant protein
YINYSGSTNYNPSLKS

SEQ ID NO: 29 - protein, 13 aa, synthetic construct
  REGION 1..13: note = Recombinant protein
SVNYYGNSFAVGY

SEQ ID NO: 30 - protein, 107 aa, synthetic construct; identical to SEQ ID NO: 22
  REGION 1..107: note = Recombinant protein

SEQ ID NO: 31 - protein, 11 aa, synthetic construct
  REGION 1..11: note = Recombinant protein
RASQGISDYLH

SEQ ID NO: 32 - protein, 7 aa, synthetic construct
  REGION 1..7: note = Recombinant protein
YTSQPVS

SEQ ID NO: 33 - protein, 9 aa, synthetic construct
  REGION 1..9: note = Recombinant protein
QNGHSFPLT

SEQ ID NO: 34 - protein, 122 aa, synthetic construct; identical to SEQ ID NO: 26
  REGION 1..122: note = Recombinant protein

SEQ ID NO: 35 - protein, 6 aa, synthetic construct
  REGION 1..6: note = Recombinant protein
SDCAWS

SEQ ID NO: 36 - protein, 16 aa, synthetic construct
  REGION 1..16: note = Recombinant protein
YINYSGSTNYNPSLKS

SEQ ID NO: 37 - protein, 13 aa, synthetic construct
  REGION 1..13: note = Recombinant protein
SVNYYGNSFAVGY

SEQ ID NO: 38 - protein, 107 aa, synthetic construct; identical to SEQ ID NO: 22
  REGION 1..107: note = Recombinant protein

SEQ ID NO: 39 - protein, 11 aa, synthetic construct
  REGION 1..11: note = Recombinant protein
RASQGISDYLH

SEQ ID NO: 40 - protein, 7 aa, synthetic construct
  REGION 1..7: note = Recombinant protein
YTSQPVS

SEQ ID NO: 41 - protein, 9 aa, synthetic construct
  REGION 1..9: note = Recombinant protein
QNGHSFPLT

SEQ ID NO: 42 - protein, 122 aa, synthetic construct; identical to SEQ ID NO: 26
  REGION 1..122: note = Recombinant protein

SEQ ID NO: 43 - protein, 6 aa, synthetic construct
  REGION 1..6: note = Recombinant protein
SDCAWS

SEQ ID NO: 44 - protein, 16 aa, synthetic construct
  REGION 1..16: note = Recombinant protein
YINYSGSTNYNPSLKS

SEQ ID NO: 45 - protein, 13 aa, synthetic construct
  REGION 1..13: note = Recombinant protein
SVNYYGNSFAVGY

SEQ ID NO: 46 - protein, 113 aa, Mus musculus
DIVMTQSPSSLTVTAGEKVTVSCKSSQSLLNSGDQKNYLTWYQQKPGQPPKLLIYWASTRESGVPDRFTGSGSGTDFTLTISSVQAEDLAVYYCQNDFSYPYTFGGGTKLEIK

SEQ ID NO: 47 - protein, 17 aa, Mus musculus
KSSQSLLNSGDQKNYLT

SEQ ID NO: 48 - protein, 7 aa, Mus musculus
WASTRES

SEQ ID NO: 49 - protein, 9 aa, Mus musculus
QNDFSYPYT

SEQ ID NO: 50 - protein, 118 aa, Mus musculus
QIQLVQSGPELKKPGETVKISCKASGYTFTDYSIQWVKQAPGKGLKWMGWINTETGKPSYADDLKGRSAFSLETSGSTAYLQISNLKNEDMATYFCVRDDYYGREDYWGQGTTLTVSS

SEQ ID NO: 51 - protein, 5 aa, Mus musculus
DYSIQ

SEQ ID NO: 52 - protein, 17 aa, Mus musculus
WINTETGKPSYADDLKG

SEQ ID NO: 53 - protein, 9 aa, Mus musculus
DDYYGREDY

SEQ ID NO: 54 - protein, 107 aa, synthetic construct; identical to SEQ ID NO: 22
  REGION 1..107: note = Recombinant protein

SEQ ID NO: 55 - protein, 11 aa, synthetic construct
  REGION 1..11: note = Recombinant protein
RASQGISDYLH

SEQ ID NO: 56 - protein, 7 aa, synthetic construct
  REGION 1..7: note = Recombinant protein
YTSQPVS

SEQ ID NO: 57 - protein, 9 aa, synthetic construct
  REGION 1..9: note = Recombinant protein
QNGHSFPLT

SEQ ID NO: 58 - protein, 122 aa, synthetic construct
  REGION 1..122: note = Recombinant protein
DVQLQESGPGLVEPSQSLSLTCTVTGYSITSDSAWSWIRQFPGNKLEWMGYIQYSGSTNYNPSLKSRISVTRDTSKNQFFLRLSSVTAEDTATYYCTRSVNYYGNSFAVGYWGQGTSVTVSS

SEQ ID NO: 59 - protein, 6 aa, synthetic construct
  REGION 1..6: note = Recombinant protein
SDSAWS

SEQ ID NO: 60 - protein, 16 aa, synthetic construct
  REGION 1..16: note = Recombinant protein
YIQYSGSTNYNPSLKS

SEQ ID NO: 61 - protein, 13 aa, synthetic construct
  REGION 1..13: note = Recombinant protein
SVNYYGNSFAVGY

SEQ ID NO: 62 - protein, 113 aa, Mus musculus
DIVLSQSPSSLAVSVGEKVTMSCKSSQSLLYSSNQKNSLAWYQHKPGQSPKLLIYWASTRESGVPDRFTGSGSGTDFTLTISNVKAEDLAVYYCHQYYNYLLTFGPGTKLELK

SEQ ID NO: 63 - protein, 17 aa, Mus musculus
KSSQSLLYSSNQKNSLA

SEQ ID NO: 64 - protein, 7 aa, Mus musculus
WASTRES

SEQ ID NO: 65 - protein, 9 aa, Mus musculus
HQYYNYLLT

SEQ ID NO: 66 - protein, 123 aa, Mus musculus
QITLKESGPGILKPSQTLSLTCSFSGFSLSTSGLGVGWIRQTSGKGLEWLAHIWWDDDKYYYPPLKSQLTISKDTSRNQVFLKITSVDTADTATYYCARRAFYDGYYGPMEYWGQGTSVTVSS

SEQ ID NO: 67 - protein, 7 aa, Mus musculus
TSGLGVG

SEQ ID NO: 68 - protein, 16 aa, Mus musculus
HIWWDDDKYYYPPLKS

SEQ ID NO: 69 - protein, 13 aa, Mus musculus
RAFYDGYYGPMEY

SEQ ID NO: 70 - protein, 107 aa, synthetic construct
  REGION 1..107: note = Recombinant protein
EIVLTQSPATLSLSPGERATLSCRASQGVSSYLHWYQQKPGQAPRLLIKYASQPATGIPARFSGSGSGTDFTLTISSLEPEDFAVYYCQSGHSFPYTFGQGTKLEIK

SEQ ID NO: 71 - protein, 11 aa, synthetic construct
  REGION 1..11: note = Recombinant protein
RASQGVSSYLH

SEQ ID NO: 72 - protein, 7 aa, synthetic construct
  REGION 1..7: note = Recombinant protein
YASQPAT

SEQ ID NO: 73 - protein, 9 aa, synthetic construct
  REGION 1..9: note = Recombinant protein
QSGHSFPYT

SEQ ID NO: 74 - protein, 122 aa, synthetic construct; identical to SEQ ID NO: 58
  REGION 1..122: note = Recombinant protein

SEQ ID NO: 75 - protein, 6 aa, synthetic construct
  REGION 1..6: note = Recombinant protein
SDSAWS

SEQ ID NO: 76 - protein, 16 aa, synthetic construct
  REGION 1..16: note = Recombinant protein
YIQYSGSTNYNPSLKS

SEQ ID NO: 77 - protein, 13 aa, synthetic construct
  REGION 1..13: note = Recombinant protein
SVNYYGNSFAVGY

SEQ ID NO: 78 - protein, 107 aa, synthetic construct
  REGION 1..107: note = Recombinant protein
EIVLTQSPATLSLSPGERATLSCRASQGISDYLHWYQQKPGEAPRLLIKYTSQPATGIPARFSGSGSGTDFTLTISSLEPEDVGVYYCQSGHSFPLTFGAGTKLELK

SEQ ID NO: 79 - protein, 11 aa, synthetic construct
  REGION 1..11: note = Recombinant protein
RASQGISDYLH

SEQ ID NO: 80 - protein, 7 aa, synthetic construct
  REGION 1..7: note = Recombinant protein
YTSQPAT

SEQ ID NO: 81 - protein, 9 aa, synthetic construct
  REGION 1..9: note = Recombinant protein
QSGHSFPLT

SEQ ID NO: 82 - protein, 122 aa, synthetic construct; identical to SEQ ID NO: 58
  REGION 1..122: note = Recombinant protein

SEQ ID NO: 83 - protein, 6 aa, synthetic construct
  REGION 1..6: note = Recombinant protein
SDSAWS

SEQ ID NO: 84 - protein, 16 aa, synthetic construct
  REGION 1..16: note = Recombinant protein
YIQYSGSTNYNPSLKS

SEQ ID NO: 85 - protein, 13 aa, synthetic construct
  REGION 1..13: note = Recombinant protein
SVNYYGNSFAVGY

SEQ ID NO: 86 - protein, 107 aa, synthetic construct; identical to SEQ ID NO: 22
  REGION 1..107: note = Recombinant protein

SEQ ID NO: 87 - protein, 11 aa, synthetic construct
  REGION 1..11: note = Recombinant protein
RASQGISDYLH

SEQ ID NO: 88 - protein, 7 aa, synthetic construct
  REGION 1..7: note = Recombinant protein
YTSQPVS

SEQ ID NO: 89 - protein, 9 aa, synthetic construct
  REGION 1..9: note = Recombinant protein
QNGHSFPLT

SEQ ID NO: 90 - protein, 122 aa, synthetic construct
  REGION 1..122: note = Recombinant protein
DVQLQESGPGLVKPSQTLSLTCTVSGYSISSDSAWSWIRQPPGKGLEWIGYIQYSGSTNYNPSLKSRVTISRDTSKNQFSLKLSSVTAADTAVYYCARSVNYYGNSFWVDPWGQGTLVTVSS

SEQ ID NO: 91 - protein, 6 aa, synthetic construct
  REGION 1..6: note = Recombinant protein
SDSAWS

SEQ ID NO: 92 - protein, 16 aa, synthetic construct
  REGION 1..16: note = Recombinant protein
YIQYSGSTNYNPSLKS

SEQ ID NO: 93 - protein, 13 aa, synthetic construct
  REGION 1..13: note = Recombinant protein
SVNYYGNSFWVDP

SEQ ID NO: 94 - protein, 107 aa, synthetic construct; identical to SEQ ID NO: 70
  REGION 1..107: note = Recombinant protein

SEQ ID NO: 95 - protein, 11 aa, synthetic construct
  REGION 1..11: note = Recombinant protein
RASQGVSSYLH

SEQ ID NO: 96 - protein, 7 aa, synthetic construct
  REGION 1..7: note = Recombinant protein
YASQPAT

SEQ ID NO: 97 - protein, 9 aa, synthetic construct
  REGION 1..9: note = Recombinant protein
QSGHSFPYT

SEQ ID NO: 98 - protein, 122 aa, synthetic construct; identical to SEQ ID NO: 90
  REGION 1..122: note = Recombinant protein

SEQ ID NO: 99 - protein, 6 aa, synthetic construct
  REGION 1..6: note = Recombinant protein
SDSAWS

SEQ ID NO: 100 - protein, 16 aa, synthetic construct
  REGION 1..16: note = Recombinant protein
YIQYSGSTNYNPSLKS

SEQ ID NO: 101 - protein, 13 aa, synthetic construct
  REGION 1..13: note = Recombinant protein
SVNYYGNSFWVDP

SEQ ID NO: 102 - protein, 107 aa, synthetic construct
  REGION 1..107: note = Recombinant protein
EIVLTQSPATLSLSPGERATLSCRASQGISDYLHWYQQKPGEAPRLLIKYTSQPATGIPARFSGSGSGTDFTLTISSLEPEDVGVYYCQNGHSFPLTFGAGTKLELK

SEQ ID NO: 103 - protein, 11 aa, synthetic construct
  REGION 1..11: note = Recombinant protein
RASQGISDYLH

SEQ ID NO: 104 - protein, 7 aa, synthetic construct
  REGION 1..7: note = Recombinant protein
YTSQPAT

SEQ ID NO: 105 - protein, 9 aa, synthetic construct
  REGION 1..9: note = Recombinant protein
QNGHSFPLT

SEQ ID NO: 106 - protein, 122 aa, synthetic construct
  REGION 1..122: note = Recombinant protein
DVQLQESGPGLVEPSQTLSLTCTVSGYSITSDSAWSWIRQFPGKKLEWMGYIQYSGSTNYNPSLKSRVTISRDTSKNQFSLKLSSVTAEDTAVYYCARSVNYYGNSFWVGYWGQGTSVTVSS

SEQ ID NO: 107 - protein, 6 aa, synthetic construct
  REGION 1..6: note = Recombinant protein
SDSAWS

SEQ ID NO: 108 - protein, 16 aa, synthetic construct
  REGION 1..16: note = Recombinant protein
YIQYSGSTNYNPSLKS

SEQ ID NO: 109 - protein, 13 aa, synthetic construct
  REGION 1..13: note = Recombinant protein
SVNYYGNSFWVGY

SEQ ID NO: 110 - protein, 107 aa, synthetic construct; identical to SEQ ID NO: 102
  REGION 1..107: note = Recombinant protein

SEQ ID NO: 111 - protein, 11 aa, synthetic construct
  REGION 1..11: note = Recombinant protein
RASQGISDYLH

SEQ ID NO: 112 - protein, 7 aa, synthetic construct
  REGION 1..7: note = Recombinant protein
YTSQPAT

SEQ ID NO: 113 - protein, 9 aa, synthetic construct
  REGION 1..9: note = Recombinant protein
QNGHSFPLT

SEQ ID NO: 114 - protein, 122 aa, synthetic construct
  REGION 1..122: note = Recombinant protein
DVQLQESGPGLVKPSQTLSLTCTVSGYSITSDSAWSWIRQFPGKKLEWMGYIQYSGSTNYNPSLKSRVTISRDTSKNQFSLKLSSVTAEDTAVYYCARSVNYYGNSFAVGYWGQGTSVTVSS

SEQ ID NO: 115 - protein, 6 aa, synthetic construct
  REGION 1..6: note = Recombinant protein
SDSAWS

SEQ ID NO: 116 - protein, 16 aa, synthetic construct
  REGION 1..16: note = Recombinant protein
YIQYSGSTNYNPSLKS

SEQ ID NO: 117 - protein, 13 aa, synthetic construct
  REGION 1..13: note = Recombinant protein
SVNYYGNSFAVGY

SEQ ID NO: 118 - protein, 107 aa, synthetic construct; identical to SEQ ID NO: 102
  REGION 1..107: note = Recombinant protein

SEQ ID NO: 119 - protein, 11 aa, synthetic construct
  REGION 1..11: note = Recombinant protein
RASQGISDYLH

SEQ ID NO: 120 - protein, 7 aa, synthetic construct
  REGION 1..7: note = Recombinant protein
YTSQPAT

SEQ ID NO: 121 - protein, 9 aa, synthetic construct
  REGION 1..9: note = Recombinant protein
QNGHSFPLT

SEQ ID NO: 122 - protein, 122 aa, synthetic construct
  REGION 1..122: note = Recombinant protein
DVQLQESGPGLVEPSQTLSLTCTVSGYSITSDSAWSWIRQFPGKKLEWMGYIQYSGSTNYNPSLKSRVTISRDTSKNQFSLKLSSVTAADTAVYYCARSVNYYGNSFAVGYWGQGTSVTVSS

SEQ ID NO: 123 - protein, 6 aa, synthetic construct
  REGION 1..6: note = Recombinant protein
SDSAWS

SEQ ID NO: 124 - protein, 16 aa, synthetic construct
  REGION 1..16: note = Recombinant protein
YIQYSGSTNYNPSLKS

SEQ ID NO: 125 - protein, 13 aa, synthetic construct
  REGION 1..13: note = Recombinant protein
SVNYYGNSFAVGY

SEQ ID NO: 126 - protein, 107 aa, synthetic construct; identical to SEQ ID NO: 102
  REGION 1..107: note = Recombinant protein

SEQ ID NO: 127 - protein, 11 aa, synthetic construct
  REGION 1..11: note = Recombinant protein
RASQGISDYLH

SEQ ID NO: 128 - protein, 7 aa, synthetic construct
  REGION 1..7: note = Recombinant protein
YTSQPAT

SEQ ID NO: 129 - protein, 9 aa, synthetic construct
  REGION 1..9: note = Recombinant protein
QNGHSFPLT

SEQ ID NO: 130 - protein, 122 aa, synthetic construct
  REGION 1..122: note = Recombinant protein
DVQLQESGPGLVEPSQTLSLTCTVSGYSITSDSAWSWIRQPPGKKLEWMGYIQYSGSTNYNPSLKSRVTISRDTSKNQFSLKLSSVTAEDTAVYYCARSVNYYGNSFAVGYWGQGTSVTVSS

SEQ ID NO: 131 - protein, 6 aa, synthetic construct
  REGION 1..6: note = Recombinant protein
SDSAWS

SEQ ID NO: 132 - protein, 16 aa, synthetic construct
  REGION 1..16: note = Recombinant protein
YIQYSGSTNYNPSLKS

SEQ ID NO: 133 - protein, 13 aa, synthetic construct
  REGION 1..13: note = Recombinant protein
SVNYYGNSFAVGY

SEQ ID NO: 134 - protein, 107 aa, synthetic construct; identical to SEQ ID NO: 102
  REGION 1..107: note = Recombinant protein

SEQ ID NO: 135 - protein, 11 aa, synthetic construct
  REGION 1..11: note = Recombinant protein
RASQGISDYLH

SEQ ID NO: 136 - protein, 7 aa, synthetic construct
  REGION 1..7: note = Recombinant protein
YTSQPAT

SEQ ID NO: 137 - protein, 9 aa, synthetic construct
  REGION 1..9: note = Recombinant protein
QNGHSFPLT

SEQ ID NO: 138 - protein, 122 aa, synthetic construct
  REGION 1..122: note = Recombinant protein
DVQLQESGPGLVEPSQTLSLTCTVSGYSITSDSAWSWIRQFPGKKLEWMGYIQYSGSTNYNPSLKSRVTISRDTSKNQFSLKLSSVTAEDTAVYYCARSVNYYGNSFAVDYWGQGTSVTVSS

SEQ ID NO: 139 - protein, 6 aa, synthetic construct
  REGION 1..6: note = Recombinant protein
SDSAWS

SEQ ID NO: 140 - protein, 16 aa, synthetic construct
  REGION 1..16: note = Recombinant protein
YIQYSGSTNYNPSLKS

SEQ ID NO: 141 - protein, 13 aa, synthetic construct
  REGION 1..13: note = Recombinant protein
SVNYYGNSFAVDY

SEQ ID NO: 142 - protein, 107 aa, synthetic construct; identical to SEQ ID NO: 102
  REGION 1..107: note = Recombinant protein

SEQ ID NO: 143 - protein, 11 aa, synthetic construct
  REGION 1..11: note = Recombinant protein
RASQGISDYLH

SEQ ID NO: 144 - protein, 7 aa, synthetic construct
  REGION 1..7: note = Recombinant protein
YTSQPAT

SEQ ID NO: 145 - protein, 9 aa, synthetic construct
  REGION 1..9: note = Recombinant protein
QNGHSFPLT

SEQ ID NO: 146 - protein, 122 aa, synthetic construct
  REGION 1..122: note = Recombinant protein
DVQLQESGPGLVEPSQTLSLTCTVSGYSITSDSAWSWIRQFPGKGLEWMGYIQYSGSTNYNPSLKSRVTISRDTSKNQFSLKLSSVTAEDTAVYYCARSVNYYGNSFAVGYWGQGTSVTVSS

SEQ ID NO: 147 - protein, 6 aa, synthetic construct
  REGION 1..6: note = Recombinant protein
SDSAWS

SEQ ID NO: 148 - protein, 16 aa, synthetic construct
  REGION 1..16: note = Recombinant protein
YIQYSGSTNYNPSLKS

SEQ ID NO: 149 - protein, 13 aa, synthetic construct
  REGION 1..13: note = Recombinant protein
SVNYYGNSFAVGY

SEQ ID NO: 150 - protein, 107 aa, synthetic construct; identical to SEQ ID NO: 102
  REGION 1..107: note = Recombinant protein

SEQ ID NO: 151 - protein, 11 aa, synthetic construct
  REGION 1..11: note = Recombinant protein
RASQGISDYLH

SEQ ID NO: 152 - protein, 7 aa, synthetic construct
  REGION 1..7: note = Recombinant protein
YTSQPAT

SEQ ID NO: 153 - protein, 9 aa, synthetic construct
  REGION 1..9: note = Recombinant protein
QNGHSFPLT

SEQ ID NO: 154 - protein, 122 aa, synthetic construct
  REGION 1..122: note = Recombinant protein
DVQLQESGPGLVEPSQTLSLTCTVSGYSITSDSAWSWIRQFPGKKLEWIGYIQYSGSTNYNPSLKSRVTISRDTSKNQFSLKLSSVTAEDTAVYYCARSVNYYGNSFAVGYWGQGTSVTVSS

SEQ ID NO: 155 - protein, 6 aa, synthetic construct
  REGION 1..6: note = Recombinant protein
SDSAWS

SEQ ID NO: 156 - protein, 16 aa, synthetic construct
  REGION 1..16: note = Recombinant protein
YIQYSGSTNYNPSLKS

SEQ ID NO: 157 - protein, 13 aa, synthetic construct
  REGION 1..13: note = Recombinant protein
SVNYYGNSFAVGY

SEQ ID NO: 158 - protein, 107 aa, synthetic construct; identical to SEQ ID NO: 102
  REGION 1..107: note = Recombinant protein

SEQ ID NO: 159 - protein, 11 aa, synthetic construct
  REGION 1..11: note = Recombinant protein
RASQGISDYLH

SEQ ID NO: 160 - protein, 7 aa, synthetic construct
  REGION 1..7: note = Recombinant protein
YTSQPAT

SEQ ID NO: 161 - protein, 9 aa, synthetic construct
  REGION 1..9: note = Recombinant protein
QNGHSFPLT

SEQ ID NO: 162 - protein, 122 aa, synthetic construct
  REGION 1..122: note = Recombinant protein
DVQLQESGPGLVEPSQTLSLTCTVSGYSITSDSAWSWIRQFPGKKLEWMGYIQYSGSTNYNPSLKSRVTISRDTSKNQFSLKLSSVTAEDTAVYYCARSVNYYGNSFAVGYWGQGTLVTVSS

SEQ ID NO: 163 - protein, 6 aa, synthetic construct
  REGION 1..6: note = Recombinant protein
SDSAWS

SEQ ID NO: 164 - protein, 16 aa, synthetic construct
  REGION 1..16: note = Recombinant protein
YIQYSGSTNYNPSLKS

SEQ ID NO: 165 - protein, 13 aa, synthetic construct
  REGION 1..13: note = Recombinant protein
SVNYYGNSFAVGY

SEQ ID NO: 166 - protein, 107 aa, synthetic construct; identical to SEQ ID NO: 102
  REGION 1..107: note = Recombinant protein

SEQ ID NO: 167 - protein, 11 aa, synthetic construct
  REGION 1..11: note = Recombinant protein
RASQGISDYLH

SEQ ID NO: 168 - protein, 7 aa, synthetic construct
  REGION 1..7: note = Recombinant protein
YTSQPAT

SEQ ID NO: 169 - protein, 9 aa, synthetic construct
  REGION 1..9: note = Recombinant protein
QNGHSFPLT

SEQ ID NO: 170 - protein, 122 aa, synthetic construct
  REGION 1..122: note = Recombinant protein
DVQLQESGPGLVEPSQTLSLTCTVSGYSISSDSAWSWIRQFPGKKLEWMGYIQYSGSTNYNPSLKSRVTISRDTSKNQFSLKLSSVTAEDTAVYYCARSVNYYGNSFAVGYWGQGTSVTVSS

SEQ ID NO: 171 - protein, 6 aa, synthetic construct
  REGION 1..6: note = Recombinant protein
SDSAWS

SEQ ID NO: 172 - protein, 16 aa, synthetic construct
  REGION 1..16: note = Recombinant protein
YIQYSGSTNYNPSLKS

SEQ ID NO: 173 - protein, 13 aa, synthetic construct
  REGION 1..13: note = Recombinant protein
SVNYYGNSFAVGY

SEQ ID NO: 174 - protein, 107 aa, synthetic construct; identical to SEQ ID NO: 102
  REGION 1..107: note = Recombinant protein

SEQ ID NO: 175 - protein, 11 aa, synthetic construct
  REGION 1..11: note = Recombinant protein
RASQGISDYLH

SEQ ID NO: 176 - protein, 7 aa, synthetic construct
  REGION 1..7: note = Recombinant protein
YTSQPAT

SEQ ID NO: 177 - protein, 9 aa, synthetic construct
  REGION 1..9: note = Recombinant protein
QNGHSFPLT

SEQ ID NO: 178 - protein, 122 aa, synthetic construct
  REGION 1..122: note = Recombinant protein
DVQLQESGPGLVEPSQTLSLTCTVSGYSITSDSAWSWIRQFPGKKLEWMGYIQYSGSTNYNPSLKSRVTISRDTSKNQFSLKLSSVTAEDTAVYYCARSVNYYGNSFAVGPWGQGTSVTVSS

SEQ ID NO: 179 - protein, 6 aa, synthetic construct
  REGION 1..6: note = Recombinant protein
SDSAWS

SEQ ID NO: 180 - protein, 16 aa, synthetic construct
  REGION 1..16: note = Recombinant protein
YIQYSGSTNYNPSLKS

SEQ ID NO: 181 - protein, 13 aa, synthetic construct
  REGION 1..13: note = Recombinant protein
SVNYYGNSFAVGP

SEQ ID NO: 182 - protein, 107 aa, synthetic construct
  REGION 1..107: note = Recombinant protein
EIVLTQSPATLSLSPGERATLSCRASQGISDYLHWYQQKPGEAPRLLIKYTSQPATGIPARFSGSGSGTDFTLTISSLEPEDVGVYYCQNGHSFPLTFGQGTKLELK

SEQ ID NO: 183 - protein, 11 aa, synthetic construct
  REGION 1..11: note = Recombinant protein
RASQGISDYLH

SEQ ID NO: 184 - protein, 7 aa, synthetic construct
  REGION 1..7: note = Recombinant protein
YTSQPAT

SEQ ID NO: 185 - protein, 9 aa, synthetic construct
  REGION 1..9: note = Recombinant protein
QNGHSFPLT

SEQ ID NO: 186 - protein, 122 aa, synthetic construct
  REGION 1..122: note = Recombinant protein
DVQLQESGPGLVEPSQTLSLTCTVSGYSITSDSAWSWIRQFPGKKLEWMGYIQYSGSTNYNPSLKSRVTISRDTSKNQFSLKLSSVTAEDTAVYYCARSVNYYGNSFAVGYWGQGTSVTVSS

SEQ ID NO: 187 - protein, 6 aa, synthetic construct
  REGION 1..6: note = Recombinant protein
SDSAWS

SEQ ID NO: 188 - protein, 16 aa, synthetic construct
  REGION 1..16: note = Recombinant protein
YIQYSGSTNYNPSLKS

SEQ ID NO: 189 - protein, 13 aa, synthetic construct
  REGION 1..13: note = Recombinant protein
SVNYYGNSFAVGY

SEQ ID NO: 190 - protein, 107 aa, synthetic construct
  REGION 1..107: note = Recombinant protein
EIVLTQSPATLSLSPGERATLSCRASQGISSYLHWYQQKPGEAPRLLIKYTSQPATGIPARFSGSGSGTDFTLTISSLEPEDVGVYYCQNGHSFPLTFGAGTKLELK

SEQ ID NO: 191 - protein, 11 aa, synthetic construct
  REGION 1..11: note = Recombinant protein
RASQGISSYLH

SEQ ID NO: 192 - protein, 7 aa, synthetic construct
  REGION 1..7: note = Recombinant protein
YTSQPAT

SEQ ID NO: 193 - protein, 9 aa, synthetic construct
  REGION 1..9: note = Recombinant protein
QNGHSFPLT

SEQ ID NO: 194 - protein, 122 aa, synthetic construct; identical to SEQ ID NO: 186
  REGION 1..122: note = Recombinant protein

SEQ ID NO: 195 - protein, 6 aa, synthetic construct
  REGION 1..6: note = Recombinant protein
SDSAWS

SEQ ID NO: 196 - protein, 16 aa, synthetic construct
  REGION 1..16: note = Recombinant protein
YIQYSGSTNYNPSLKS

SEQ ID NO: 197 - protein, 13 aa, synthetic construct
  REGION 1..13: note = Recombinant protein
SVNYYGNSFAVGY

SEQ ID NO: 198 - protein, 107 aa, synthetic construct
  REGION 1..107: note = Recombinant protein
EIVLTQSPATLSLSPGERATLSCRASQGISDYLHWYQQKPGQAPRLLIKYTSQPATGIPARFSGSGSGTDFTLTISSLEPEDVGVYYCQNGHSFPLTFGAGTKLELK

SEQ ID NO: 199 - protein, 11 aa, synthetic construct
  REGION 1..11: note = Recombinant protein
RASQGISDYLH

SEQ ID NO: 200 - protein, 7 aa, synthetic construct
  REGION 1..7: note = Recombinant protein
YTSQPAT

SEQ ID NO: 201 - protein, 9 aa, synthetic construct
  REGION 1..9: note = Recombinant protein
QNGHSFPLT

SEQ ID NO: 202 - protein, 122 aa, synthetic construct; identical to SEQ ID NO: 186
  REGION 1..122: note = Recombinant protein

SEQ ID NO: 203 - protein, 6 aa, synthetic construct
  REGION 1..6: note = Recombinant protein
SDSAWS

SEQ ID NO: 204 - protein, 16 aa, synthetic construct
  REGION 1..16: note = Recombinant protein
YIQYSGSTNYNPSLKS

SEQ ID NO: 205 - protein, 13 aa, synthetic construct
  REGION 1..13: note = Recombinant protein
SVNYYGNSFAVGY

SEQ ID NO: 206 - protein, 107 aa, synthetic construct
  REGION 1..107: note = Recombinant protein
EIVLTQSPATLSLSPGERATLSCRASQGISDYLHWYQQKPGEAPRLLIKYTSQPATGIPARFSGSGSGTDFTLTISSLEPEDVAVYYCQNGHSFPLTFGAGTKLELK

SEQ ID NO: 207 - protein, 11 aa, synthetic construct
  REGION 1..11: note = Recombinant protein
RASQGISDYLH

SEQ ID NO: 208 - protein, 7 aa, synthetic construct
  REGION 1..7: note = Recombinant protein
YTSQPAT

SEQ ID NO: 209 - protein, 9 aa, synthetic construct
  REGION 1..9: note = Recombinant protein
QNGHSFPLT

SEQ ID NO: 210 - protein, 122 aa, synthetic construct; identical to SEQ ID NO: 186
  REGION 1..122: note = Recombinant protein

SEQ ID NO: 211 - protein, 6 aa, synthetic construct
  REGION 1..6: note = Recombinant protein
SDSAWS

SEQ ID NO: 212 - protein, 16 aa, synthetic construct
  REGION 1..16: note = Recombinant protein
YIQYSGSTNYNPSLKS

SEQ ID NO: 213 - protein, 13 aa, synthetic construct
  REGION 1..13: note = Recombinant protein
SVNYYGNSFAVGY

SEQ ID NO: 214 - protein, 107 aa, synthetic construct
  REGION 1..107: note = Recombinant protein
EIVLTQSPATLSLSPGERATLSCRASQGVSDYLHWYQQKPGEAPRLLIKYTSQPATGIPARFSGSGSGTDFTLTISSLEPEDVGVYYCQNGHSFPLTFGAGTKLELK

SEQ ID NO: 215 - protein, 11 aa, synthetic construct
  REGION 1..11: note = Recombinant protein
RASQGVSDYLH

SEQ ID NO: 216 - protein, 7 aa, synthetic construct
  REGION 1..7: note = Recombinant protein
YTSQPAT

SEQ ID NO: 217 - protein, 9 aa, synthetic construct
  REGION 1..9: note = Recombinant protein
QNGHSFPLT

SEQ ID NO: 218 - protein, 122 aa, synthetic construct; identical to SEQ ID NO: 186
  REGION 1..122: note = Recombinant protein

SEQ ID NO: 219 - protein, 6 aa, synthetic construct
  REGION 1..6: note = Recombinant protein
SDSAWS

SEQ ID NO: 220 - protein, 16 aa, synthetic construct
  REGION 1..16: note = Recombinant protein
YIQYSGSTNYNPSLKS

SEQ ID NO: 221 - protein, 13 aa, synthetic construct
  REGION 1..13: note = Recombinant protein
SVNYYGNSFAVGY

SEQ ID NO: 222 - protein, 107 aa, synthetic construct
  REGION 1..107: note = Recombinant protein
EIVLTQSPATLSLSPGERATLSCRASQGISDYLHWYQQKPGEAPRLLIKYTSQPATGIPARFSGSGSGTDFTLTISSLEPEDVGVYYCQNGHSFPYTFGAGTKLELK

SEQ ID NO: 223 - protein, 11 aa, synthetic construct
  REGION 1..11: note = Recombinant protein
RASQGISDYLH

SEQ ID NO: 224 - protein, 7 aa, synthetic construct
  REGION 1..7: note = Recombinant protein
YTSQPAT

SEQ ID NO: 225 - protein, 9 aa, synthetic construct
  REGION 1..9: note = Recombinant protein
QNGHSFPYT

SEQ ID NO: 226 - protein, 122 aa, synthetic construct; identical to SEQ ID NO: 186
  REGION 1..122: note = Recombinant protein

SEQ ID NO: 227 - protein, 6 aa, synthetic construct
  REGION 1..6: note = Recombinant protein
SDSAWS

SEQ ID NO: 228 - protein, 16 aa, synthetic construct
  REGION 1..16: note = Recombinant protein
YIQYSGSTNYNPSLKS

SEQ ID NO: 229 - protein, 13 aa, synthetic construct
  REGION 1..13: note = Recombinant protein
SVNYYGNSFAVGY

SEQ ID NO: 230 - protein, 107 aa, synthetic construct
  REGION 1..107: note = Recombinant protein
EIVLTQSPATLSLSAGERATLSCRASQGISDYLHWYQQKSGEAPRLLIKYTSQPATGIPARFSGSGSGTDFTLTISSLEPEDVGVYYCQNGHSFPLTFGAGTKLELK

SEQ ID NO: 231 - protein, 11 aa, synthetic construct
  REGION 1..11: note = Recombinant protein
RASQGISDYLH

SEQ ID NO: 232 - protein, 7 aa, synthetic construct
  REGION 1..7: note = Recombinant protein
YTSQPAT

SEQ ID NO: 233 - protein, 9 aa, synthetic construct
  REGION 1..9: note = Recombinant protein
QNGHSFPLT

SEQ ID NO: 234 - protein, 122 aa, synthetic construct; identical to SEQ ID NO: 186
  REGION 1..122: note = Recombinant protein

SEQ ID NO: 235 - protein, 6 aa, synthetic construct
  REGION 1..6: note = Recombinant protein
SDSAWS

SEQ ID NO: 236 - protein, 16 aa, synthetic construct
  REGION 1..16: note = Recombinant protein
YIQYSGSTNYNPSLKS

SEQ ID NO: 237 - protein, 13 aa, synthetic construct
  REGION 1..13: note = Recombinant protein
SVNYYGNSFAVGY

SEQ ID NO: 238 - protein, 107 aa, synthetic construct
  REGION 1..107: note = Recombinant protein
EIVLTQSPATLSLSPGERATLSCRASQGISDYLHWYQQKPGEAPRLLIKYASQPATGIPARFSGSGSGTDFTLTISSLEPEDVGVYYCQNGHSFPLTFGAGTKLELK

SEQ ID NO: 239 - protein, 11 aa, synthetic construct
  REGION 1..11: note = Recombinant protein
RASQGISDYLH

SEQ ID NO: 240 - protein, 7 aa, synthetic construct
  REGION 1..7: note = Recombinant protein
YASQPAT

SEQ ID NO: 241 - protein, 9 aa, synthetic construct
  REGION 1..9: note = Recombinant protein
QNGHSFPLT

SEQ ID NO: 242 - protein, 122 aa, synthetic construct; identical to SEQ ID NO: 186
  REGION 1..122: note = Recombinant protein

SEQ ID NO: 243 - protein, 6 aa, synthetic construct
  REGION 1..6: note = Recombinant protein
SDSAWS

SEQ ID NO: 244 - protein, 16 aa, synthetic construct
  REGION 1..16: note = Recombinant protein
YIQYSGSTNYNPSLKS

SEQ ID NO: 245 - protein, 13 aa, synthetic construct
  REGION 1..13: note = Recombinant protein
SVNYYGNSFAVGY

SEQ ID NO: 246 - protein, 107 aa, synthetic construct
  REGION 1..107: note = Recombinant protein
EIVLTQSPATLSLSPGERATLSCRASQGISDYLHWYQQKPGEAPRLLIKYTSQPATGIPARFSGSGSGTDFTLTISSLEPEDFGVYYCQNGHSFPLTFGAGTKLELK

SEQ ID NO: 247 - protein, 11 aa, synthetic construct
  REGION 1..11: note = Recombinant protein
RASQGISDYLH

SEQ ID NO: 248 - protein, 7 aa, synthetic construct
  REGION 1..7: note = Recombinant protein
YTSQPAT

SEQ ID NO: 249 - protein, 9 aa, synthetic construct
  REGION 1..9: note = Recombinant protein
QNGHSFPLT

SEQ ID NO: 250 - protein, 122 aa, synthetic construct; identical to SEQ ID NO: 186
  REGION 1..122: note = Recombinant protein

SEQ ID NO: 251 - protein, 6 aa, synthetic construct
  REGION 1..6: note = Recombinant protein
SDSAWS

SEQ ID NO: 252 - protein, 16 aa, synthetic construct
  REGION 1..16: note = Recombinant protein
YIQYSGSTNYNPSLKS

SEQ ID NO: 253 - protein, 13 aa, synthetic construct
  REGION 1..13: note = Recombinant protein
SVNYYGNSFAVGY

SEQ ID NO: 254 - protein, 107 aa, synthetic construct; identical to SEQ ID NO: 102
  REGION 1..107: note = Recombinant protein

SEQ ID NO: 255 - protein, 11 aa, synthetic construct
  REGION 1..11: note = Recombinant protein
RASQGISDYLH

SEQ ID NO: 256 - protein, 7 aa, synthetic construct
  REGION 1..7: note = Recombinant protein
YTSQPAT

SEQ ID NO: 257 - protein, 9 aa, synthetic construct
  REGION 1..9: note = Recombinant protein
QNGHSFPLT

SEQ ID NO: 258 - protein, 122 aa, synthetic construct; identical to SEQ ID NO: 122
  REGION 1..122: note = Recombinant protein

SEQ ID NO: 259 - protein, 6 aa, synthetic construct
  REGION 1..6: note = Recombinant protein
SDSAWS

SEQ ID NO: 260 - protein, 16 aa, synthetic construct
  REGION 1..16: note = Recombinant protein
YIQYSGSTNYNPSLKS

SEQ ID NO: 261 - protein, 13 aa, synthetic construct
  REGION 1..13: note = Recombinant protein
SVNYYGNSFAVGY

SEQ ID NO: 262 - protein, 107 aa, synthetic construct; identical to SEQ ID NO: 102
  REGION 1..107: note = Recombinant protein

SEQ ID NO: 263 - protein, 11 aa, synthetic construct
  REGION 1..11: note = Recombinant protein
RASQGISDYLH

SEQ ID NO: 264 - protein, 7 aa, synthetic construct
  REGION 1..7: note = Recombinant protein
YTSQPAT

SEQ ID NO: 265 - protein, 9 aa, synthetic construct
  REGION 1..9: note = Recombinant protein
QNGHSFPLT

SEQ ID NO: 266 - protein, 122 aa, synthetic construct; identical to SEQ ID NO: 130
  REGION 1..122: note = Recombinant protein

SEQ ID NO: 267 - protein, 6 aa, synthetic construct
  REGION 1..6: note = Recombinant protein
SDSAWS

SEQ ID NO: 268 - protein, 16 aa, synthetic construct
  REGION 1..16: note = Recombinant protein
YIQYSGSTNYNPSLKS

SEQ ID NO: 269 - protein, 13 aa, synthetic construct
  REGION 1..13: note = Recombinant protein
SVNYYGNSFAVGY

SEQ ID NO: 270 - protein, 107 aa, synthetic construct; identical to SEQ ID NO: 102
  REGION 1..107: note = Recombinant protein

SEQ ID NO: 271 - protein, 11 aa, synthetic construct
  REGION 1..11: note = Recombinant protein
RASQGISDYLH

SEQ ID NO: 272 - protein, 7 aa, synthetic construct
  REGION 1..7: note = Recombinant protein
YTSQPAT

SEQ ID NO: 273 - protein, 9 aa, synthetic construct
  REGION 1..9: note = Recombinant protein
QNGHSFPLT

SEQ ID NO: 274 - protein, 122 aa, synthetic construct; identical to SEQ ID NO: 138
  REGION 1..122: note = Recombinant protein

SEQ ID NO: 275 - protein, 6 aa, synthetic construct
  REGION 1..6: note = Recombinant protein
SDSAWS

SEQ ID NO: 276 - protein, 16 aa, synthetic construct
  REGION 1..16: note = Recombinant protein
YIQYSGSTNYNPSLKS

SEQ ID NO: 277 - protein, 13 aa, synthetic construct
  REGION 1..13: note = Recombinant protein
SVNYYGNSFAVDY

SEQ ID NO: 278 - protein, 107 aa, synthetic construct; identical to SEQ ID NO: 102
  REGION 1..107: note = Recombinant protein

SEQ ID NO: 279 - protein, 11 aa, synthetic construct
  REGION 1..11: note = Recombinant protein
RASQGISDYLH

SEQ ID NO: 280 - protein, 7 aa, synthetic construct
  REGION 1..7: note = Recombinant protein
YTSQPAT

SEQ ID NO: 281 - protein, 9 aa, synthetic construct
  REGION 1..9: note = Recombinant protein
QNGHSFPLT

SEQ ID NO: 282 - protein, 122 aa, synthetic construct; identical to SEQ ID NO: 154
  REGION 1..122: note = Recombinant protein

SEQ ID NO: 283 - protein, 6 aa, synthetic construct
  REGION 1..6: note = Recombinant protein
SDSAWS

SEQ ID NO: 284 - protein, 16 aa, synthetic construct
  REGION 1..16: note = Recombinant protein
YIQYSGSTNYNPSLKS

SEQ ID NO: 285 - protein, 13 aa, synthetic construct
  REGION 1..13: note = Recombinant protein
SVNYYGNSFAVGY

SEQ ID NO: 286 - protein, 107 aa, synthetic construct; identical to SEQ ID NO: 102
  REGION 1..107: note = Recombinant protein

SEQ ID NO: 287 - protein, 11 aa, synthetic construct
  REGION 1..11: note = Recombinant protein
RASQGISDYLH

SEQ ID NO: 288 - protein, 7 aa, synthetic construct
  REGION 1..7: note = Recombinant protein
YTSQPAT

SEQ ID NO: 289 - protein, 9 aa, synthetic construct
  REGION 1..9: note = Recombinant protein
QNGHSFPLT

SEQ ID NO: 290 - protein, 122 aa, synthetic construct; identical to SEQ ID NO: 162
  REGION 1..122: note = Recombinant protein

SEQ ID NO: 291 - protein, 6 aa, synthetic construct
  REGION 1..6: note = Recombinant protein
SDSAWS

SEQ ID NO: 292 - protein, 16 aa, synthetic construct
  REGION 1..16: note = Recombinant protein
YIQYSGSTNYNPSLKS

SEQ ID NO: 293 - protein, 13 aa, synthetic construct
  REGION 1..13: note = Recombinant protein
SVNYYGNSFAVGY

SEQ ID NO: 294 - protein, 107 aa, synthetic construct; identical to SEQ ID NO: 190
  REGION 1..107: note = Recombinant protein

SEQ ID NO: 295 - protein, 11 aa, synthetic construct
  REGION 1..11: note = Recombinant protein
RASQGISSYLH

SEQ ID NO: 296 - protein, 7 aa, synthetic construct
  REGION 1..7: note = Recombinant protein
YTSQPAT

SEQ ID NO: 297 - protein, 9 aa, synthetic construct
  REGION 1..9: note = Recombinant protein
QNGHSFPLT

SEQ ID NO: 298 - protein, 122 aa, synthetic construct; identical to SEQ ID NO: 186
  REGION 1..122: note = Recombinant protein

SEQ ID NO: 299 - protein, 6 aa, synthetic construct
  REGION 1..6: note = Recombinant protein
SDSAWS

SEQ ID NO: 300 - protein, 16 aa, synthetic construct
  REGION 1..16: note = Recombinant protein
YIQYSGSTNYNPSLKS

SEQ ID NO: 301 - protein, 13 aa, synthetic construct
  REGION 1..13: note = Recombinant protein
SVNYYGNSFAVGY

SEQ ID NO: 302 - protein, 107 aa, synthetic construct; identical to SEQ ID NO: 246
  REGION 1..107: note = Recombinant protein

SEQ ID NO: 303 - protein, 11 aa, synthetic construct
  REGION 1..11: note = Recombinant protein
RASQGISDYLH

SEQ ID NO: 304 - protein, 7 aa, synthetic construct
  REGION 1..7: note = Recombinant protein
YTSQPAT

SEQ ID NO: 305 - protein, 9 aa, synthetic construct
  REGION 1..9: note = Recombinant protein
QNGHSFPLT

SEQ ID NO: 306 - protein, 122 aa, synthetic construct; identical to SEQ ID NO: 186
  REGION 1..122: note = Recombinant protein

SEQ ID NO: 307 - protein, 6 aa, synthetic construct
  REGION 1..6: note = Recombinant protein
SDSAWS

SEQ ID NO: 308 - protein, 16 aa, synthetic construct
  REGION 1..16: note = Recombinant protein
YIQYSGSTNYNPSLKS

SEQ ID NO: 309 - protein, 13 aa, synthetic construct
  REGION 1..13: note = Recombinant protein
SVNYYGNSFAVGY

SEQ ID NO: 310 - protein, 107 aa, synthetic construct
  REGION 1..107: note = Recombinant protein
EIVLTQSPATLSLSPGERATLSCRASQGISDYLAWYQQKPGQAPRLLIKYASQPATGIPARFSGSGSGTDFTLTISSLEPEDFAVYYCQNGHSFPLTFGQGTKLEIK

SEQ ID NO: 311 - protein, 11 aa, synthetic construct
  REGION 1..11: note = Recombinant protein
RASQGISDYLA

SEQ ID NO: 312 - protein, 7 aa, synthetic construct
  REGION 1..7: note = Recombinant protein
YASQPAT

SEQ ID NO: 313 - protein, 9 aa, synthetic construct
  REGION 1..9: note = Recombinant protein
QNGHSFPLT

SEQ ID NO: 314 - protein, 122 aa, synthetic construct
  REGION 1..122: note = Recombinant protein
QVQLQESGPGLVEPSDTLSLTCAVSGYSISSDSAWSWIRQPPGKGLEWIGYIQYSGSTNYNPSLKSRVTMSVDTSKNQFSLKLSSVTALDTAVYYCARSVNYYGNSFAVGPWGQGTLVTVSS

SEQ ID NO: 315 - protein, 6 aa, synthetic construct
  REGION 1..6: note = Recombinant protein
SDSAWS

SEQ ID NO: 316 - protein, 16 aa, synthetic construct
  REGION 1..16: note = Recombinant protein
YIQYSGSTNYNPSLKS

SEQ ID NO: 317 - protein, 13 aa, synthetic construct
  REGION 1..13: note = Recombinant protein
SVNYYGNSFAVGP

SEQ ID NO: 318 - protein, 107 aa, synthetic construct
  REGION 1..107: note = Recombinant protein
EIVLTQSPATLSLSPGERATLSCRASQGISDYLHWYQQKPGEAPRLLIKYTSQPATGIPARFSGSGSGTDFTLTISSLEPEDFAVYYCQNGHSFPLTFGQGTKLEIK

SEQ ID NO: 319 - protein, 11 aa, synthetic construct
  REGION 1..11: note = Recombinant protein
RASQGISDYLH

SEQ ID NO: 320 - protein, 7 aa, synthetic construct
  REGION 1..7: note = Recombinant protein
YTSQPAT

SEQ ID NO: 321 - protein, 9 aa, synthetic construct
  REGION 1..9: note = Recombinant protein
QNGHSFPLT

SEQ ID NO: 322 - protein, 122 aa, synthetic construct; identical to SEQ ID NO: 314
  REGION 1..122: note = Recombinant protein

SEQ ID NO: 323 - protein, 6 aa, synthetic construct
  REGION 1..6: note = Recombinant protein
SDSAWS

SEQ ID NO: 324 - protein, 16 aa, synthetic construct
  REGION 1..16: note = Recombinant protein
YIQYSGSTNYNPSLKS

SEQ ID NO: 325 - protein, 13 aa, synthetic construct
  REGION 1..13: note = Recombinant protein
SVNYYGNSFAVGP

SEQ ID NO: 326 - protein, 107 aa, synthetic construct; identical to SEQ ID NO: 246
  REGION 1..107: note = Recombinant protein

SEQ ID NO: 327 - protein, 11 aa, synthetic construct
  REGION 1..11: note = Recombinant protein
RASQGISDYLH

SEQ ID NO: 328 - protein, 7 aa, synthetic construct
  REGION 1..7: note = Recombinant protein
YTSQPAT

SEQ ID NO: 329 - protein, 9 aa, synthetic construct
  REGION 1..9: note = Recombinant protein
QNGHSFPLT

SEQ ID NO: 330 - protein, 122 aa, synthetic construct; identical to SEQ ID NO: 314
  REGION 1..122: note = Recombinant protein

SEQ ID NO: 331 - protein, 6 aa, synthetic construct
  REGION 1..6: note = Recombinant protein
SDSAWS

SEQ ID NO: 332 - protein, 16 aa, synthetic construct
  REGION 1..16: note = Recombinant protein
YIQYSGSTNYNPSLKS

SEQ ID NO: 333 - protein, 13 aa, synthetic construct
  REGION 1..13: note = Recombinant protein
SVNYYGNSFAVGP

SEQ ID NO: 334 - protein, 107 aa, synthetic construct
  REGION 1..107: note = Recombinant protein
EIVLTQSPATLSLSPGERATLSCRASQGISDYLHWYQQKPGQAPRLLIKYASQPATGIPARFSGSGSGTDFTLTISSLEPEDFAVYYCQNGHSFPLTFGQGTKLEIK

SEQ ID NO: 335 - protein, 11 aa, synthetic construct
  REGION 1..11: note = Recombinant protein
RASQGISDYLH

SEQ ID NO: 336 - protein, 7 aa, synthetic construct
  REGION 1..7: note = Recombinant protein
YASQPAT

SEQ ID NO: 337 - protein, 9 aa, synthetic construct
  REGION 1..9: note = Recombinant protein
QNGHSFPLT

SEQ ID NO: 338 - protein, 122 aa, synthetic construct; identical to SEQ ID NO: 314
  REGION 1..122: note = Recombinant protein

SEQ ID NO: 339 - protein, 6 aa, synthetic construct
  REGION 1..6: note = Recombinant protein
SDSAWS

SEQ ID NO: 340 - protein, 16 aa, synthetic construct
  REGION 1..16: note = Recombinant protein
YIQYSGSTNYNPSLKS

SEQ ID NO: 341 - protein, 13 aa, synthetic construct
  REGION 1..13: note = Recombinant protein
SVNYYGNSFAVGP

SEQ ID NO: 342 - protein, 107 aa, synthetic construct
  REGION 1..107: note = Recombinant protein
EIVLTQSPATLSLSPGERATLSCRASQGISDYLHWYQQKPGQAPRLLIKYTSQPATGIPARFSGSGSGRDFTLTISSLEPEDFAVYYCQNGHSFPLTFGAGTKLELK

SEQ ID NO: 343 - protein, 11 aa, synthetic construct
  REGION 1..11: note = Recombinant protein
RASQGISDYLH

SEQ ID NO: 344 - protein, 7 aa, synthetic construct
  REGION 1..7: note = Recombinant protein
YTSQPAT

SEQ ID NO: 345 - protein, 9 aa, synthetic construct
  REGION 1..9: note = Recombinant protein
QNGHSFPLT

SEQ ID NO: 346 - protein, 122 aa, synthetic construct; identical to SEQ ID NO: 314
  REGION 1..122: note = Recombinant protein

SEQ ID NO: 347 - protein, 6 aa, synthetic construct
  REGION 1..6: note = Recombinant protein
SDSAWS

SEQ ID NO: 348 - protein, 16 aa, synthetic construct
  REGION 1..16: note = Recombinant protein
YIQYSGSTNYNPSLKS

SEQ ID NO: 349 - protein, 13 aa, synthetic construct
  REGION 1..13: note = Recombinant protein
SVNYYGNSFAVGP

SEQ ID NO: 350 - protein, 107 aa, synthetic construct
  REGION 1..107: note = Recombinant protein
EIVLTQSPATLSLSPGERATLSCRASQGISDYLHWYQQKPGQAPRLLIKYTSQPATGIPARFSGSGSGTDFTLTISSLEPEDFAVYYCQNGHSFPLTFGAGTKLELK

SEQ ID NO: 351 - protein, 11 aa, synthetic construct
  REGION 1..11: note = Recombinant protein
RASQGISDYLH

SEQ ID NO: 352 - protein, 7 aa, synthetic construct
  REGION 1..7: note = Recombinant protein
YTSQPAT

SEQ ID NO: 353 - protein, 9 aa, synthetic construct
  REGION 1..9: note = Recombinant protein
QNGHSFPLT

SEQ ID NO: 354 - protein, 122 aa, synthetic construct; identical to SEQ ID NO: 314
  REGION 1..122: note = Recombinant protein

SEQ ID NO: 355 - protein, 6 aa, synthetic construct
  REGION 1..6: note = Recombinant protein
SDSAWS

SEQ ID NO: 356 - protein, 16 aa, synthetic construct
  REGION 1..16: note = Recombinant protein
YIQYSGSTNYNPSLKS

SEQ ID NO: 357 - protein, 13 aa, synthetic construct
  REGION 1..13: note = Recombinant protein
SVNYYGNSFAVGP

SEQ ID NO: 358 - protein, 107 aa, synthetic construct
  REGION 1..107: note = Recombinant protein
EIVLTQSPATLSLSPGERATLSCRASQGISDYLHWYQQKPGQAPRLLIKYTSQPATGIPARFSGSGSGTDFTLTISSLEPEDFAVYYCQNGHSFPLTFGQGTKLEIK

SEQ ID NO: 359 - protein, 11 aa, synthetic construct
  REGION 1..11: note = Recombinant protein
RASQGISDYLH

SEQ ID NO: 360 - protein, 7 aa, synthetic construct
  REGION 1..7: note = Recombinant protein
YTSQPAT

SEQ ID NO: 361 - protein, 9 aa, synthetic construct
  REGION 1..9: note = Recombinant protein
QNGHSFPLT

SEQ ID NO: 362 - protein, 122 aa, synthetic construct; identical to SEQ ID NO: 314
  REGION 1..122: note = Recombinant protein

SEQ ID NO: 363 - protein, 6 aa, synthetic construct
  REGION 1..6: note = Recombinant protein
SDSAWS

SEQ ID NO: 364 - protein, 16 aa, synthetic construct
  REGION 1..16: note = Recombinant protein
YIQYSGSTNYNPSLKS

SEQ ID NO: 365 - protein, 13 aa, synthetic construct
  REGION 1..13: note = Recombinant protein
SVNYYGNSFAVGP

SEQ ID NO: 366 - protein, 107 aa, synthetic construct
  REGION 1..107: note = Recombinant protein
EIVLTQSPATLSLSPGERATLSCRASQGLSDYLAWYQQKPGEAPRLLIKYASQPATGIPARFSGSGSGTDFTLTISSLEPEDFAVYYCQNGHSFPLTFGQGTKLEIK

SEQ ID NO: 367 - protein, 11 aa, synthetic construct
  REGION 1..11: note = Recombinant protein
RASQGLSDYLA

SEQ ID NO: 368 - protein, 7 aa, synthetic construct
  REGION 1..7: note = Recombinant protein
YASQPAT

SEQ ID NO: 369 - protein, 9 aa, synthetic construct
  REGION 1..9: note = Recombinant protein
QNGHSFPLT

SEQ ID NO: 370 - protein, 122 aa, synthetic construct; identical to SEQ ID NO: 314
  REGION 1..122: note = Recombinant protein

SEQ ID NO: 371 - protein, 6 aa, synthetic construct
  REGION 1..6: note = Recombinant protein
SDSAWS

SEQ ID NO: 372 - protein, 16 aa, synthetic construct
  REGION 1..16: note = Recombinant protein
YIQYSGSTNYNPSLKS

SEQ ID NO: 373 - protein, 13 aa, synthetic construct
  REGION 1..13: note = Recombinant protein
SVNYYGNSFAVGP

SEQ ID NO: 374 - protein, 107 aa, synthetic construct
  REGION 1..107: note = Recombinant protein
EIVLTQSPATLSLSPGERATLSCRASQGLSDYLAWYQQKPGQAPRLLIKYTSQPATGIPARFSGSGSGTDFTLTISSLEPEDFAVYYCQNGHSFPLTFGQGTKLEIK

SEQ ID NO: 375 - protein, 11 aa, synthetic construct
  REGION 1..11: note = Recombinant protein
RASQGLSDYLA

SEQ ID NO: 376 - protein, 7 aa, synthetic construct
  REGION 1..7: note = Recombinant protein
YTSQPAT

SEQ ID NO: 377 - protein, 9 aa, synthetic construct
  REGION 1..9: note = Recombinant protein
QNGHSFPLT

SEQ ID NO: 378 - protein, 122 aa, synthetic construct; identical to SEQ ID NO: 314
  REGION 1..122: note = Recombinant protein

SEQ ID NO: 379 - protein, 6 aa, synthetic construct
  REGION 1..6: note = Recombinant protein
SDSAWS

SEQ ID NO: 380 - protein, 16 aa, synthetic construct
  REGION 1..16: note = Recombinant protein
YIQYSGSTNYNPSLKS

SEQ ID NO: 381 - protein, 13 aa, synthetic construct
  REGION 1..13: note = Recombinant protein
SVNYYGNSFAVGP

SEQ ID NO: 382 - protein, 107 aa, synthetic construct
  REGION 1..107: note = Recombinant protein
EIVLTQSPATLSLSPGERATLSCRASQGLSDYLHWYQQKPGEAPRLLIKYASQPATGIPARFSGSGSGTDFTLTISSLEPEDFAVYYCQNGHSFPLTFGQGTKLEIK

SEQ ID NO: 383 - protein, 11 aa, synthetic construct
  REGION 1..11: note = Recombinant protein
RASQGLSDYLH

SEQ ID NO: 384 - protein, 7 aa, synthetic construct
  REGION 1..7: note = Recombinant protein
YASQPAT

SEQ ID NO: 385 - protein, 9 aa, synthetic construct
  REGION 1..9: note = Recombinant protein
QNGHSFPLT

SEQ ID NO: 386 - protein, 122 aa, synthetic construct; identical to SEQ ID NO: 314
  REGION 1..122: note = Recombinant protein

SEQ ID NO: 387 - protein, 6 aa, synthetic construct
  REGION 1..6: note = Recombinant protein
SDSAWS

SEQ ID NO: 388 - protein, 16 aa, synthetic construct
  REGION 1..16: note = Recombinant protein
YIQYSGSTNYNPSLKS

SEQ ID NO: 389 - protein, 13 aa, synthetic construct
  REGION 1..13: note = Recombinant protein
SVNYYGNSFAVGP

SEQ ID NO: 390 - protein, 107 aa, synthetic construct
  REGION 1..107: note = Recombinant protein
EIVLTQSPATLSLSPGERATLSCRASQGVSDYLAWYQQKPGEAPRLLIKYASQPATGIPARFSGSGSGTDFTLTISSLEPEDFAVYYCQNGHSFPLTFGQGTKLEIK

SEQ ID NO: 391 - protein, 11 aa, synthetic construct
  REGION 1..11: note = Recombinant protein
RASQGVSDYLA

SEQ ID NO: 392 - protein, 7 aa, synthetic construct
  REGION 1..7: note = Recombinant protein
YASQPAT

SEQ ID NO: 393 - protein, 9 aa, synthetic construct
  REGION 1..9: note = Recombinant protein
QNGHSFPLT

SEQ ID NO: 394 - protein, 122 aa, synthetic construct; identical to SEQ ID NO: 314
  REGION 1..122: note = Recombinant protein

SEQ ID NO: 395 - protein, 6 aa, synthetic construct
  REGION 1..6: note = Recombinant protein
SDSAWS

SEQ ID NO: 396 - protein, 16 aa, synthetic construct
  REGION 1..16: note = Recombinant protein
YIQYSGSTNYNPSLKS

SEQ ID NO: 397 - protein, 13 aa, synthetic construct
  REGION 1..13: note = Recombinant protein
SVNYYGNSFAVGP

SEQ ID NO: 398 - protein, 107 aa, synthetic construct
  REGION 1..107: note = Recombinant protein
EIVLTQSPATLSLSPGERATLSCRASQGVSDYLAWYQQKPGEAPRLLIKYTSQPATGIPARFSGSGSGTDFTLTISSLEPEDFAVYYCQNGHSFPLTFGQGTKLEIK

SEQ ID NO: 399 - protein, 11 aa, synthetic construct
  REGION 1..11: note = Recombinant protein
RASQGVSDYLA

SEQ ID NO: 400 - protein, 7 aa, synthetic construct
  REGION 1..7: note = Recombinant protein
YTSQPAT

SEQ ID NO: 401 - protein, 9 aa, synthetic construct
  REGION 1..9: note = Recombinant protein
QNGHSFPLT

SEQ ID NO: 402 - protein, 122 aa, synthetic construct; identical to SEQ ID NO: 314
  REGION 1..122: note = Recombinant protein

SEQ ID NO: 403 - protein, 6 aa, synthetic construct
  REGION 1..6: note = Recombinant protein
SDSAWS

SEQ ID NO: 404 - protein, 16 aa, synthetic construct
  REGION 1..16: note = Recombinant protein
YIQYSGSTNYNPSLKS

SEQ ID NO: 405 - protein, 13 aa, synthetic construct
  REGION 1..13: note = Recombinant protein
SVNYYGNSFAVGP

SEQ ID NO: 406 - protein, 107 aa, synthetic construct
  REGION 1..107: note = Recombinant protein
EIVLTQSPATLSLSPGERATLSCRASQGVSDYLAWYQQKPGQAPRLLIKYASQPATGIPARFSGSGSGTDFTLTISSLEPEDFAVYYCQNGHSFPLTFGAGTKLEIK

SEQ ID NO: 407 - protein, 11 aa, synthetic construct
  REGION 1..11: note = Recombinant protein
RASQGVSDYLA

SEQ ID NO: 408 - protein, 7 aa, synthetic construct
  REGION 1..7: note = Recombinant protein
YASQPAT

SEQ ID NO: 409 - protein, 9 aa, synthetic construct
  REGION 1..9: note = Recombinant protein
QNGHSFPLT

SEQ ID NO: 410 - protein, 122 aa, synthetic construct; identical to SEQ ID NO: 314
  REGION 1..122: note = Recombinant protein

SEQ ID NO: 411 - protein, 6 aa, synthetic construct
  REGION 1..6: note = Recombinant protein
SDSAWS

SEQ ID NO: 412 - protein, 16 aa, synthetic construct
  REGION 1..16: note = Recombinant protein
YIQYSGSTNYNPSLKS

SEQ ID NO: 413 - protein, 13 aa, synthetic construct
  REGION 1..13: note = Recombinant protein
SVNYYGNSFAVGP

SEQ ID NO: 414 - protein, 107 aa, synthetic construct
  REGION 1..107: note = Recombinant protein
EIVLTQSPATLSLSPGERATLSCRASQGVSDYLAWYQQKPGQAPRLLIKYASQPATGIPARFSGSGSGTDFTLTISSLEPEDFAVYYCQNGHSFPLTFGQGTKLEIK

SEQ ID NO: 415 - protein, 11 aa, synthetic construct
  REGION 1..11: note = Recombinant protein
RASQGVSDYLA

SEQ ID NO: 416 - protein, 7 aa, synthetic construct
  REGION 1..7: note = Recombinant protein
YASQPAT

SEQ ID NO: 417 - protein, 9 aa, synthetic construct
  REGION 1..9: note = Recombinant protein
QNGHSFPLT

SEQ ID NO: 418 - protein, 122 aa, synthetic construct; identical to SEQ ID NO: 314
  REGION 1..122: note = Recombinant protein

SEQ ID NO: 419 - protein, 6 aa, synthetic construct
  REGION 1..6: note = Recombinant protein
SDSAWS

SEQ ID NO: 420 - protein, 16 aa, synthetic construct
  REGION 1..16: note = Recombinant protein
YIQYSGSTNYNPSLKS

SEQ ID NO: 421 - protein, 13 aa, synthetic construct
  REGION 1..13: note = Recombinant protein
SVNYYGNSFAVGP

SEQ ID NO: 422 - protein, 107 aa, synthetic construct
  REGION 1..107: note = Recombinant protein
EIVLTQSPATLSLSPGERATLSCRASQGVSDYLAWYQQKPGQAPRLLIKYASQPATGIPARFSGSGSGTDFTLTISSLEPEDFAVYYCQQGHSFPLTFGQGTKLEIK

SEQ ID NO: 423 - protein, 11 aa, synthetic construct
  REGION 1..11: note = Recombinant protein
RASQGVSDYLA

SEQ ID NO: 424 - protein, 7 aa, synthetic construct
  REGION 1..7: note = Recombinant protein
YASQPAT

SEQ ID NO: 425 - protein, 9 aa, synthetic construct
  REGION 1..9: note = Recombinant protein
QQGHSFPLT

SEQ ID NO: 426 - protein, 122 aa, synthetic construct; identical to SEQ ID NO: 314
  REGION 1..122: note = Recombinant protein

SEQ ID NO: 427 - protein, 6 aa, synthetic construct
  REGION 1..6: note = Recombinant protein
SDSAWS

SEQ ID NO: 428 - protein, 16 aa, synthetic construct
  REGION 1..16: note = Recombinant protein
YIQYSGSTNYNPSLKS

SEQ ID NO: 429 - protein, 13 aa, synthetic construct
  REGION 1..13: note = Recombinant protein
SVNYYGNSFAVGP

SEQ ID NO: 430 - protein, 107 aa, synthetic construct
  REGION 1..107: note = Recombinant protein
EIVLTQSPATLSLSPGERATLSCRASQGVSDYLAWYQQKPGQAPRLLIKYASQPATGIPARFSGSGSGTDFTLTISSLEPEDFGVYYCQNGHSFPLTFGQGTKLEIK

SEQ ID NO: 431 - protein, 11 aa, synthetic construct
  REGION 1..11: note = Recombinant protein
RASQGVSDYLA

SEQ ID NO: 432 - protein, 7 aa, synthetic construct
  REGION 1..7: note = Recombinant protein
YASQPAT

SEQ ID NO: 433 - protein, 9 aa, synthetic construct
  REGION 1..9: note = Recombinant protein
QNGHSFPLT

SEQ ID NO: 434 - protein, 122 aa, synthetic construct; identical to SEQ ID NO: 314
  REGION 1..122: note = Recombinant protein

SEQ ID NO: 435 - protein, 6 aa, synthetic construct
  REGION 1..6: note = Recombinant protein
SDSAWS

SEQ ID NO: 436 - protein, 16 aa, synthetic construct
  REGION 1..16: note = Recombinant protein
YIQYSGSTNYNPSLKS

SEQ ID NO: 437 - protein, 13 aa, synthetic construct
  REGION 1..13: note = Recombinant protein
SVNYYGNSFAVGP

SEQ ID NO: 438 - protein, 107 aa, synthetic construct
  REGION 1..107: note = Recombinant protein
EIVLTQSPATLSLSPGERATLSCRASQGVSDYLAWYQQKPGQAPRLLIKYASQPATGIPARFSGSGSGTDFTLTISSLEPEDVAVYYCQNGHSFPLTFGQGTKLEIK

SEQ ID NO: 439 - protein, 11 aa, synthetic construct
  REGION 1..11: note = Recombinant protein
RASQGVSDYLA

SEQ ID NO: 440 - protein, 7 aa, synthetic construct
  REGION 1..7: note = Recombinant protein
YASQPAT

SEQ ID NO: 441 - protein, 9 aa, synthetic construct
  REGION 1..9: note = Recombinant protein
QNGHSFPLT

SEQ ID NO: 442 - protein, 122 aa, synthetic construct; identical to SEQ ID NO: 314
  REGION 1..122: note = Recombinant protein

SEQ ID NO: 443 - protein, 6 aa, synthetic construct
  REGION 1..6: note = Recombinant protein
SDSAWS

SEQ ID NO: 444 - protein, 16 aa, synthetic construct
  REGION 1..16: note = Recombinant protein
YIQYSGSTNYNPSLKS

SEQ ID NO: 445 - protein, 13 aa, synthetic construct
  REGION 1..13: note = Recombinant protein
SVNYYGNSFAVGP

SEQ ID NO: 446 - protein, 107 aa, synthetic construct
  REGION 1..107: note = Recombinant protein
EIVLTQSPATLSLSPGERATLSCRASQGVSDYLAWYQQKPGQAPRLLIKYTSQPATGIPARFSGSGSGTDFTLTISSLEPEDFAVYYCQNGHSFPLTFGQGTKLEIK

SEQ ID NO: 447 - protein, 11 aa, synthetic construct
  REGION 1..11: note = Recombinant protein
RASQGVSDYLA

SEQ ID NO: 448 - protein, 7 aa, synthetic construct
  REGION 1..7: note = Recombinant protein
YTSQPAT

SEQ ID NO: 449 - protein, 9 aa, synthetic construct
  REGION 1..9: note = Recombinant protein
QNGHSFPLT

SEQ ID NO: 450 - protein, 122 aa, synthetic construct; identical to SEQ ID NO: 314
  REGION 1..122: note = Recombinant protein

SEQ ID NO: 451 - protein, 6 aa, synthetic construct
  REGION 1..6: note = Recombinant protein
SDSAWS

SEQ ID NO: 452 - protein, 16 aa, synthetic construct
  REGION 1..16: note = Recombinant protein
YIQYSGSTNYNPSLKS

SEQ ID NO: 453 - protein, 13 aa, synthetic construct
  REGION 1..13: note = Recombinant protein
SVNYYGNSFAVGP

SEQ ID NO: 454 - protein, 107 aa, synthetic construct
  REGION 1..107: note = Recombinant protein
EIVLTQSPATLSLSPGERATLSCRASQGVSDYLHWYQQKPGEAPRLLIKYASQPATGIPARFSGSGSGTDFTLTISSLEPEDFAVYYCQNGHSFPLTFGQGTKLEIK

SEQ ID NO: 455 - protein, 11 aa, synthetic construct
  REGION 1..11: note = Recombinant protein
RASQGVSDYLH

SEQ ID NO: 456 - protein, 7 aa, synthetic construct
  REGION 1..7: note = Recombinant protein
YASQPAT

SEQ ID NO: 457 - protein, 9 aa, synthetic construct
  REGION 1..9: note = Recombinant protein
QNGHSFPLT

SEQ ID NO: 458 - protein, 122 aa, synthetic construct; identical to SEQ ID NO: 314
  REGION 1..122: note = Recombinant protein

SEQ ID NO: 459 - protein, 6 aa, synthetic construct
  REGION 1..6: note = Recombinant protein
SDSAWS

SEQ ID NO: 460 - protein, 16 aa, synthetic construct
  REGION 1..16: note = Recombinant protein
YIQYSGSTNYNPSLKS

SEQ ID NO: 461 - protein, 13 aa, synthetic construct
  REGION 1..13: note = Recombinant protein
SVNYYGNSFAVGP

SEQ ID NO: 462 - protein, 107 aa, synthetic construct
  REGION 1..107: note = Recombinant protein
EIVLTQSPATLSLSPGERATLSCRASQGVSDYLHWYQQKPGQAPRLLIKYASQPATGIPARFSGSGSGTDFTLTISSLEPEDFAVYYCQNGHSFPLTFGAGTKLEIK

SEQ ID NO: 463 - protein, 11 aa, synthetic construct
  REGION 1..11: note = Recombinant protein
RASQGVSDYLH

SEQ ID NO: 464 - protein, 7 aa, synthetic construct
  REGION 1..7: note = Recombinant protein
YASQPAT

SEQ ID NO: 465 - protein, 9 aa, synthetic construct
  REGION 1..9: note = Recombinant protein
QNGHSFPLT

SEQ ID NO: 466 - protein, 122 aa, synthetic construct; identical to SEQ ID NO: 314
  REGION 1..122: note = Recombinant protein

SEQ ID NO: 467 - protein, 6 aa, synthetic construct
  REGION 1..6: note = Recombinant protein
SDSAWS

SEQ ID NO: 468 - protein, 16 aa, synthetic construct
  REGION 1..16: note = Recombinant protein
YIQYSGSTNYNPSLKS

SEQ ID NO: 469 - protein, 13 aa, synthetic construct
  REGION 1..13: note = Recombinant protein
SVNYYGNSFAVGP

SEQ ID NO: 470 - protein, 107 aa, synthetic construct
  REGION 1..107: note = Recombinant protein
EIVLTQSPATLSLSPGERATLSCRASQGVSDYLHWYQQKPGQAPRLLIKYASQPATGIPARFSGSGSGTDFTLTISSLEPEDFAVYYCQNGHSFPLTFGQGTKLEIK

SEQ ID NO: 471 - protein, 11 aa, synthetic construct
  REGION 1..11: note = Recombinant protein
RASQGVSDYLH

SEQ ID NO: 472 - protein, 7 aa, synthetic construct
  REGION 1..7: note = Recombinant protein
YASQPAT

SEQ ID NO: 473 - protein, 9 aa, synthetic construct
  REGION 1..9: note = Recombinant protein
QNGHSFPLT

SEQ ID NO: 474 - protein, 122 aa, synthetic construct; identical to SEQ ID NO: 314
  REGION 1..122: note = Recombinant protein

SEQ ID NO: 475 - protein, 6 aa, synthetic construct
  REGION 1..6: note = Recombinant protein
SDSAWS

SEQ ID NO: 476 - protein, 16 aa, synthetic construct
  REGION 1..16: note = Recombinant protein
YIQYSGSTNYNPSLKS

SEQ ID NO: 477 - protein, 13 aa, synthetic construct
  REGION 1..13: note = Recombinant protein
SVNYYGNSFAVGP

SEQ ID NO: 478 - protein, 107 aa, synthetic construct
  REGION 1..107: note = Recombinant protein
EIVLTQSPATLSLSPGERATLSCRASQGVSDYLHWYQQKPGQAPRLLIKYASQPATGIPARFSGSGSGTDFTLTISSLEPEDFAVYYCQQGHSFPLTFGQGTKLEIK

SEQ ID NO: 479 - protein, 11 aa, synthetic construct
  REGION 1..11: note = Recombinant protein
RASQGVSDYLH

SEQ ID NO: 480 - protein, 7 aa, synthetic construct
  REGION 1..7: note = Recombinant protein
YASQPAT

SEQ ID NO: 481 - protein, 9 aa, synthetic construct
  REGION 1..9: note = Recombinant protein
QQGHSFPLT

SEQ ID NO: 482 - protein, 122 aa, synthetic construct; identical to SEQ ID NO: 314
  REGION 1..122: note = Recombinant protein

SEQ ID NO: 483 - protein, 6 aa, synthetic construct
  REGION 1..6: note = Recombinant protein
SDSAWS

SEQ ID NO: 484 - protein, 16 aa, synthetic construct
  REGION 1..16: note = Recombinant protein
YIQYSGSTNYNPSLKS

SEQ ID NO: 485 - protein, 13 aa, synthetic construct
  REGION 1..13: note = Recombinant protein
SVNYYGNSFAVGP

SEQ ID NO: 486 - protein, 107 aa, synthetic construct
  REGION 1..107: note = Recombinant protein
EIVLTQSPATLSLSPGERATLSCRASQGVSDYLHWYQQKPGQAPRLLIKYASQPATGIPARFSGSGSGTDFTLTISSLEPEDFGVYYCQNGHSFPLTFGQGTKLEIK

SEQ ID NO: 487 - protein, 11 aa, synthetic construct
  REGION 1..11: note = Recombinant protein
RASQGVSDYLH

SEQ ID NO: 488 - protein, 7 aa, synthetic construct
  REGION 1..7: note = Recombinant protein
YASQPAT

SEQ ID NO: 489 - protein, 9 aa, synthetic construct
  REGION 1..9: note = Recombinant protein
QNGHSFPLT

SEQ ID NO: 490 - protein, 122 aa, synthetic construct; identical to SEQ ID NO: 314
  REGION 1..122: note = Recombinant protein

SEQ ID NO: 491 - protein, 6 aa, synthetic construct
  REGION 1..6: note = Recombinant protein
SDSAWS

SEQ ID NO: 492 - protein, 16 aa, synthetic construct
  REGION 1..16: note = Recombinant protein
YIQYSGSTNYNPSLKS

SEQ ID NO: 493 - protein, 13 aa, synthetic construct
  REGION 1..13: note = Recombinant protein
SVNYYGNSFAVGP

SEQ ID NO: 494 - protein, 107 aa, synthetic construct
  REGION 1..107: note = Recombinant protein
EIVLTQSPATLSLSPGERATLSCRASQGVSDYLHWYQQKPGQAPRLLIKYASQPATGIPARFSGSGSGTDFTLTISSLEPEDVAVYYCQNGHSFPLTFGQGTKLEIK

SEQ ID NO: 495 - protein, 11 aa, synthetic construct
  REGION 1..11: note = Recombinant protein
RASQGVSDYLH

SEQ ID NO: 496 - protein, 7 aa, synthetic construct
  REGION 1..7: note = Recombinant protein
YASQPAT

SEQ ID NO: 497 - protein, 9 aa, synthetic construct
  REGION 1..9: note = Recombinant protein
QNGHSFPLT

SEQ ID NO: 498 - protein, 122 aa, synthetic construct; identical to SEQ ID NO: 314
  REGION 1..122: note = Recombinant protein

SEQ ID NO: 499 - protein, 6 aa, synthetic construct
  REGION 1..6: note = Recombinant protein
SDSAWS

SEQ ID NO: 500 - protein, 16 aa, synthetic construct
  REGION 1..16: note = Recombinant protein
YIQYSGSTNYNPSLKS

SEQ ID NO: 501 - protein, 13 aa, synthetic construct
  REGION 1..13: note = Recombinant protein
SVNYYGNSFAVGP

SEQ ID NO: 502 - protein, 107 aa, synthetic construct
  REGION 1..107: note = Recombinant protein
EIVLTQSPATLSLSPGERATLSCRASQGVSDYLHWYQQKPGQAPRLLIKYTSQPATGIPARFSGSGSGTDFTLTISSLEPEDFAVYYCQNGHSFPLTFGQGTKLEIK

SEQ ID NO: 503 - protein, 11 aa, synthetic construct
  REGION 1..11: note = Recombinant protein
RASQGVSDYLH

SEQ ID NO: 504 - protein, 7 aa, synthetic construct
  REGION 1..7: note = Recombinant protein
YTSQPAT

SEQ ID NO: 505 - protein, 9 aa, synthetic construct
  REGION 1..9: note = Recombinant protein
QNGHSFPLT

SEQ ID NO: 506 - protein, 122 aa, synthetic construct; identical to SEQ ID NO: 314
  REGION 1..122: note = Recombinant protein

SEQ ID NO: 507 - protein, 6 aa, synthetic construct
  REGION 1..6: note = Recombinant protein
SDSAWS

SEQ ID NO: 508 - protein, 16 aa, synthetic construct
  REGION 1..16: note = Recombinant protein
YIQYSGSTNYNPSLKS

SEQ ID NO: 509 - protein, 13 aa, synthetic construct
  REGION 1..13: note = Recombinant protein
SVNYYGNSFAVGP

SEQ ID NO: 510 - protein, 107 aa, synthetic construct; identical to SEQ ID NO: 310
  REGION 1..107: note = Recombinant protein

SEQ ID NO: 511 - protein, 11 aa, synthetic construct
  REGION 1..11: note = Recombinant protein
RASQGISDYLA

SEQ ID NO: 512 - protein, 7 aa, synthetic construct
  REGION 1..7: note = Recombinant protein
YASQPAT

SEQ ID NO: 513 - protein, 9 aa, synthetic construct
  REGION 1..9: note = Recombinant protein
QNGHSFPLT

SEQ ID NO: 514 - protein, 122 aa, synthetic construct
  REGION 1..122: note = Recombinant protein
QVQLQESGPGLVEPSDTLSLTCAVSGYSITSDSAWSWIRQPPGKKLEWIGYIQYSGSTNYNPSLKSRVTMSRDTSKNQFSLKLSSVTALDTAVYYCARSVNYYGNSFAVGYWGQGTLVTVSS

SEQ ID NO: 515 - protein, 6 aa, synthetic construct
  REGION 1..6: note = Recombinant protein
SDSAWS

SEQ ID NO: 516 - protein, 16 aa, synthetic construct
  REGION 1..16: note = Recombinant protein
YIQYSGSTNYNPSLKS

SEQ ID NO: 517 - protein, 13 aa, synthetic construct
  REGION 1..13: note = Recombinant protein
SVNYYGNSFAVGY

SEQ ID NO: 518 - protein, 107 aa, synthetic construct; identical to SEQ ID NO: 310
  REGION 1..107: note = Recombinant protein

SEQ ID NO: 519 - protein, 11 aa, synthetic construct
  REGION 1..11: note = Recombinant protein
RASQGISDYLA

SEQ ID NO: 520 - protein, 7 aa, synthetic construct
  REGION 1..7: note = Recombinant protein
YASQPAT

SEQ ID NO: 521 - protein, 9 aa, synthetic construct
  REGION 1..9: note = Recombinant protein
QNGHSFPLT

SEQ ID NO: 522 - protein, 122 aa, synthetic construct
  REGION 1..122: note = Recombinant protein
QVQLQESGPGLVEPSQTLSLTCTVSGYSISSDSAWSWIRQPPGKGLEWIGYIQYSGSTNYNPSLKSRVTISRDTSKNQFSLKLSSVTAADTAVYYCARSVNYYGNSFAVGPWGQGTLVTVSS

SEQ ID NO: 523 - protein, 6 aa, synthetic construct
  REGION 1..6: note = Recombinant protein
SDSAWS

SEQ ID NO: 524 - protein, 16 aa, synthetic construct
  REGION 1..16: note = Recombinant protein
YIQYSGSTNYNPSLKS

SEQ ID NO: 525 - protein, 13 aa, synthetic construct
  REGION 1..13: note = Recombinant protein
SVNYYGNSFAVGP

SEQ ID NO: 526 - protein, 107 aa, synthetic construct; identical to SEQ ID NO: 310
  REGION 1..107: note = Recombinant protein

SEQ ID NO: 527 - protein, 11 aa, synthetic construct
  REGION 1..11: note = Recombinant protein
RASQGISDYLA

SEQ ID NO: 528 - protein, 7 aa, synthetic construct
  REGION 1..7: note = Recombinant protein
YASQPAT

SEQ ID NO: 529 - protein, 9 aa, synthetic construct
  REGION 1..9: note = Recombinant protein
QNGHSFPLT

SEQ ID NO: 530 - protein, 122 aa, synthetic construct
  REGION 1..122: note = Recombinant protein
QVQLQESGPGLVEPSQTLSLTCTVSGYSISSDSAWSWIRQPPGKGLEWIGYIQYSGSTNYNPSLKSRVTISRDTSKNQFSLKLSSVTAADTAVYYCARSVNYYGNSFAVGYWGQGTLVTVSS

SEQ ID NO: 531 - protein, 6 aa, synthetic construct
  REGION 1..6: note = Recombinant protein
SDSAWS

SEQ ID NO: 532 - protein, 16 aa, synthetic construct
  REGION 1..16: note = Recombinant protein
YIQYSGSTNYNPSLKS

SEQ ID NO: 533 - protein, 13 aa, synthetic construct
  REGION 1..13: note = Recombinant protein
SVNYYGNSFAVGY

SEQ ID NO: 534 - protein, 107 aa, synthetic construct; identical to SEQ ID NO: 310
  REGION 1..107: note = Recombinant protein

SEQ ID NO: 535 - protein, 11 aa, synthetic construct
  REGION 1..11: note = Recombinant protein
RASQGISDYLA

SEQ ID NO: 536 - protein, 7 aa, synthetic construct
  REGION 1..7: note = Recombinant protein
YASQPAT

SEQ ID NO: 537 - protein, 9 aa, synthetic construct
  REGION 1..9: note = Recombinant protein
QNGHSFPLT

SEQ ID NO: 538 - protein, 122 aa, synthetic construct
  REGION 1..122: note = Recombinant protein
QVQLQESGPGLVEPSQTLSLTCTVSGYSITSDSAWSWIRQPPGKKLEWIGYIQYSGSTNYNPSLKSRVTISRDTSKNQFSLKLSSVTAADTAVYYCARSVNYYGNSFAVGYWGQGTLVTVSS

SEQ ID NO: 539 - protein, 6 aa, synthetic construct
  REGION 1..6: note = Recombinant protein
SDSAWS

SEQ ID NO: 540 - protein, 16 aa, synthetic construct
  REGION 1..16: note = Recombinant protein
YIQYSGSTNYNPSLKS

SEQ ID NO: 541 - protein, 13 aa, synthetic construct
  REGION 1..13: note = Recombinant protein
SVNYYGNSFAVGY

SEQ ID NO: 542 - protein, 107 aa, synthetic construct; identical to SEQ ID NO: 310
  REGION 1..107: note = Recombinant protein

SEQ ID NO: 543 - protein, 11 aa, synthetic construct
  REGION 1..11: note = Recombinant protein
RASQGISDYLA

SEQ ID NO: 544 - protein, 7 aa, synthetic construct
  REGION 1..7: note = Recombinant protein
YASQPAT

SEQ ID NO: 545 - protein, 9 aa, synthetic construct
  REGION 1..9: note = Recombinant protein
QNGHSFPLT

SEQ ID NO: 546 - protein, 122 aa, synthetic construct
  REGION 1..122: note = Recombinant protein
QVQLQESGPGLVEPSQTLSLTCTVSGYSITSDSAWSWIRQPPGKKLEWIGYIQYSGSTNYNPSLKSRVTISRDTSKNQFSLKLSSVTAADTAVYYCARSVNYYGNSFAVGYWGQGTSVTVSS

SEQ ID NO: 547 - protein, 6 aa, synthetic construct
  REGION 1..6: note = Recombinant protein
SDSAWS

SEQ ID NO: 548 - protein, 16 aa, synthetic construct
  REGION 1..16: note = Recombinant protein
YIQYSGSTNYNPSLKS

SEQ ID NO: 549 - protein, 13 aa, synthetic construct
  REGION 1..13: note = Recombinant protein
SVNYYGNSFAVGY

SEQ ID NO: 550 - protein, 107 aa, synthetic construct; identical to SEQ ID NO: 310
  REGION 1..107: note = Recombinant protein

SEQ ID NO: 551 - protein, 11 aa, synthetic construct
  REGION 1..11: note = Recombinant protein
RASQGISDYLA

SEQ ID NO: 552 - protein, 7 aa, synthetic construct
  REGION 1..7: note = Recombinant protein
YASQPAT

SEQ ID NO: 553 - protein, 9 aa, synthetic construct
  REGION 1..9: note = Recombinant protein
QNGHSFPLT

SEQ ID NO: 554 - protein, 122 aa, synthetic construct
  REGION 1..122: note = Recombinant protein
QVQLQESGPGLVKPSDTLSLTCAVSGYSISSDSAWSWIRQFPGKGLEWIGYIQYSGSTNYNPSLKSRVTMSVDTSKNQFSLKLSSVTALDTAVYYCARSVNYYGNSFAVGPWGQGTLVTVSS

SEQ ID NO: 555 - protein, 6 aa, synthetic construct
  REGION 1..6: note = Recombinant protein
SDSAWS

SEQ ID NO: 556 - protein, 16 aa, synthetic construct
  REGION 1..16: note = Recombinant protein
YIQYSGSTNYNPSLKS

SEQ ID NO: 557 - protein, 13 aa, synthetic construct
  REGION 1..13: note = Recombinant protein
SVNYYGNSFAVGP

SEQ ID NO: 558 - protein, 107 aa, synthetic construct; identical to SEQ ID NO: 310
  REGION 1..107: note = Recombinant protein

SEQ ID NO: 559 - protein, 11 aa, synthetic construct
  REGION 1..11: note = Recombinant protein
RASQGISDYLA

SEQ ID NO: 560 - protein, 7 aa, synthetic construct
  REGION 1..7: note = Recombinant protein
YASQPAT

SEQ ID NO: 561 - protein, 9 aa, synthetic construct
  REGION 1..9: note = Recombinant protein
QNGHSFPLT

SEQ ID NO: 562 - protein, 122 aa, synthetic construct
  REGION 1..122: note = Recombinant protein
QVQLQESGPGLVKPSDTLSLTCAVSGYSISSDSAWSWIRQPPGKGLEWIGYIQYSGSTNYNPSLKSRVTMSRDTSKNQFSLKLSSVTALDTAVYYCARSVNYYGNSFAVGPWGQGTLVTVSS

SEQ ID NO: 563 - protein, 6 aa, synthetic construct
  REGION 1..6: note = Recombinant protein
SDSAWS

SEQ ID NO: 564 - protein, 16 aa, synthetic construct
  REGION 1..16: note = Recombinant protein
YIQYSGSTNYNPSLKS

SEQ ID NO: 565 - protein, 13 aa, synthetic construct
  REGION 1..13: note = Recombinant protein
SVNYYGNSFAVGP

SEQ ID NO: 566 - protein, 107 aa, synthetic construct; identical to SEQ ID NO: 310
  REGION 1..107: note = Recombinant protein

SEQ ID NO: 567 - protein, 11 aa, synthetic construct
  REGION 1..11: note = Recombinant protein
RASQGISDYLA

SEQ ID NO: 568 - protein, 7 aa, synthetic construct
  REGION 1..7: note = Recombinant protein
YASQPAT

SEQ ID NO: 569 - protein, 9 aa, synthetic construct
  REGION 1..9: note = Recombinant protein
QNGHSFPLT

SEQ ID NO: 570 - protein, 122 aa, synthetic construct
  REGION 1..122: note = Recombinant protein
QVQLQESGPGLVKPSDTLSLTCAVSGYSISSDSAWSWIRQPPGKGLEWIGYIQYSGSTNYNPSLKSRVTMSRDTSKNQFSLKLSSVTAADTAVYYCARSVNYYGNSFAVGPWGQGTLVTVSS

SEQ ID NO: 571 - protein, 6 aa, synthetic construct
  REGION 1..6: note = Recombinant protein
SDSAWS

SEQ ID NO: 572 - protein, 16 aa, synthetic construct
  REGION 1..16: note = Recombinant protein
YIQYSGSTNYNPSLKS

SEQ ID NO: 573 - protein, 13 aa, synthetic construct
  REGION 1..13: note = Recombinant protein
SVNYYGNSFAVGP

SEQ ID NO: 574 - protein, 107 aa, synthetic construct; identical to SEQ ID NO: 310
  REGION 1..107: note = Recombinant protein

SEQ ID NO: 575 - protein, 11 aa, synthetic construct
  REGION 1..11: note = Recombinant protein
RASQGISDYLA

SEQ ID NO: 576 - protein, 7 aa, synthetic construct
  REGION 1..7: note = Recombinant protein
YASQPAT

SEQ ID NO: 577 - protein, 9 aa, synthetic construct
  REGION 1..9: note = Recombinant protein
QNGHSFPLT

SEQ ID NO: 578 - protein, 122 aa, synthetic construct
  REGION 1..122: note = Recombinant protein
QVQLQESGPGLVKPSDTLSLTCAVSGYSISSDSAWSWIRQPPGKGLEWIGYIQYSGSTNYNPSLKSRVTMSVDTSKNQFSLKLSSVTALDTAVYYCARSVNYYGNSFAVGPWGQGTLVTVSS

SEQ ID NO: 579 - protein, 6 aa, synthetic construct
  REGION 1..6: note = Recombinant protein
SDSAWS

SEQ ID NO: 580 - protein, 16 aa, synthetic construct
  REGION 1..16: note = Recombinant protein
YIQYSGSTNYNPSLKS

SEQ ID NO: 581 - protein, 13 aa, synthetic construct
  REGION 1..13: note = Recombinant protein
SVNYYGNSFAVGP

SEQ ID NO: 582 - protein, 107 aa, synthetic construct; identical to SEQ ID NO: 310
  REGION 1..107: note = Recombinant protein

SEQ ID NO: 583 - protein, 11 aa, synthetic construct
  REGION 1..11: note = Recombinant protein
RASQGISDYLA

SEQ ID NO: 584 - protein, 7 aa, synthetic construct
  REGION 1..7: note = Recombinant protein
YASQPAT

SEQ ID NO: 585 - protein, 9 aa, synthetic construct
  REGION 1..9: note = Recombinant protein
QNGHSFPLT

SEQ ID NO: 586 - protein, 122 aa, synthetic construct
  REGION 1..122: note = Recombinant protein
QVQLQESGPGLVKPSDTLSLTCAVSGYSISSDSAWSWIRQPPGKGLEWIGYIQYSGSTNYNPSLKSRVTMSVDTSKNQFSLKLSSVTALDTAVYYCARSVNYYGNSFAVGYWGQGTLVTVSS

SEQ ID NO: 587 - protein, 6 aa, synthetic construct
  REGION 1..6: note = Recombinant protein
SDSAWS

SEQ ID NO: 588 - protein, 16 aa, synthetic construct
  REGION 1..16: note = Recombinant protein
YIQYSGSTNYNPSLKS

SEQ ID NO: 589 - protein, 13 aa, synthetic construct
  REGION 1..13: note = Recombinant protein
SVNYYGNSFAVGY

SEQ ID NO: 590 - protein, 107 aa, synthetic construct; identical to SEQ ID NO: 310
  REGION 1..107: note = Recombinant protein

SEQ ID NO: 591 - protein, 11 aa, synthetic construct
  REGION 1..11: note = Recombinant protein
RASQGISDYLA

SEQ ID NO: 592 - protein, 7 aa, synthetic construct
  REGION 1..7: note = Recombinant protein
YASQPAT

SEQ ID NO: 593 - protein, 9 aa, synthetic construct
  REGION 1..9: note = Recombinant protein
QNGHSFPLT

SEQ ID NO: 594 - protein, 122 aa, synthetic construct
  REGION 1..122: note = Recombinant protein
QVQLQESGPGLVKPSDTLSLTCAVSGYSISSDSAWSWIRQPPGKKLEWIGYIQYSGSTNYNPSLKSRVTMSVDTSKNQFSLKLSSVTALDTAVYYCARSVNYYGNSFAVGPWGQGTLVTVSS

SEQ ID NO: 595 - protein, 6 aa, synthetic construct
  REGION 1..6: note = Recombinant protein
SDSAWS

SEQ ID NO: 596 - protein, 16 aa, synthetic construct
  REGION 1..16: note = Recombinant protein
YIQYSGSTNYNPSLKS

SEQ ID NO: 597 - protein, 13 aa, synthetic construct
  REGION 1..13: note = Recombinant protein
SVNYYGNSFAVGP

SEQ ID NO: 598 - protein, 107 aa, synthetic construct; identical to SEQ ID NO: 310
  REGION 1..107: note = Recombinant protein

SEQ ID NO: 599 - protein, 11 aa, synthetic construct
  REGION 1..11: note = Recombinant protein
RASQGISDYLA

SEQ ID NO: 600 - protein, 7 aa, synthetic construct
  REGION 1..7: note = Recombinant protein
YASQPAT

SEQ ID NO: 601 - protein, 9 aa, synthetic construct
  REGION 1..9: note = Recombinant protein
QNGHSFPLT

SEQ ID NO: 602 - protein, 122 aa, synthetic construct
  REGION 1..122: note = Recombinant protein
QVQLQESGPGLVKPSQTLSLTCTVSGGSISSDSAWSWIRQPPGKGLEWIGYIQYSGSTNYNPSLKSRVTISVDTSKNQFSLKLSSVTAADTAVYYCARSVNYYGNSFAVGYWGQGTLVTVSS

SEQ ID NO: 603 - protein, 6 aa, synthetic construct
  REGION 1..6: note = Recombinant protein
SDSAWS

SEQ ID NO: 604 - protein, 16 aa, synthetic construct
  REGION 1..16: note = Recombinant protein
YIQYSGSTNYNPSLKS

SEQ ID NO: 605 - protein, 13 aa, synthetic construct
  REGION 1..13: note = Recombinant protein
SVNYYGNSFAVGY

SEQ ID NO: 606 - protein, 107 aa, synthetic construct; identical to SEQ ID NO: 310
  REGION 1..107: note = Recombinant protein

SEQ ID NO: 607 - protein, 11 aa, synthetic construct
  REGION 1..11: note = Recombinant protein
RASQGISDYLA

SEQ ID NO: 608 - protein, 7 aa, synthetic construct
  REGION 1..7: note = Recombinant protein
YASQPAT

SEQ ID NO: 609 - protein, 9 aa, synthetic construct
  REGION 1..9: note = Recombinant protein
QNGHSFPLT

SEQ ID NO: 610 - protein, 122 aa, synthetic construct
  REGION 1..122: note = Recombinant protein
QVQLQESGPGLVKPSQTLSLTCTVSGGSITSDSAWSWIRQPPGKGLEWIGYIQYSGSTNYNPSLKSRVTISVDTSKNQFSLKLSSVTAADTAVYYCARSVNYYGNSFAVGYWGQGTLVTVSS

SEQ ID NO: 611 - protein, 6 aa, synthetic construct
  REGION 1..6: note = Recombinant protein
SDSAWS

SEQ ID NO: 612 - protein, 16 aa, synthetic construct
  REGION 1..16: note = Recombinant protein
YIQYSGSTNYNPSLKS

SEQ ID NO: 613 - protein, 13 aa, synthetic construct
  REGION 1..13: note = Recombinant protein
SVNYYGNSFAVGY

SEQ ID NO: 614 - protein, 107 aa, synthetic construct; identical to SEQ ID NO: 310
  REGION 1..107: note = Recombinant protein

SEQ ID NO: 615 - protein, 11 aa, synthetic construct
  REGION 1..11: note = Recombinant protein
RASQGISDYLA

SEQ ID NO: 616 - protein, 7 aa, synthetic construct
  REGION 1..7: note = Recombinant protein
YASQPAT

SEQ ID NO: 617 - protein, 9 aa, synthetic construct
  REGION 1..9: note = Recombinant protein
QNGHSFPLT

SEQ ID NO: 618 - protein, 122 aa, synthetic construct
  REGION 1..122: note = Recombinant protein
QVQLQESGPGLVKPSQTLSLTCTVSGYSISSDSAWSWIRQFPGKGLEWIGYIQYSGSTNYNPSLKSRVTISRDTSKNQFSLKLSSVTAADTAVYYCARSVNYYGNSFAVGPWGQGTLVTVSS

SEQ ID NO: 619 - protein, 6 aa, synthetic construct
  REGION 1..6: note = Recombinant protein
SDSAWS

SEQ ID NO: 620 - protein, 16 aa, synthetic construct
  REGION 1..16: note = Recombinant protein
YIQYSGSTNYNPSLKS

SEQ ID NO: 621 - protein, 13 aa, synthetic construct
  REGION 1..13: note = Recombinant protein
SVNYYGNSFAVGP

SEQ ID NO: 622 - protein, 107 aa, synthetic construct; identical to SEQ ID NO: 310
  REGION 1..107: note = Recombinant protein

SEQ ID NO: 623 - protein, 11 aa, synthetic construct
  REGION 1..11: note = Recombinant protein
RASQGISDYLA

SEQ ID NO: 624 - protein, 7 aa, synthetic construct
  REGION 1..7: note = Recombinant protein
YASQPAT

SEQ ID NO: 625 - protein, 9 aa, synthetic construct
  REGION 1..9: note = Recombinant protein
QNGHSFPLT

SEQ ID NO: 626 - protein, 122 aa, synthetic construct
  REGION 1..122: note = Recombinant protein
QVQLQESGPGLVKPSQTLSLTCTVSGYSISSDSAWSWIRQPPGKGLEWIGYIQYSGSTNYNPSLKSRVTISRDTSKNQFSLKLSSVTAADTAVYYCARSVNYYGNSFAVGPWGQGTLVTVSS

SEQ ID NO: 627 - protein, 6 aa, synthetic construct
  REGION 1..6: note = Recombinant protein
SDSAWS

SEQ ID NO: 628 - protein, 16 aa, synthetic construct
  REGION 1..16: note = Recombinant protein
YIQYSGSTNYNPSLKS

SEQ ID NO: 629 - protein, 13 aa, synthetic construct
  REGION 1..13: note = Recombinant protein
SVNYYGNSFAVGP

SEQ ID NO: 630 - protein, 107 aa, synthetic construct; identical to SEQ ID NO: 310
  REGION 1..107: note = Recombinant protein

SEQ ID NO: 631 - protein, 11 aa, synthetic construct
  REGION 1..11: note = Recombinant protein
RASQGISDYLA

SEQ ID NO: 632 - protein, 7 aa, synthetic construct
  REGION 1..7: note = Recombinant protein
YASQPAT

SEQ ID NO: 633 - protein, 9 aa, synthetic construct
  REGION 1..9: note = Recombinant protein
QNGHSFPLT

SEQ ID NO: 634 - protein, 122 aa, synthetic construct
  REGION 1..122: note = Recombinant protein
QVQLQESGPGLVKPSQTLSLTCTVSGYSISSDSAWSWIRQPPGKGLEWIGYIQYSGSTNYNPSLKSRVTISRDTSKNQFSLKLSSVTAADTAVYYCARSVNYYGNSFAVGYWGQGTLVTVSS

SEQ ID NO: 635 - protein, 6 aa, synthetic construct
  REGION 1..6: note = Recombinant protein
SDSAWS

SEQ ID NO: 636 - protein, 16 aa, synthetic construct
  REGION 1..16: note = Recombinant protein
YIQYSGSTNYNPSLKS

SEQ ID NO: 637 - protein, 13 aa, synthetic construct
  REGION 1..13: note = Recombinant protein
SVNYYGNSFAVGY

SEQ ID NO: 638 - protein, 107 aa, synthetic construct; identical to SEQ ID NO: 310
  REGION 1..107: note = Recombinant protein

SEQ ID NO: 639 - protein, 11 aa, synthetic construct
  REGION 1..11: note = Recombinant protein
RASQGISDYLA

SEQ ID NO: 640 - protein, 7 aa, synthetic construct
  REGION 1..7: note = Recombinant protein
YASQPAT

SEQ ID NO: 641 - protein, 9 aa, synthetic construct
  REGION 1..9: note = Recombinant protein
QNGHSFPLT

SEQ ID NO: 642 - protein, 122 aa, synthetic construct
  REGION 1..122: note = Recombinant protein
QVQLQESGPGLVKPSQTLSLTCTVSGYSISSDSAWSWIRQPPGKGLEWIGYIYYSGSTNYNPSLKSRVTISVDTSKNQFSLKLSSVTAADTAVYYCARSVNYYGNSFAVGYWGQGTSVTVSS

SEQ ID NO: 643 - protein, 6 aa, synthetic construct
  REGION 1..6: note = Recombinant protein
SDSAWS

SEQ ID NO: 644 - protein, 16 aa, synthetic construct
  REGION 1..16: note = Recombinant protein
YIYYSGSTNYNPSLKS

SEQ ID NO: 645 - protein, 13 aa, synthetic construct
  REGION 1..13: note = Recombinant protein
SVNYYGNSFAVGY

SEQ ID NO: 646 - protein, 107 aa, synthetic construct; identical to SEQ ID NO: 310
  REGION 1..107: note = Recombinant protein

SEQ ID NO: 647 - protein, 11 aa, synthetic construct
  REGION 1..11: note = Recombinant protein
RASQGISDYLA

SEQ ID NO: 648 - protein, 7 aa, synthetic construct
  REGION 1..7: note = Recombinant protein
YASQPAT

SEQ ID NO: 649 - protein, 9 aa, synthetic construct
  REGION 1..9: note = Recombinant protein
QNGHSFPLT

SEQ ID NO: 650 - protein, 122 aa, synthetic construct
  REGION 1..122: note = Recombinant protein
QVQLQESGPGLVKPSQTLSLTCTVSGYSISSDSAWSWIRQPPGKKLEWIGYIQYSGSTNYNPSLKSRVTISRDTSKNQFSLKLSSVTAADTAVYYCARSVNYYGNSFAVGPWGQGTLVTVSS

SEQ ID NO: 651 - protein, 6 aa, synthetic construct
  REGION 1..6: note = Recombinant protein
SDSAWS

SEQ ID NO: 652 - protein, 16 aa, synthetic construct
  REGION 1..16: note = Recombinant protein
YIQYSGSTNYNPSLKS

SEQ ID NO: 653 - protein, 13 aa, synthetic construct
  REGION 1..13: note = Recombinant protein
SVNYYGNSFAVGP

SEQ ID NO: 654 - protein, 107 aa, synthetic construct; identical to SEQ ID NO: 318
  REGION 1..107: note = Recombinant protein

SEQ ID NO: 655 - protein, 11 aa, synthetic construct
  REGION 1..11: note = Recombinant protein
RASQGISDYLH

SEQ ID NO: 656 - protein, 7 aa, synthetic construct
  REGION 1..7: note = Recombinant protein
YTSQPAT

SEQ ID NO: 657 - protein, 9 aa, synthetic construct
  REGION 1..9: note = Recombinant protein
QNGHSFPLT

SEQ ID NO: 658 - protein, 122 aa, synthetic construct; identical to SEQ ID NO: 634
  REGION 1..122: note = Recombinant protein

SEQ ID NO: 659 - protein, 6 aa, synthetic construct
  REGION 1..6: note = Recombinant protein
SDSAWS

SEQ ID NO: 660 - protein, 16 aa, synthetic construct
  REGION 1..16: note = Recombinant protein
YIQYSGSTNYNPSLKS

SEQ ID NO: 661 - protein, 13 aa, synthetic construct
  REGION 1..13: note = Recombinant protein
SVNYYGNSFAVGY

SEQ ID NO: 662 - protein, 107 aa, synthetic construct; identical to SEQ ID NO: 358
  REGION 1..107: note = Recombinant protein

SEQ ID NO: 663 - protein, 11 aa, synthetic construct
  REGION 1..11: note = Recombinant protein
RASQGISDYLH

SEQ ID NO: 664 - protein, 7 aa, synthetic construct
  REGION 1..7: note = Recombinant protein
YTSQPAT

SEQ ID NO: 665 - protein, 9 aa, synthetic construct
  REGION 1..9: note = Recombinant protein
QNGHSFPLT

SEQ ID NO: 666 - protein, 122 aa, synthetic construct; identical to SEQ ID NO: 530
  REGION 1..122: note = Recombinant protein

SEQ ID NO: 667 - protein, 6 aa, synthetic construct
  REGION 1..6: note = Recombinant protein
SDSAWS

SEQ ID NO: 668 - protein, 16 aa, synthetic construct
  REGION 1..16: note = Recombinant protein
YIQYSGSTNYNPSLKS

SEQ ID NO: 669 - protein, 13 aa, synthetic construct
  REGION 1..13: note = Recombinant protein
SVNYYGNSFAVGY

SEQ ID NO: 670 - protein, 107 aa, synthetic construct; identical to SEQ ID NO: 318
  REGION 1..107: note = Recombinant protein

SEQ ID NO: 671 - protein, 11 aa, synthetic construct
  REGION 1..11: note = Recombinant protein
RASQGISDYLH

SEQ ID NO: 672 - protein, 7 aa, synthetic construct
  REGION 1..7: note = Recombinant protein
YTSQPAT

SEQ ID NO: 673 - protein, 9 aa, synthetic construct
  REGION 1..9: note = Recombinant protein
QNGHSFPLT

SEQ ID NO: 674 - protein, 122 aa, synthetic construct; identical to SEQ ID NO: 522
  REGION 1..122: note = Recombinant protein

SEQ ID NO: 675 - protein, 6 aa, synthetic construct
  REGION 1..6: note = Recombinant protein
SDSAWS

SEQ ID NO: 676 - protein, 16 aa, synthetic construct
  REGION 1..16: note = Recombinant protein
YIQYSGSTNYNPSLKS

SEQ ID NO: 677 - protein, 13 aa, synthetic construct
  REGION 1..13: note = Recombinant protein
SVNYYGNSFAVGP

SEQ ID NO: 678 - protein, 107 aa, synthetic construct; identical to SEQ ID NO: 318
  REGION 1..107: note = Recombinant protein

SEQ ID NO: 679 - protein, 11 aa, synthetic construct
  REGION 1..11: note = Recombinant protein
RASQGISDYLH

SEQ ID NO: 680 - protein, 7 aa, synthetic construct
  REGION 1..7: note = Recombinant protein
YTSQPAT

SEQ ID NO: 681 - protein, 9 aa, synthetic construct
  REGION 1..9: note = Recombinant protein
QNGHSFPLT

SEQ ID NO: 682 - protein, 122 aa, synthetic construct; identical to SEQ ID NO: 626
  REGION 1..122: note = Recombinant protein

SEQ ID NO: 683 - protein, 6 aa, synthetic construct
  REGION 1..6: note = Recombinant protein
SDSAWS

SEQ ID NO: 684 - protein, 16 aa, synthetic construct
  REGION 1..16: note = Recombinant protein
YIQYSGSTNYNPSLKS

SEQ ID NO: 685 - protein, 13 aa, synthetic construct
  REGION 1..13: note = Recombinant protein
SVNYYGNSFAVGP

SEQ ID NO: 686 - protein, 107 aa, synthetic construct; identical to SEQ ID NO: 358
  REGION 1..107: note = Recombinant protein

SEQ ID NO: 687 - protein, 11 aa, synthetic construct
  REGION 1..11: note = Recombinant protein
RASQGISDYLH

SEQ ID NO: 688 - protein, 7 aa, synthetic construct
  REGION 1..7: note = Recombinant protein
YTSQPAT

SEQ ID NO: 689 - protein, 9 aa, synthetic construct
  REGION 1..9: note = Recombinant protein
QNGHSFPLT

SEQ ID NO: 690 - protein, 122 aa, synthetic construct; identical to SEQ ID NO: 618
  REGION 1..122: note = Recombinant protein

SEQ ID NO: 691 - protein, 6 aa, synthetic construct
  REGION 1..6: note = Recombinant protein
SDSAWS

SEQ ID NO: 692 - protein, 16 aa, synthetic construct
  REGION 1..16: note = Recombinant protein
YIQYSGSTNYNPSLKS

SEQ ID NO: 693 - protein, 13 aa, synthetic construct
  REGION 1..13: note = Recombinant protein
SVNYYGNSFAVGP

SEQ ID NO: 694 - protein, 107 aa, Mus musculus
DIVMSQSPATLSVTPGDRVSLSCRTSQDIGDYLHWYRQKSHESPRLLIKYTSQSVSGIPSRFSGSGSGSDFTLSINTVEPEDVGLYYCQNGHSFPLTFGTGTRLELK

SEQ ID NO: 695 - protein, 11 aa, Mus musculus
RTSQDIGDYLH

SEQ ID NO: 696 - protein, 7 aa, Mus musculus
YTSQSVS

SEQ ID NO: 697 - protein, 9 aa, Mus musculus
QNGHSFPLT

SEQ ID NO: 698 - protein, 122 aa, Mus musculus
DVQLQESGPGLVKPSQSLSLTCTVTGYSITSDCAWNWIRQFPGNKLEWMGYINYSGSTNYNPSLKSRISITRDTSQNHFFLQLSSVTAEDTATYYCTRSVNYYGNSFAVGYWGQGISVTVSS

SEQ ID NO: 699 - protein, 6 aa, Mus musculus
SDCAWN

SEQ ID NO: 700 - protein, 16 aa, Mus musculus
YINYSGSTNYNPSLKS

SEQ ID NO: 701 - protein, 13 aa, Mus musculus
SVNYYGNSFAVGY

SEQ ID NO: 702 - protein, 107 aa, Mus musculus
DIVMTQSPATLSVTPGDRVSLSCRASQSISDYLHWYQQKSHESPRLLIKYSSQSISGIPSRFSGSGSGSDFTLSINSVEPEDVGVYYCQNGHSFPLTFGAGTKLELK

SEQ ID NO: 703 - protein, 11 aa, Mus musculus
RASQSISDYLH

SEQ ID NO: 704 - protein, 7 aa, Mus musculus
YSSQSIS

SEQ ID NO: 705 - protein, 9 aa, Mus musculus
QNGHSFPLT

SEQ ID NO: 706 - protein, 122 aa, Mus musculus
DVQLQESGPGLVKPSQSLSLTCTVTGYSITSDCAWNWIRQFPGNKLEWMGYINYSGSTNYNPSLKSRISITRDTSKNHFFLQLNSVTAEDTATYYCARSVDYFGNSFAVGYWGQGTSVAVSS

SEQ ID NO: 707 - protein, 6 aa, Mus musculus
SDCAWN

SEQ ID NO: 708 - protein, 16 aa, Mus musculus
YINYSGSTNYNPSLKS

SEQ ID NO: 709 - protein, 13 aa, Mus musculus
SVDYFGNSFAVGY

SEQ ID NO: 710 - protein, 112 aa, Mus musculus
DVMVTQTPLSLPVNLGDQASISCRSSQSLVHSNGNTYLHWYLQKPGQSPKLLIYRVSDRFSGVPDRFSGSGSGTDFTLKISRVEAEDLGVYFCSQSTHVPPTFGGGTKLEIK

SEQ ID NO: 711 - protein, 16 aa, Mus musculus
RSSQSLVHSNGNTYLH

SEQ ID NO: 712 - protein, 7 aa, Mus musculus
RVSDRFS

SEQ ID NO: 713 - protein, 9 aa, Mus musculus
SQSTHVPPT

SEQ ID NO: 714 - protein, 118 aa, Mus musculus
EIQLQQTGPELVKPGASVKISCKASGYSFTDYIMVWMKQSQGKTLEWIGKINPYYGDISYNLKFKGKATLTVDKSSSTVYMQLNSLASEDSAVYYCARGFYYDYFDQWGQGTTLTVSS

SEQ ID NO: 715 - protein, 5 aa, Mus musculus
DYIMV

SEQ ID NO: 716 - protein, 17 aa, Mus musculus
KINPYYGDISYNLKFKG

SEQ ID NO: 717 - protein, 9 aa, Mus musculus
GFYYDYFDQ

SEQ ID NO: 718 - protein, 107 aa, Mus musculus
DIVMTQSPATLSVTPGDRVSLSCRASQSISDYLHWYQHKSHESPRLLIKYVSQPISGIPSRFSGSGSGSEFTLSINSVEPEDVGVYYCQNGHSFPLTFGAGTKLELK

SEQ ID NO: 719 - protein, 11 aa, Mus musculus
RASQSISDYLH

SEQ ID NO: 720 - protein, 7 aa, Mus musculus
YVSQPIS

SEQ ID NO: 721 - protein, 9 aa, Mus musculus
QNGHSFPLT

SEQ ID NO: 722 - protein, 122 aa, Mus musculus
DVQLQESGPGLVEPSQSLSLTCTVTGYSITSDCAWSWIRQFPGNKLEWMGYINYSGSTNYNPSLKSRISITRDTSKNQFFLRLNSVTGEDTATYYCARSVNYYGNSFAVGYWGQGTSVTVSS

SEQ ID NO: 723 - protein, 6 aa, Mus musculus
SDCAWS

SEQ ID NO: 724 - protein, 16 aa, Mus musculus
YINYSGSTNYNPSLKS

SEQ ID NO: 725 - protein, 13 aa, Mus musculus
SVNYYGNSFAVGY

SEQ ID NO: 726 - protein, 107 aa, Mus musculus
DILLTQSPAILSVSPGERVSFSCRASQSIGTSIHWYQQRTNGSPRLLIKYASESISGIPSRFSGSGSGTDFTLSINSVESEDIADYYCQQSNSWPFTFGSGTKLEIK

SEQ ID NO: 727 - protein, 11 aa, Mus musculus
RASQSIGTSIH

SEQ ID NO: 728 - protein, 7 aa, Mus musculus
YASESIS

SEQ ID NO: 729 - protein, 9 aa, Mus musculus
QQSNSWPFT

SEQ ID NO: 730 - protein, 119 aa, Mus musculus
QVQLQQSGAELMKPGASVKISCKATGYTFNTYWIEWVKQRPGHGLEWIGEILPGRGTTNYNENFKGKATITADISSNTAYLQLSSLTGEDSAVYYCARLTPLSLRTGYWGQGTTLTVSS

SEQ ID NO: 731 - protein, 5 aa, Mus musculus
TYWIE

SEQ ID NO: 732 - protein, 17 aa, Mus musculus
EILPGRGTTNYNENFKG

SEQ ID NO: 733 - protein, 10 aa, Mus musculus
LTPLSLRTGY

SEQ ID NO: 734 - protein, 111 aa, Mus musculus
DIVLTQSPPSLAVSLGQRAIISCKASQSVSFAGTSLMYWYQQKPGQQPKVLMYRASNLEAGVPIRFSGSGSGTDFTLNIHPVEEDDVATYYCQQSREYPYTFGGGTKLEIK

SEQ ID NO: 735 - protein, 15 aa, synthetic construct
  REGION 1..15: note = Recombinant protein
KASQSVSFAGTSLMY

SEQ ID NO: 736 - protein, 7 aa, Mus musculus
RASNLEA

SEQ ID NO: 737 - protein, 9 aa, Mus musculus
QQSREYPYT

SEQ ID NO: 738 - protein, 118 aa, Mus musculus
QIQLQQSGPELVKPGTSVKMACKASDYTFTDYYLHWVKQRPGQGLEWIGWIYPGSGNTKFNEKFKGKATLTVDTSSSTAYMHLSSLTSDDTAVYFCTRVLGRGAMDYWGQGTSVTVSS

SEQ ID NO: 739 - protein, 5 aa, Mus musculus
DYYLH

SEQ ID NO: 740 - protein, 17 aa, Mus musculus
WIYPGSGNTKFNEKFKG

SEQ ID NO: 741 - protein, 9 aa, Mus musculus
VLGRGAMDY

SEQ ID NO: 742 - protein, 106 aa, Mus musculus
QIVLTQSPAIMSASPGEKVTITCSASSSVSYMFWFQQKPGTSPKLWIYSTSNLASGVPARFSGSGSGTSYSLTISRMEAEDAATYYCQQRSSYPLTFGAGTKLELK

SEQ ID NO: 743 - protein, 10 aa, Mus musculus
SASSSVSYMF

SEQ ID NO: 744 - protein, 7 aa, Mus musculus
STSNLAS

SEQ ID NO: 745 - protein, 9 aa, Mus musculus
QQRSSYPLT

SEQ ID NO: 746 - protein, 118 aa, Mus musculus
QVQLQQSGAELLKPGASVKISCKATDYTFSRYWIEWVKQRPGHGLEWIGEILPGSVNIKYNEIFKGKATFTAETSSNTAYMQLSSLTSEDSAVYYCTRTPYLQRGAYWGQGTLVTVSA

SEQ ID NO: 747 - protein, 5 aa, Mus musculus
RYWIE

SEQ ID NO: 748 - protein, 17 aa, Mus musculus
EILPGSVNIKYNEIFKG

SEQ ID NO: 749 - protein, 9 aa, Mus musculus
TPYLQRGAY

SEQ ID NO: 750 - protein, 107 aa, Mus musculus; identical to SEQ ID NO: 22

SEQ ID NO: 751 - protein, 11 aa, Mus musculus
RASQGISDYLH

SEQ ID NO: 752 - protein, 7 aa, Mus musculus
YTSQPVS

SEQ ID NO: 753 - protein, 9 aa, Mus musculus
QNGHSFPLT

SEQ ID NO: 754 - protein, 122 aa, Mus musculus; identical to SEQ ID NO: 26

SEQ ID NO: 755 - protein, 6 aa, Mus musculus
SDCAWS

SEQ ID NO: 756 - protein, 16 aa, Mus musculus
YINYSGSTNYNPSLKS

SEQ ID NO: 757 - protein, 13 aa, Mus musculus
SVNYYGNSFAVGY

SEQ ID NO: 758 - protein, 107 aa, synthetic construct; identical to SEQ ID NO: 318
  REGION 1..107: note = Recombinant protein

SEQ ID NO: 759 - protein, 11 aa, synthetic construct
  REGION 1..11: note = Recombinant protein
RASQGISDYLH

SEQ ID NO: 760 - protein, 7 aa, synthetic construct
  REGION 1..7: note = Recombinant protein
YTSQPAT

SEQ ID NO: 761 - protein, 9 aa, synthetic construct
  REGION 1..9: note = Recombinant protein
QNGHSFPLT

SEQ ID NO: 762 - protein, 122 aa, synthetic construct; identical to SEQ ID NO: 634
  REGION 1..122: note = Recombinant protein

SEQ ID NO: 763 - protein, 6 aa, synthetic construct
  REGION 1..6: note = Recombinant protein
SDSAWS

SEQ ID NO: 764 - protein, 16 aa, synthetic construct
  REGION 1..16: note = Recombinant protein
YIQYSGSTNYNPSLKS

SEQ ID NO: 765 - protein, 13 aa, synthetic construct
  REGION 1..13: note = Recombinant protein
SVNYYGNSFAVGY

SEQ ID NO: 766 - protein, 107 aa, synthetic construct; identical to SEQ ID NO: 14
  REGION 1..107: note = Recombinant protein

SEQ ID NO: 767 - protein, 11 aa, synthetic construct
  REGION 1..11: note = Recombinant protein
KASQDVDPAVA

SEQ ID NO: 768 - protein, 7 aa, synthetic construct
  REGION 1..7: note = Recombinant protein
WASTRHT

SEQ ID NO: 769 - protein, 9 aa, synthetic construct
  REGION 1..9: note = Recombinant protein
QQYSKYPYT

SEQ ID NO: 770 - protein, 118 aa, synthetic construct; identical to SEQ ID NO: 18
  REGION 1..118: note = Recombinant protein

SEQ ID NO: 771 - protein, 5 aa, synthetic construct
  REGION 1..5: note = Recombinant protein
DYGVS

SEQ ID NO: 772 - protein, 16 aa, synthetic construct
  REGION 1..16: note = Recombinant protein
VIWGGGNTYNNSALKS

SEQ ID NO: 773 - protein, 10 aa, synthetic construct
  REGION 1..10: note = Recombinant protein
HGYYGYVMDY

SEQ ID NO: 774 - protein, 107 aa, synthetic construct; identical to SEQ ID NO: 318
  REGION 1..107: note = Recombinant protein

SEQ ID NO: 775 - protein, 11 aa, synthetic construct
  REGION 1..11: note = Recombinant protein
RASQGISDYLH

SEQ ID NO: 776 - protein, 7 aa, synthetic construct
  REGION 1..7: note = Recombinant protein
YTSQPAT

SEQ ID NO: 777 - protein, 9 aa, synthetic construct
  REGION 1..9: note = Recombinant protein
QNGHSFPLT

SEQ ID NO: 778 - protein, 122 aa, synthetic construct; identical to SEQ ID NO: 634
  REGION 1..122: note = Recombinant protein

SEQ ID NO: 779 - protein, 6 aa, synthetic construct
  REGION 1..6: note = Recombinant protein
SDSAWS

SEQ ID NO: 780 - protein, 16 aa, synthetic construct
  REGION 1..16: note = Recombinant protein
YIQYSGSTNYNPSLKS

SEQ ID NO: 781 - protein, 13 aa, synthetic construct
  REGION 1..13: note = Recombinant protein
SVNYYGNSFAVGY

SEQ ID NO: 782 - protein, 107 aa, synthetic construct; identical to SEQ ID NO: 78
  REGION 1..107: note = Recombinant protein

SEQ ID NO: 783 - protein, 11 aa, synthetic construct
  REGION 1..11: note = Recombinant protein
RASQGISDYLH

SEQ ID NO: 784 - protein, 7 aa, synthetic construct
  REGION 1..7: note = Recombinant protein
YTSQPAT

SEQ ID NO: 785 - protein, 9 aa, synthetic construct
  REGION 1..9: note = Recombinant protein
QSGHSFPLT

SEQ ID NO: 786 - protein, 122 aa, synthetic construct; identical to SEQ ID NO: 58
  REGION 1..122: note = Recombinant protein

SEQ ID NO: 787 - protein, 6 aa, synthetic construct
  REGION 1..6: note = Recombinant protein
SDSAWS

SEQ ID NO: 788 - protein, 16 aa, synthetic construct
  REGION 1..16: note = Recombinant protein
YIQYSGSTNYNPSLKS

SEQ ID NO: 789 - protein, 13 aa, synthetic construct
  REGION 1..13: note = Recombinant protein
SVNYYGNSFAVGY

SEQ ID NO: 790 - protein, 107 aa, synthetic construct; identical to SEQ ID NO: 22
  REGION 1..107: note = Recombinant protein

SEQ ID NO: 791 - protein, 11 aa, synthetic construct
  REGION 1..11: note = Recombinant protein
RASQGISDYLH

SEQ ID NO: 792 - protein, 7 aa, synthetic construct
  REGION 1..7: note = Recombinant protein
YTSQPVS

SEQ ID NO: 793 - protein, 9 aa, synthetic construct
  REGION 1..9: note = Recombinant protein
QNGHSFPLT

SEQ ID NO: 794 - protein, 122 aa, synthetic construct; identical to SEQ ID NO: 90
  REGION 1..122: note = Recombinant protein

SEQ ID NO: 795 - protein, 6 aa, synthetic construct
  REGION 1..6: note = Recombinant protein
SDSAWS

SEQ ID NO: 796 - protein, 16 aa, synthetic construct
  REGION 1..16: note = Recombinant protein
YIQYSGSTNYNPSLKS

SEQ ID NO: 797 - protein, 13 aa, synthetic construct
  REGION 1..13: note = Recombinant protein
SVNYYGNSFWVDP

SEQ ID NO: 798 - protein, 107 aa, synthetic construct; identical to SEQ ID NO: 102
  REGION 1..107: note = Recombinant protein

SEQ ID NO: 799 - protein, 11 aa, synthetic construct
  REGION 1..11: note = Recombinant protein
RASQGISDYLH

SEQ ID NO: 800 - protein, 7 aa, synthetic construct
  REGION 1..7: note = Recombinant protein
YTSQPAT

SEQ ID NO: 801 - protein, 9 aa, synthetic construct
  REGION 1..9: note = Recombinant protein
QNGHSFPLT

SEQ ID NO: 802 - protein, 122 aa, synthetic construct; identical to SEQ ID NO: 138
  REGION 1..122: note = Recombinant protein

SEQ ID NO: 803 - protein, 6 aa, synthetic construct
  REGION 1..6: note = Recombinant protein
SDSAWS

SEQ ID NO: 804 - protein, 16 aa, synthetic construct
  REGION 1..16: note = Recombinant protein
YIQYSGSTNYNPSLKS

SEQ ID NO: 805 - protein, 13 aa, synthetic construct
  REGION 1..13: note = Recombinant protein
SVNYYGNSFAVDY

SEQ ID NO: 806 - protein, 107 aa, synthetic construct; identical to SEQ ID NO: 470
  REGION 1..107: note = Recombinant protein

SEQ ID NO: 807 - protein, 11 aa, synthetic construct
  REGION 1..11: note = Recombinant protein
RASQGVSDYLH

SEQ ID NO: 808 - protein, 7 aa, synthetic construct
  REGION 1..7: note = Recombinant protein
YASQPAT

SEQ ID NO: 809 - protein, 9 aa, synthetic construct
  REGION 1..9: note = Recombinant protein
QNGHSFPLT

SEQ ID NO: 810 - protein, 122 aa, synthetic construct; identical to SEQ ID NO: 538
  REGION 1..122: note = Recombinant protein

SEQ ID NO: 811 - protein, 6 aa, synthetic construct
  REGION 1..6: note = Recombinant protein
SDSAWS

SEQ ID NO: 812 - protein, 16 aa, synthetic construct
  REGION 1..16: note = Recombinant protein
YIQYSGSTNYNPSLKS

SEQ ID NO: 813 - protein, 13 aa, synthetic construct
  REGION 1..13: note = Recombinant protein
SVNYYGNSFAVGY

SEQ ID NO: 814 - protein, 107 aa, synthetic construct; identical to SEQ ID NO: 318
  REGION 1..107: note = Recombinant protein

SEQ ID NO: 815 - protein, 11 aa, synthetic construct
  REGION 1..11: note = Recombinant protein
RASQGISDYLH

SEQ ID NO: 816 - protein, 7 aa, synthetic construct
  REGION 1..7: note = Recombinant protein
YTSQPAT

SEQ ID NO: 817 - protein, 9 aa, synthetic construct
  REGION 1..9: note = Recombinant protein
QNGHSFPLT

SEQ ID NO: 818 - protein, 122 aa, synthetic construct; identical to SEQ ID NO: 634
  REGION 1..122: note = Recombinant protein

SEQ ID NO: 819 - protein, 6 aa, synthetic construct
  REGION 1..6: note = Recombinant protein
SDSAWS

SEQ ID NO: 820 - protein, 16 aa, synthetic construct
  REGION 1..16: note = Recombinant protein
YIQYSGSTNYNPSLKS

SEQ ID NO: 821 - protein, 13 aa, synthetic construct
  REGION 1..13: note = Recombinant protein
SVNYYGNSFAVGY

SEQ ID NO: 822 - protein, 107 aa, synthetic construct
  REGION 1..107: note = Recombinant protein
DIQMTQSPSSLSASVGDRVTITCQASQDISNYLNWYQQKPGKAPKLLIYDASNLETGVPSRFSGSGSGTDFTFTISSLQPEDIATYYCQQVAALPPTFGGGTKVEIK

SEQ ID NO: 823 - protein, 11 aa, synthetic construct
  REGION 1..11: note = Recombinant protein
QASQDISNYLN

SEQ ID NO: 824 - protein, 7 aa, synthetic construct
  REGION 1..7: note = Recombinant protein
DASNLET

SEQ ID NO: 825 - protein, 9 aa, synthetic construct
  REGION 1..9: note = Recombinant protein
QQVAALPPT

SEQ ID NO: 826 - protein, 123 aa, synthetic construct
  REGION 1..123: note = Recombinant protein
EVQLVESGGGLVKPGGSLRLSCAASGFTFTSYSMSWVRQAPGKGLEWVSSISSRSSYICYADSVKGRFTISRDNAKNSLYLQMNSLRAEDTAVYYCAREGDTGSIGGDYMDVWGKGTTVTVSS

SEQ ID NO: 827 - protein, 5 aa, synthetic construct
  REGION 1..5: note = Recombinant protein
SYSMS

SEQ ID NO: 828 - protein, 17 aa, synthetic construct
  REGION 1..17: note = Recombinant protein
SISSRSSYICYADSVKG

SEQ ID NO: 829 - protein, 14 aa, synthetic construct
  REGION 1..14: note = Recombinant protein
EGDTGSIGGDYMDV

SEQ ID NO: 830 - protein, 107 aa, synthetic construct; identical to SEQ ID NO: 318
  REGION 1..107: note = Recombinant protein

SEQ ID NO: 831 - protein, 11 aa, synthetic construct
  REGION 1..11: note = Recombinant protein
RASQGISDYLH

SEQ ID NO: 832 - protein, 7 aa, synthetic construct
  REGION 1..7: note = Recombinant protein
YTSQPAT

SEQ ID NO: 833 - protein, 9 aa, synthetic construct
  REGION 1..9: note = Recombinant protein
QNGHSFPLT

SEQ ID NO: 834 - protein, 122 aa, synthetic construct; identical to SEQ ID NO: 634
  REGION 1..122: note = Recombinant protein

SEQ ID NO: 835 - protein, 6 aa, synthetic construct
  REGION 1..6: note = Recombinant protein
SDSAWS

SEQ ID NO: 836 - protein, 16 aa, synthetic construct
  REGION 1..16: note = Recombinant protein
YIQYSGSTNYNPSLKS

SEQ ID NO: 837 - protein, 13 aa, synthetic construct
  REGION 1..13: note = Recombinant protein
SVNYYGNSFAVGY

SEQ ID NO: 838 - protein, 112 aa, synthetic construct
  REGION 1..112: note = Recombinant protein
DIVMTQTPLSSPVTLGQPASISCRSSQSLVHRQGNTYFHWLQQRPGQPPRLLIYKVSNRFSGVPDRFSGSGAGTDFTLKISRVEAEDVGVYYCSQSTHVPYTFGQGTKLEIK

SEQ ID NO: 839 - protein, 16 aa, synthetic construct
  REGION 1..16: note = Recombinant protein
RSSQSLVHRQGNTYFH

SEQ ID NO: 840 - protein, 7 aa, synthetic construct
  REGION 1..7: note = Recombinant protein
KVSNRFS

SEQ ID NO: 841 - protein, 9 aa, synthetic construct
  REGION 1..9: note = Recombinant protein
SQSTHVPYT

SEQ ID NO: 842 - protein, 114 aa, synthetic construct
  REGION 1..114: note = Recombinant protein
EVQLVESGGGLVQPGGSLRLSCAASGFTFSRYWMTWVRQAPGKGLVWVGEINPDSSTINYAPSVKGRFTISRDNAKNTLYLQMNSLRAEDTAVYYCASGVFTSWGQGTLVTVSS

SEQ ID NO: 843 - protein, 5 aa, synthetic construct
  REGION 1..5: note = Recombinant protein
RYWMT

SEQ ID NO: 844 - protein, 17 aa, synthetic construct
  REGION 1..17: note = Recombinant protein
EINPDSSTINYAPSVKG

SEQ ID NO: 845 - protein, 5 aa, synthetic construct
  REGION 1..5: note = Recombinant protein
GVFTS

SEQ ID NO: 846 - protein, 107 aa, synthetic construct; identical to SEQ ID NO: 318
  REGION 1..107: note = Recombinant protein

SEQ ID NO: 847 - protein, 11 aa, synthetic construct
  REGION 1..11: note = Recombinant protein
RASQGISDYLH

SEQ ID NO: 848 - protein, 7 aa, synthetic construct
  REGION 1..7: note = Recombinant protein
YTSQPAT

SEQ ID NO: 849 - protein, 9 aa, synthetic construct
  REGION 1..9: note = Recombinant protein
QNGHSFPLT

SEQ ID NO: 850 - protein, 122 aa, synthetic construct; identical to SEQ ID NO: 634
  REGION 1..122: note = Recombinant protein

SEQ ID NO: 851 - protein, 6 aa, synthetic construct
  REGION 1..6: note = Recombinant protein
SDSAWS

SEQ ID NO: 852 - protein, 16 aa, synthetic construct
  REGION 1..16: note = Recombinant protein
YIQYSGSTNYNPSLKS

SEQ ID NO: 853 - protein, 13 aa, synthetic construct
  REGION 1..13: note = Recombinant protein
SVNYYGNSFAVGY

SEQ ID NO: 854 - protein, 112 aa, synthetic construct; identical to SEQ ID NO: 838
  REGION 1..112: note = Recombinant protein

SEQ ID NO: 855 - protein, 16 aa, synthetic construct
  REGION 1..16: note = Recombinant protein
RSSQSLVHRQGNTYFH

SEQ ID NO: 856 - protein, 7 aa, synthetic construct
  REGION 1..7: note = Recombinant protein
KVSNRFS

SEQ ID NO: 857 - protein, 9 aa, synthetic construct
  REGION 1..9: note = Recombinant protein
SQSTHVPYT

SEQ ID NO: 858 - protein, 114 aa, synthetic construct; identical to SEQ ID NO: 842
  REGION 1..114: note = Recombinant protein

SEQ ID NO: 859 - protein, 5 aa, synthetic construct
  REGION 1..5: note = Recombinant protein
RYWMT

SEQ ID NO: 860 - protein, 17 aa, synthetic construct
  REGION 1..17: note = Recombinant protein
EINPDSSTINYAPSVKG

SEQ ID NO: 861 - protein, 5 aa, synthetic construct
  REGION 1..5: note = Recombinant protein
GVFTS

SEQ ID NO: 862 - protein, 112 aa, synthetic construct
  REGION 1..112: note = Recombinant protein
DVVMTQTPLSLPVSLGDQASISCRSSQSLVHRNGNTYFHWCLQKPGQSPKLLIYKVSNRFSGVPDRFSGSGSGTDFTLKISRVEAEDLGVYFCSQSTHVPYTFGGGTKLEIK

SEQ ID NO: 863 - protein, 16 aa, synthetic construct
  REGION 1..16: note = Recombinant protein
RSSQSLVHRNGNTYFH

SEQ ID NO: 864 - protein, 7 aa, synthetic construct
  REGION 1..7: note = Recombinant protein
KVSNRFS

SEQ ID NO: 865 - protein, 9 aa, synthetic construct
  REGION 1..9: note = Recombinant protein
SQSTHVPYT

SEQ ID NO: 866 - protein, 114 aa, synthetic construct
  REGION 1..114: note = Recombinant protein
EVKLLESGGGLVQPGGSLKLSCAASGFDFSRYWMTWVRQAPGKGLEWIGEINPDSSTINYTPSLKDKFIISRDNAKNTLYLQMSEVRSEDTALYYCASGVFTSWGQGTLVTVSA

SEQ ID NO: 867 - protein, 5 aa, synthetic construct
  REGION 1..5: note = Recombinant protein
RYWMT

SEQ ID NO: 868 - protein, 17 aa, synthetic construct
  REGION 1..17: note = Recombinant protein
EINPDSSTINYTPSLKD

SEQ ID NO: 869 - protein, 5 aa, synthetic construct
  REGION 1..5: note = Recombinant protein
GVFTS

SEQ ID NO: 870 - protein, 112 aa, synthetic construct
  REGION 1..112: note = Recombinant protein
DIVVSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLLTFGAGTKLELK

SEQ ID NO: 871 - protein, 17 aa, synthetic construct
  REGION 1..17: note = Recombinant protein
KSSQSLLNSRTRKNYLA

SEQ ID NO: 872 - protein, 7 aa, synthetic construct
  REGION 1..7: note = Recombinant protein
WASTRES

SEQ ID NO: 873 - protein, 8 aa, synthetic construct
  REGION 1..8: note = Recombinant protein
KQSYNLLT

SEQ ID NO: 874 - protein, 121 aa, synthetic construct
  REGION 1..121: note = Recombinant protein
EVRLVESGGGLVQPGGSLKLSCATSGFTFSDYFMYWIRQTPEKRLEWVAYISNGGDSSSYPDTVKGRFTISRDNAKNTLYLQMSRLKSEDTAMYYCATNKNWDDYYDMDYWGQGTSVTVSS

SEQ ID NO: 875 - protein, 5 aa, synthetic construct
  REGION 1..5: note = Recombinant protein
DYFMY

SEQ ID NO: 876 - protein, 17 aa, synthetic construct
  REGION 1..17: note = Recombinant protein
YISNGGDSSSYPDTVKG

SEQ ID NO: 877 - protein, 12 aa, synthetic construct
  REGION 1..12: note = Recombinant protein
NKNWDDYYDMDY

SEQ ID NO: 878 - protein, 107 aa, synthetic construct
  REGION 1..107: note = Recombinant protein
SIVMTQTPKFLLVSAGDRVTITCKASQSVSNDVAWYQQKPGQSPKLLINYASSRYTGIPDRFTGSGYGTDFTFTISTVQAEDLAVYFCQQDYSSPYTFGGGTKLEIE

SEQ ID NO: 879 - protein, 11 aa, synthetic construct
  REGION 1..11: note = Recombinant protein
KASQSVSNDVA

SEQ ID NO: 880 - protein, 7 aa, synthetic construct
  REGION 1..7: note = Recombinant protein
YASSRYT

SEQ ID NO: 881 - protein, 9 aa, synthetic construct
  REGION 1..9: note = Recombinant protein
QQDYSSPYT

SEQ ID NO: 882 - protein, 121 aa, synthetic construct
  REGION 1..121: note = Recombinant protein
QVQLEQSGAELVRPGVSVKISCKGSGYTFTDYSMHWVKQSHAKSLEWIGVISTYYGDVRYNQKFKGKATMTVDKSSSTAYMALARLTSEDSAIYYCARAPMITTGAWFAYWGQGTLVTVSA

SEQ ID NO: 883 - protein, 5 aa, synthetic construct
  REGION 1..5: note = Recombinant protein
DYSMH

SEQ ID NO: 884 - protein, 17 aa, synthetic construct
  REGION 1..17: note = Recombinant protein
VISTYYGDVRYNQKFKG

SEQ ID NO: 885 - protein, 12 aa, synthetic construct
  REGION 1..12: note = Recombinant protein
APMITTGAWFAY

SEQ ID NO: 886 - protein, 112 aa, synthetic construct
  REGION 1..112: note = Recombinant protein
DVVMTQTPLSLPVSLGDQASISCRSSQSLVHRNGNTYFHWALQKPGQSPKLLIYKVSNRFSGVPDRFSGSGSGTDFTLKISRVEAEDLGVYFCSQSTHVPYTFGGGTKLEIK

SEQ ID NO: 887 - protein, 16 aa, synthetic construct
  REGION 1..16: note = Recombinant protein
RSSQSLVHRNGNTYFH

SEQ ID NO: 888 - protein, 7 aa, synthetic construct
  REGION 1..7: note = Recombinant protein
KVSNRFS

SEQ ID NO: 889 - protein, 9 aa, synthetic construct
  REGION 1..9: note = Recombinant protein
SQSTHVPYT

SEQ ID NO: 890 - protein, 114 aa, synthetic construct
  REGION 1..114: note = Recombinant protein
EVKLLESGGGLVQPGGSLKLSCAASGFDFSRYWMTWVRQAPGKGLEWIGEINPDSSTINYNPSLKDKFIISRDNAKNTLYLQMSEVRSEDTALYYCASGVFTSWGQGTLVTVSA

SEQ ID NO: 891 - protein, 5 aa, synthetic construct
  REGION 1..5: note = Recombinant protein
RYWMT

SEQ ID NO: 892 - protein, 17 aa, synthetic construct
  REGION 1..17: note = Recombinant protein
EINPDSSTINYNPSLKD

SEQ ID NO: 893 - protein, 5 aa, synthetic construct
  REGION 1..5: note = Recombinant protein
GVFTS

SEQ ID NO: 894 - protein, 107 aa, synthetic construct
  REGION 1..107: note = Recombinant protein
DIQMTQTTSSLSASLGDRVTISCRASQDISNYLNWYQQKPDGTVKLLIFYTSRLHSGVPSRFSGSGSGTDYSLTISNLEPEDIATYYCQQDTKLPYTFGGGTKLEMK

SEQ ID NO: 895 - protein, 11 aa, synthetic construct
  REGION 1..11: note = Recombinant protein
RASQDISNYLN

SEQ ID NO: 896 - protein, 7 aa, synthetic construct
  REGION 1..7: note = Recombinant protein
YTSRLHS

SEQ ID NO: 897 - protein, 9 aa, synthetic construct
  REGION 1..9: note = Recombinant protein
QQDTKLPYT

SEQ ID NO: 898 - protein, 122 aa, synthetic construct
  REGION 1..122: note = Recombinant protein
QVQLQQSGAEPMKPGASVKISCKATGYTFSSHWIEWIKQRPGHGLEWIGEILPGSGNTNYNEKFKGKATFTADTSSNTAYMQLSSLTSEDSAVYYCARGYYGLNYDWYFDVWGAGTTVTVSS

SEQ ID NO: 899 - protein, 5 aa, synthetic construct
  REGION 1..5: note = Recombinant protein
SHWIE

SEQ ID NO: 900 - protein, 17 aa, synthetic construct
  REGION 1..17: note = Recombinant protein
EILPGSGNTNYNEKFKG

SEQ ID NO: 901 - protein, 13 aa, synthetic construct
  REGION 1..13: note = Recombinant protein
GYYGLNYDWYFDV

SEQ ID NO: 902 - protein, 112 aa, synthetic construct; identical to SEQ ID NO: 886
  REGION 1..112: note = Recombinant protein

SEQ ID NO: 903 - protein, 16 aa, synthetic construct
  REGION 1..16: note = Recombinant protein
RSSQSLVHRNGNTYFH

SEQ ID NO: 904 - protein, 7 aa, synthetic construct
  REGION 1..7: note = Recombinant protein
KVSNRFS

SEQ ID NO: 905 - protein, 9 aa, synthetic construct
  REGION 1..9: note = Recombinant protein
SQSTHVPYT

SEQ ID NO: 906 - protein, 114 aa, synthetic construct
  REGION 1..114: note = Recombinant protein
EVKLLESGGGLVQPGGSLKLSCAASGFDFSRYWMTWVRQAPGKGLEWIGEINPDSSTINYAPSLKDKFIISRDNAKNTLYLQMSEVRSEDTALYYCASGVFTSWGQGTLVTVSA

SEQ ID NO: 907 - protein, 5 aa, synthetic construct
  REGION 1..5: note = Recombinant protein
RYWMT

SEQ ID NO: 908 - protein, 17 aa, synthetic construct
  REGION 1..17: note = Recombinant protein
EINPDSSTINYAPSLKD

SEQ ID NO: 909 - protein, 5 aa, synthetic construct
  REGION 1..5: note = Recombinant protein
GVFTS

SEQ ID NO: 910 - protein, 112 aa, synthetic construct
  REGION 1..112: note = Recombinant protein
DIVMTQTPLSSPVTLGQPASISCRSSQSLVHRPGNTYFHWLQQRPGQPPRLLIYKVSNRFSGVPDRFSGSGAGTDFTLKISRVEAEDVGVYYCSQSTHVPYTFGQGTKLEIK

SEQ ID NO: 911 - protein, 16 aa, synthetic construct
  REGION 1..16: note = Recombinant protein
RSSQSLVHRPGNTYFH

SEQ ID NO: 912 - protein, 7 aa, synthetic construct
  REGION 1..7: note = Recombinant protein
KVSNRFS

SEQ ID NO: 913 - protein, 9 aa, synthetic construct
  REGION 1..9: note = Recombinant protein
SQSTHVPYT

SEQ ID NO: 914 - protein, 114 aa, synthetic construct
  REGION 1..114: note = Recombinant protein
EVQLVESGGGLVQPGGSLRLSCAASGFDFSRYWMTWVRQAPGKGLVWVGEINPDSSTINYAPSVKGRFTISRDNAKNTLYLQMNSLRAEDTAVYYCASGVFTSWGQGTLVTVSS

SEQ ID NO: 915 - protein, 5 aa, synthetic construct
  REGION 1..5: note = Recombinant protein
RYWMT

SEQ ID NO: 916 - protein, 17 aa, synthetic construct
  REGION 1..17: note = Recombinant protein
EINPDSSTINYAPSVKG

SEQ ID NO: 917 - protein, 5 aa, synthetic construct
  REGION 1..5: note = Recombinant protein
GVFTS

SEQ ID NO: 918 - protein, 112 aa, synthetic construct
  REGION 1..112: note = Recombinant protein
DIVMTQTPLSSPVTLGQPASISCRSSQSLVHRNGNTYFHWLQQRPGQPPRLLIYKVSNRFSGVPDRFSGSGAGTDFTLKISRVEAEDVGVYYCSQSTHVPYTFGQGTKLEIK

SEQ ID NO: 919 - protein, 16 aa, synthetic construct
  REGION 1..16: note = Recombinant protein
RSSQSLVHRNGNTYFH

SEQ ID NO: 920 - protein, 7 aa, synthetic construct
  REGION 1..7: note = Recombinant protein
KVSNRFS

SEQ ID NO: 921 - protein, 9 aa, synthetic construct
  REGION 1..9: note = Recombinant protein
SQSTHVPYT

SEQ ID NO: 922 - protein, 114 aa, synthetic construct
  REGION 1..114: note = Recombinant protein
EVQLVESGGGLVQPGGSLRLSCAASGFDFSRYWMTWVRQAPGKGLVWVGEINPDSSTINYAPSVKGRFTISRDNAKNTLYLQMNSLRAEDTAVYYCARGVFTSWGQGTLVTVSS

SEQ ID NO: 923 - protein, 5 aa, synthetic construct
  REGION 1..5: note = Recombinant protein
RYWMT

SEQ ID NO: 924 - protein, 17 aa, synthetic construct
  REGION 1..17: note = Recombinant protein
EINPDSSTINYAPSVKG

SEQ ID NO: 925 - protein, 5 aa, synthetic construct
  REGION 1..5: note = Recombinant protein
GVFTS

SEQ ID NO: 926 - protein, 112 aa, synthetic construct; identical to SEQ ID NO: 918
  REGION 1..112: note = Recombinant protein

SEQ ID NO: 927 - protein, 16 aa, synthetic construct
  REGION 1..16: note = Recombinant protein
RSSQSLVHRNGNTYFH

SEQ ID NO: 928 - protein, 7 aa, synthetic construct
  REGION 1..7: note = Recombinant protein
KVSNRFS

SEQ ID NO: 929 - protein, 9 aa, synthetic construct
  REGION 1..9: note = Recombinant protein
SQSTHVPYT

SEQ ID NO: 930 - protein, 114 aa, synthetic construct
  REGION 1..114: note = Recombinant protein
EVQLVESGGGLVQPGGSLRLSCAASGFTFSRYWMTWVRQAPGKGLVWVGEINPDSSTINYADSVKGRFTISRDNAKNTLYLQMNSLRAEDTAVYYCARGVFTSWGQGTLVTVSS

SEQ ID NO: 931 - protein, 5 aa, synthetic construct
  REGION 1..5: note = Recombinant protein
RYWMT

SEQ ID NO: 932 - protein, 17 aa, synthetic construct
  REGION 1..17: note = Recombinant protein
EINPDSSTINYADSVKG

SEQ ID NO: 933 - protein, 5 aa, synthetic construct
  REGION 1..5: note = Recombinant protein
GVFTS

SEQ ID NO: 934 - protein, 112 aa, synthetic construct; identical to SEQ ID NO: 838
  REGION 1..112: note = Recombinant protein

SEQ ID NO: 935 - protein, 16 aa, synthetic construct
  REGION 1..16: note = Recombinant protein
RSSQSLVHRQGNTYFH

SEQ ID NO: 936 - protein, 7 aa, synthetic construct
  REGION 1..7: note = Recombinant protein
KVSNRFS

SEQ ID NO: 937 - protein, 9 aa, synthetic construct
  REGION 1..9: note = Recombinant protein
SQSTHVPYT

SEQ ID NO: 938 - protein, 114 aa, synthetic construct; identical to SEQ ID NO: 842
  REGION 1..114: note = Recombinant protein

SEQ ID NO: 939 - protein, 5 aa, synthetic construct
  REGION 1..5: note = Recombinant protein
RYWMT

SEQ ID NO: 940 - protein, 17 aa, synthetic construct
  REGION 1..17: note = Recombinant protein
EINPDSSTINYAPSVKG

SEQ ID NO: 941 - protein, 5 aa, synthetic construct
  REGION 1..5: note = Recombinant protein
GVFTS

SEQ ID NO: 942 - protein, 326 aa, synthetic construct
  REGION 1..326: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 943 - protein, 326 aa, synthetic construct
  REGION 1..326: note = Recombinant protein
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 944 - DNA, 20 bp, synthetic construct
  misc_feature 1..20: note = reverse primer used for VH amplification
  source 1..20: mol_type = other DNA
agctgggaaggtgtgcacac

SEQ ID NO: 945 - DNA, 36 bp, synthetic construct
  misc_feature 1..36: note = reverse primer used for VL amplification
  source 1..36: mol_type = other DNA
gctctagactaacactcattcctgttgaagctcttg